(19) United States
(12) Patent Application Publication
(10) Pub. No.: US 2024/0423834 A1
(43) Pub. Date: Dec. 26, 2024

(54) METHODS AND APPARATUS FOR SUBRETINAL DELIVERY

(71) Applicant: Alcon Inc., Fribourg (CH)

(72) Inventors: Reto Grüebler, Greifensee (CH); Venjamin Stojanov, Dietlikon (CH); Pascal Philip Koller, Wallisellen (CH)

(21) Appl. No.: 18/744,875

(22) Filed: Jun. 17, 2024

Related U.S. Application Data
(60) Provisional application No. 63/509,570, filed on Jun. 22, 2023.

(51) Int. Cl. A61F 9/00; A61B 17/34; A61B 90/50; A61M 25/00; A61M 25/01; A61M 25/04; A61M 25/06

(52) U.S. Cl. CPC A61F 9/0017; A61B 2017/3409; A61B 90/50; A61M 25/0032; A61M 2025/0047; A61M 25/007; A61M 2025/0092; A61M 25/0097; A61M 25/0138; A61M 25/04; A61M 25/0662

(57) ABSTRACT

The present disclosure generally relates to devices for ophthalmic procedures, and more particularly, to apparatus for performing subretinal injections and methods of use thereof. In some embodiments, the apparatus includes an injection needle having a proximal end and a distal end, the distal end configured to be insertable into the subretinal space at a position on a surface of a retina. The apparatus may further include an inserter device removably coupled to the injection needle and a tubing having a distal end coupled to the proximal end of the injection needle and a proximal end coupled to a fluid source. The tubing may have a first lumen and a second lumen, wherein the tubing is disposed through the inserter device. The apparatus may further include a stabilizer configured to immobilize the injection needle at the position on the surface of the retina and a fluid source. The fluid source may include both a first fluid reservoir containing a non-treatment solution and a second fluid reservoir containing a treatment solution. The fluid source may be configured to provide the non-treatment solution from the first fluid reservoir to the subretinal space via the first lumen. The fluid source may be further configured to provide the treatment solution from the second fluid reservoir into the subretinal space via the second lumen.

### BACKGROUND

The human eye comprises three main layers: a protective outer layer of opaque, white membrane known as the sclera; a thin middle layer known as the choroid; and an innermost, light-sensitive layer, known as the retina, which lines the back two-thirds of the eye. The retina consists of two sublayers: the sensory (or neural) retina, which includes photoreceptor cells (e.g., rods and cones), that convert light images into electrochemical signals; and the retinal pigment epithelium (RPE). Cells of the RPE absorb scattered light and transport oxygen, nutrients, and cellular waste between the sensory retina and the choroid to maintain homeostasis therebetween. The RPE is separated from the inner sensory retina by the subretinal space.

Certain diseases of the eye are treatable via injection into the subretinal space, including, e.g., age-related macular degeneration (AMD) and retinal degenerative diseases and genetic defects. Typical practice requires at least two persons to administer the subretinal injection. For example, a lead surgeon may guide the injection instrument, e.g., a syringe/needle, and visually monitor the injection site, while a skilled surgical assistant pushes the fluid from the syringe and monitors the injection volume. Accordingly, typically, a first syringe is prepared with a small gauge needle and containing a non-treatment fluid, e.g., balanced salt solution (BSS). In the first step of the procedure, the first syringe is inserted through the retina into the subretinal space. While the surgeon handles the first syringe and visually monitors the injection site, the assistant manually injects the non-treatment fluid and monitors the injection volume. Next, the first syringe is removed from the eye.

A second syringe is prepared with a small gauge needle and containing a treatment fluid, e.g., including a therapeutic. In the second step of the procedure, the second syringe is inserted through the retina into the subretinal space at about the same location as the first syringe. While the surgeon handles the second syringe and visually monitors the injection site, the assistant manually injects the treatment fluid and monitors the injection volume. Consequently, there are many disadvantages with using a handheld injection instrument to manually control the injection in a two-step process. Some of these disadvantages are described below.

First, performing the injection with the injection instrument being handheld, as described above, can result in tearing of the retina. In particular, tearing of the retina can result from inadvertent movement of the syringe/needle due to external forces from outside the eye while the needle is inserted through the retina. The external forces may include inadvertent movements on the part of the surgeon during handling of the syringe or on the part of the assistant during manual control of the fluid injection.

Furthermore, manual control of the fluid injection, as described above, can have a number of additional disadvantages. Typically, manual control of the fluid injection involves manual depression of the plunger. For example, manual control of the fluid injection can result in incorrect injection volume, which can result in over- or under-dosing or excessive retinal stretch. In another example, manual control of the fluid injection can result in a high flow velocity into the subretinal space, which can damage the retina or the RPE, e.g., causing rhegmatogenous-like retinal detachment with changes in retinal morphology or RPE atrophy. In yet another example, manual control of the fluid injection can result in a high shear force in the needle, which can be detrimental to the biologic activity of various therapeutics, e.g., drugs, stem cells, viral vectors, carried by the injection fluid.

In addition, removing the first needle and inserting the second needle through the retina, as described above, can have further disadvantages. For example, making several insertions through the retina can contribute to retinal tearing. In another example, forming two different holes in the retina, one for each injection step, increases the invasiveness of the procedure (e.g., damage to the retina) and the potential for fluid to leak from the subretinal space. And, in some examples of current manual injection methods, the injected composition may remain localized in the subretinal space near the injection site, and may not reach a desired tissue (e.g., the macula).

Each of the problems described above can negatively affect the ophthalmic treatment being administered and/or carry an increased safety risk. Therefore, what is needed in the art are improved devices for ophthalmic treatment including an improved apparatus and method for subretinal delivery.

### SUMMARY

Embodiments of the present disclosure generally relate to devices for ophthalmic procedures, and more particularly, to apparatus and methods for performing subretinal injection.

Certain embodiments of the present disclosure provide an apparatus for performing a subretinal injection into a subretinal space of an eye, the apparatus comprising: an injection needle having a proximal end and a distal end, the distal end configured to be insertable into the subretinal space at a position on a surface of the retina; an inserter device removably coupled to the injection needle; a tubing having a distal end coupled to the proximal end of the injection needle and a proximal end coupled to a fluid source, the tubing having a first lumen and a second lumen, wherein the tubing is disposed through the inserter device; a stabilizer configured to immobilize the injection needle at the position on the surface of the retina; and the fluid source having a first fluid reservoir containing a non-treatment solution and a second fluid reservoir containing a treatment solution, wherein the fluid source is configured to provide the non-treatment solution from the first fluid reservoir to the subretinal space via the first lumen, and wherein the fluid source is configured to provide the treatment solution from the second fluid reservoir into the subretinal space via the second lumen.

Certain embodiments of the present disclosure provide a method of performing a subretinal injection into a subretinal space of an eye, the method comprising: inserting a distal end of an injection needle into the subretinal space at a target site on a surface of a retina, the injection needle having a proximal end coupled to a distal end of a tubing, the tubing having a proximal end coupled to a fluid source, the proximal end of the injection needle further removably coupled to a distal end of an inserter device; immobilizing the injection needle at the target site on the surface of the retina by applying a pressure or fluid through a first lumen of the tubing to extend a stabilizer beyond a distal end of the first lumen to contact the surface of the retina; decoupling the inserter device from the injection needle; providing a non-treatment solution from the fluid source to the subretinal space via a second lumen of the tubing; and providing a treatment solution to the subretinal space via a third lumen of the tubing using the fluid source.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIGS. 1A and 1B illustrate cross-sectional views of an eye, according to certain embodiments described herein.

FIG. 2 illustrates a cross-sectional view of an eye during performance of a subretinal injection procedure via a transvitreal approach, according to certain embodiments of the present disclosure.

FIG. 3 illustrates a cross-sectional view of an eye during performance of a subretinal injection procedure via a suprachoroidal approach, according to certain embodiments of the present disclosure.

FIG. 4 illustrates a perspective view of an exemplary surgical system for performing subretinal injection procedures, according to certain embodiments of the present disclosure.

FIG. 5 illustrates a perspective view of an exemplary subretinal delivery device, according to certain embodiments of the present disclosure.

FIG. 6 illustrates a perspective view of an exemplary subretinal delivery device, according to certain embodiments of the present disclosure.

FIGS. 7A and 7B illustrate schematic, cross-sectional side views of a distal end of an exemplary injection cannula, according to certain embodiments of the present disclosure.

FIGS. 8A-8C illustrate schematic, cross-sectional side views of a distal end of an exemplary injection cannula, according to certain embodiments of the present disclosure.

FIG. 9A illustrates a perspective view of an exemplary injection needle, according to certain embodiments of the present disclosure. FIGS. 9B-9D illustrate schematic cross-sectional side views of the exemplary injection needle of FIG. 9A, according to certain embodiments of the present disclosure.

FIG. 10A illustrates a schematic cross-sectional side view of the exemplary injection needle, according to certain embodiments of the present disclosure. FIG. 10B illustrates a schematic side view of the exemplary injection needle of FIG. 10 during use, according to certain embodiments of the present disclosure.

FIGS. 11A-11B illustrate perspective views of an exemplary injection needle, according to certain embodiments of the present disclosure.

FIG. 12 illustrates a schematic, cross-sectional side view of a distal end of an exemplary injection cannula, according to certain embodiments of the present disclosure.

FIGS. 13A-13B illustrate schematic cross-sectional side views of an exemplary subretinal delivery device, according to certain embodiments of the present disclosure.

FIGS. 14A-14B illustrate perspective side views of an exemplary subretinal delivery device and injection cannula, according to certain embodiments of the present disclosure.

FIG. 15A illustrates a schematic view of an exemplary subretinal delivery system, according to certain embodiments of the present disclosure.

FIG. 15B illustrates an enlarged cross-sectional view of a multi-lumen tubing in FIG. 15A, according to certain embodiments of the present disclosure.

FIG. 15C illustrates a top isometric view of a portion of the delivery system of FIG. 15A, according to certain embodiments of the present disclosure.

FIG. 15D illustrates a schematic view of the delivery system of FIG. 15A in conjunction with an exemplary subretinal delivery device, according to certain embodiments of the present disclosure.

FIG. 15E illustrates an enlarged side sectional view of a portion of the delivery system shown in FIG. 15D, according to certain embodiments of the present disclosure.

FIG. 15F illustrates a top isometric view of a portion of the delivery system with an alternative injection needle and stabilizer, according to certain embodiments of the present disclosure.

FIGS. 16A-16E illustrate transverse sectional views of an eye at different steps of performing a subretinal injection with the delivery system of FIGS. 15A-15E, according to certain embodiments of the present disclosure.

FIGS. 17A-17C illustrate cross-sectional side views of an exemplary subretinal delivery device, according to certain embodiments of the present disclosure.

FIG. 18A illustrates a perspective view of an exemplary subretinal delivery device, according to certain embodiments of the present disclosure.

FIG. 18B illustrates a perspective view of another exemplary subretinal delivery device, according to certain embodiments of the present disclosure.

FIG. 19A illustrates a perspective view of an exemplary injection cannula, according to certain embodiments of the present disclosure.

FIG. 19B illustrates a perspective view of another exemplary injection cannula, according to certain embodiments of the present disclosure.

FIG. 19C illustrates cross-sectional views of exemplary injection cannula profiles, according to certain embodiments of the present disclosure.

FIG. 20A illustrates a cross-sectional top view of an exemplary injection cannula, according to certain embodiments of the present disclosure.

FIGS. 20B and 20C illustrate cross-sectional side views of alternative arrangements of the exemplary injection cannula of FIG. 20A, according to certain embodiments of the present disclosure.

FIGS. 20D and 20E illustrate transverse sectional views of an eye at different steps of performing a subretinal injection with the exemplary injection cannula of FIG. 20A, according to certain embodiments of the present disclosure.

FIGS. 21A and 21B illustrate perspective views of an exemplary distal tip of an injection cannula, according to certain embodiments of the present disclosure.

FIG. 21C illustrates a cross-sectional side view of the exemplary injection cannula distal tip of FIGS. 21A and 21B, according to certain embodiments of the present disclosure.

FIG. 22A illustrates a perspective view of an exemplary distal tip of an injection cannula, according to certain embodiments of the present disclosure.

FIG. 22B illustrates a cross-sectional side view of the exemplary injection cannula distal tip of FIG. 22A, according to certain embodiments of the present disclosure.

FIGS. 23A and 23B illustrate cross-sectional side views of an exemplary internal ramp assembly for a distal tip of an injection cannula, according to certain embodiments of the present disclosure.

FIGS. 24A and 24B illustrate schematic perspective views of an exemplary distal tip of an injection cannula, according to certain embodiments of the present disclosure.

FIG. 25 illustrates a schematic perspective view of an exemplary distal tip of an injection cannula, according to certain embodiments of the present disclosure.

FIGS. 26A and 26B illustrate perspective views of exemplary subretinal delivery device, according to certain embodiments of the present disclosure.

FIGS. 27A and 27B illustrate various perspective views of an exemplary subretinal delivery device, according to certain embodiments of the present disclosure.

FIG. 28A illustrates a cross-sectional side view of an exemplary guidance cannula, according to certain embodiments of the present disclosure.

FIG. 28B illustrates a cross-sectional top view of the exemplary guidance cannula of FIG. 28A, according to certain embodiments of the present disclosure.

FIGS. 28C and 28D illustrate transverse sectional views of an eye at different steps of performing a subretinal injection with the exemplary guidance cannula of FIG. 28A, according to certain embodiments of the present disclosure.

FIG. 29A illustrates a perspective view of an exemplary entry cannula, according to certain embodiments of the present disclosure.

FIGS. 29B and 29C illustrate transverse sectional views of an eye at different steps of performing a subretinal injection with the exemplary entry cannula of FIG. 29A, according to certain embodiments of the present disclosure.

FIGS. 30A and 30B illustrate perspective views of an exemplary entry cannula, according to certain embodiments of the present disclosure.

FIGS. 31A-31C illustrate schematic cross-sectional views of exemplary subretinal delivery devices, according to certain embodiments of the present disclosure.

FIGS. 32A-32D illustrate side schematic views of exemplary support arms for supporting a delivery device during a subretinal injection procedure, according to certain embodiments described herein.

FIG. 33A illustrates an example operating environment during the performance of a subretinal injection procedure, according to certain embodiments of the present disclosure.

FIG. 33B illustrates various components of the operating environment in FIG. 33A, according to certain embodiments of the present disclosure.

FIGS. 34A-34D illustrate transverse sectional views of a portion of an eye at different steps of performing an exemplary subretinal injection procedure with post-injection sealing, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as used herein, a distal end of a component refers to the end that is closer to a patient's body while the proximal end of the component refers to the end that is facing away from the patient's body.

As used herein, the term “surgical system” may refer to any surgical system, console, or device for performing a surgical procedure. For example, the term “surgical system” may refer to a surgical console, such as a phacoemulsification console, a vitrectomy console, a laser system, or any other consoles, systems, or devices used in an ophthalmic operating room, as known to one of ordinary skill in the art. Note that although certain embodiments herein are described in relation to ophthalmic systems, tools, and environments, the embodiments described herein are similarly applicable to other types of medical or surgical systems, tools, and environments.

As used herein, the term “about” may refer to a +/−10% variation from the nominal value. It is to be understood that such a variation can be included in any value provided herein.

Although generally described with reference to ophthalmic surgical devices and systems, the devices and systems described herein may be implemented with other devices and systems, such as devices and systems for other surgeries, without departing from the scope of the present application.

Embodiments of the present disclosure generally relate to devices and methods for ophthalmic treatment, and more particularly, to apparatus and methods for performing subretinal injection. Subretinal injection generally refers to delivery of fluid or other therapeutic substances or stem cells into a subretinal space between a retina and a retinal pigment epithelium (RPE) of an eye.

It has been suggested that cell-based therapies, wherein cells such as stem cells are engrafted into/adjacent a target treatment site, may prove efficacious for several currently untreatable conditions involving the RPE layer, including AMD and retinitis pigmentosa (AR). Cell transplantation into the human retina has the potential to restore lost vision and to provide treatment of advanced stages of retinal degeneration with significant RPE loss. Similarly, gene therapies, wherein foreign DNA (Deoxyribonucleic acid) constructs are introduced into host cells to modify their activity, also hold much potential in treating retinal diseases such as AMD, AR, choroidermia, and the like. However, in order to treat retinal conditions, such techniques require entry into the subretinal space. And, as described above, current techniques for injection into the subretinal space suffer from many disadvantages, as the tissues surrounding the subretinal space are delicate and require a high level of skill to maneuver around.

Thus, embodiments of the present disclosure provide improved methods and apparatus for performing subretinal injection that mitigate or even eliminate the drawbacks associated with current techniques.

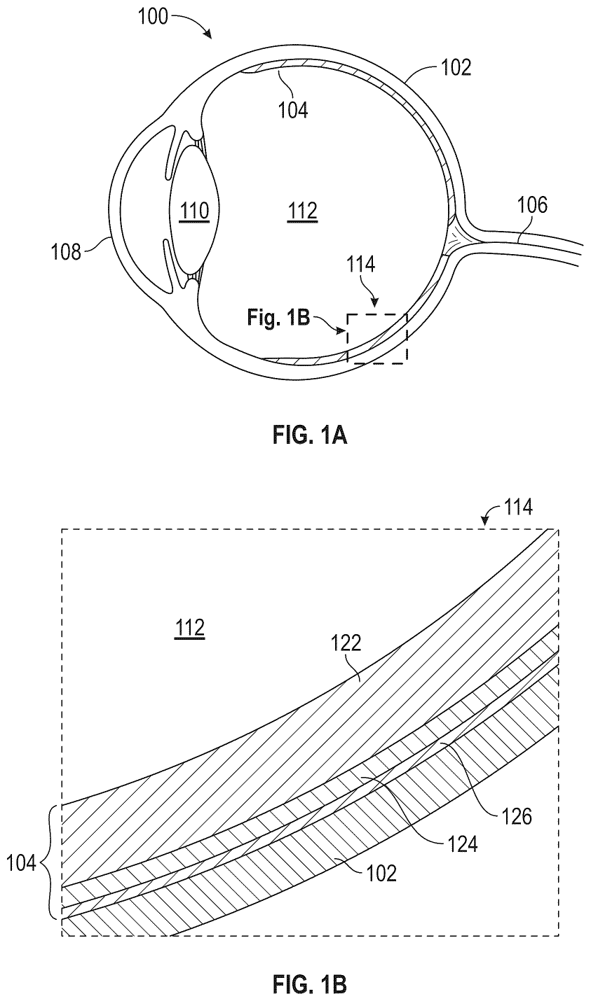

FIG. 1A illustrates a cross-sectional view of an eye 100. A number of features of the eye 100 are illustrated herein. The eye 100 includes a sclera 102 that is coupled to a retinal membrane or retina 104 by a choroid (not illustrated in FIG. 1A). The choroid includes connective tissue to attach the retina 104 to the inside wall of the sclera 102 at the back of the eye 100 and to provide oxygen and nourishment to the outer layers of the retina 104. A cornea 108 permits light to enter the eye 100, the light being focused by a lens 110 through a vitreous chamber 112 onto the retina 104, which contains photo-activated cells that transmit signals over the optic nerve 106 to the brain.

Problems may develop in the eye that prevent the proper development and/or function of the retina as it provides signals to the brain for processing into cognizable images. A potential treatment or therapy for such eye problems may include delivering genetic material and/or stem cells into a desired region of the subretinal space, the area between the outermost surface of the retina and the retinal pigment epithelium (RPE), just above the choroid, where the immune response may be sufficiently subdued.

An area of interest 114 is shown in FIG. 1A on a lower portion of the eye 100. The area of interest 114 is shown in more detail in FIG. 1B.

Referring now to FIG. 1B, the area of interest 114 of the eye 100 is shown in close-up to provide greater detail of the layers of the retina 104. Note the layers are not drawn to scale. As shown in FIG. 1A, the retina 104 includes several layers, including a main retinal layer 122, a subretinal space 124, and an opaque layer 126. The retinal layer 122 includes an inner limiting membrane that is in contact with the vitreous humor that fills the vitreous chamber 112. The retinal layer 122 further includes a nerve fiber sub-layer, a ganglion cell sub-layer, an inner plexiform sub-layer, an inner nuclear sub-layer, an outer plexiform sub-layer, and an outer nuclear sub-layer. The retinal layer 122 also includes an external limiting membrane and a photoreceptor sub-layer. The opaque layer 126 includes the retinal pigment epithelium (RPE) and the choroid.

When therapeutic agents are delivered to the retina 104, the fluid containing the therapeutic agents is delivered between the retinal layer 122 and the retinal pigment epithelium of the opaque layer 126, i.e., in the subretinal space 124. Conventionally, fine needle is used to puncture the retinal layer 122 to allow the fluid containing the therapeutic agents into this subretinal space. In some examples, a bleb may then be formed by the injection of, e.g., a balanced salt solution (BSS), and then a fluid containing therapeutic agents is injected into the space formed by the bleb. The formation of the bleb may provide the space in which to inject the therapeutic agents without subjecting them to the fluid pressures necessary to form that space. In some examples, a single injection may be used to form the bleb and introduce the therapeutic agents. The fluid containing the therapeutic agents is introduced into the subretinal space 124 between the photoreceptor sub-layer and the retinal pigment epithelium, where immune system reactions to the therapeutic agents may be relatively subdued.

During the subretinal delivery process, care must be taken to avoid: retinal tears, as caused by creating a bleb with high retinal tension or unwanted movement of an injection needle; puncturing of the retinal pigment epithelium of the opaque layer 126 by the injection needle; damage to the retinal pigment epithelium, as caused by excessive injection flow rates, resulting in rhegmatogenous-like retinal detachment with changes in retinal morphology; and back flow or reflux (e.g., spilling) of therapeutic agents through the puncture in retinal layer 122 into the vitreous chamber 112, all of which are problems associated with current subretinal injection devices and methods. Accordingly, the systems, apparatus, and methods of the present disclosure, embodiments of which are described herein, enable performance of subretinal injections while avoiding the above situations by facilitating: efficient access to the subretinal space and proper positioning of a delivery device needle tip in the retina 104; stabilization of the delivery device needle and/or delivery device handpiece to reduce effects of unwanted movements by a user; and improved fluidic control/handling of fluids being injected with reduced spilling into the vitreous chamber 112.

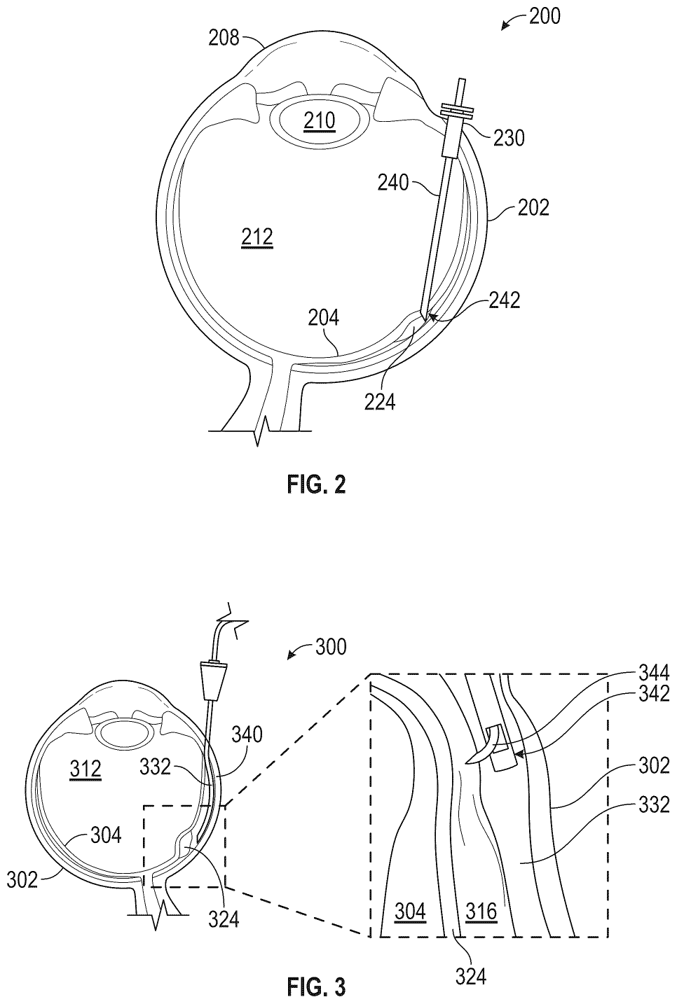

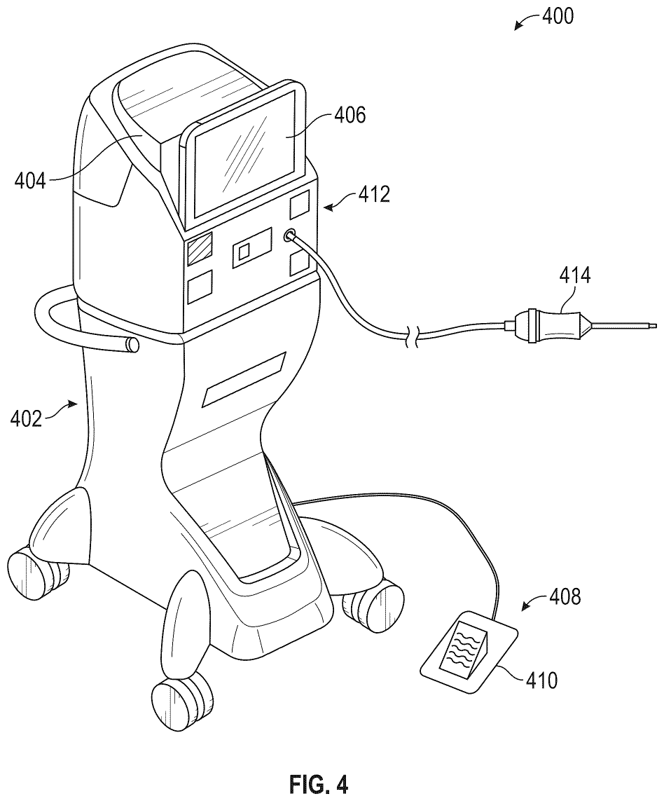

Generally, there are two main approaches for administering subretinal injections: (1) a transvitreal approach, which is illustrated in FIG. 2; and (2), a suprachoroidal approach, which is illustrated in FIG. 3. Embodiments of the present disclosure may be utilized with one or both of these approaches, as is discussed in greater detail below.

As shown in the cross-sectional view of eye 200 in FIG. 2, in a transvitreal approach, an injection cannula 240 of a delivery device may be inserted through a valved insertion cannula 230 (or other entry cannula) disposed through an incision in the sclera 202 (i.e., a sclerotomy) of the eye 200 and guided through the vitreous chamber 212 toward the retina 204. In certain embodiments, the sclera 202 may be incised utilizing a trocar cannula, which may consist of the valved insertion cannula 230 and a trocar. Typically, the trocar cannula, having a hub at a proximal end thereof, is inserted into the eye 200 (thereby forming the incision) until a bottom surface of the hub contacts the sclera 202. Then, the trocar is removed from the eye 200, leaving the valved insertion cannula 230 in place as shown in FIG. 2. In certain embodiments, rather than being inserted through the sclera (e.g., transscleral), the valved insertion cannula 230 and thus, the injection cannula 240 of the delivery device may be inserted into the eye 200 through the cornea 208, wherein the injection cannula 240 is bypassed around the lens 210 and into the vitreous chamber 212 (e.g., transcorneal).

The injection cannula 240 of the delivery device is guided through the vitreous chamber 212 until a distal end 242 thereof is positioned adjacent to the retina 204 and near a target injection site in the subretinal space 224. At this point, an injection needle of the delivery device, which may be disposed within the injection cannula 240 and is configured to slidably extend from the distal end 242, may be inserted through the retina 204 and into the subretinal space 224, e.g., between the outermost neural layer of the retina 204 and the retinal pigment epithelium, for injection.

As shown in the cross-sectional view of eye 300 in FIG. 3, in a suprachoroidal approach, a flexible injection cannula 340 of a delivery device may be inserted through an incision in the sclera 302 of the eye 300 and guided through the suprachoroidal space (SCS) 332 to the target injection site, without being passed through the vitreous chamber 312. In certain embodiments, a valved cannula or other entry cannula, similar to entry cannula 230, may be utilized to facilitate entry of the injection cannula 340 into the eye 300 through the sclera 302. The suprachoroidal space 332 is a potential space between the sclera 302 and choroid 316 of the eye 300 that traverses the circumference of the posterior segment of the eye 300. Once a distal end 342 of the flexible injection cannula 340 in the suprachoroidal space 332 is positioned adjacent to the target injection site in the subretinal space 324, an injection needle 344 of the delivery device, which may be disposed within the injection cannula 340 and configured to slidably extend from the distal end 342, is inserted through the choroid 316 and into the subretinal space 324 for injection, as compared to being inserted through retina 304 in a transvitreal approach. In certain embodiments, the injection cannula 340 comprises a microcannula and the injection needle 344 comprises a microneedle.

FIG. 4 illustrates a perspective view of an exemplary surgical system 400 that may be utilized with embodiments of the present disclosure to perform subretinal injection procedures. In certain examples, surgical system 400 comprises a surgical system for ophthalmic procedures, such as retinal procedures and treatments, and may include but is not limited to surgical systems sold by Alcon of Fort Worth, Texas. The surgical system 400 includes a console 402, controller 404 (e.g., a computer unit) having a processor and memory, and an associated display 406. The display 406 may display, for example, data relating to system operation and/or system performance during a surgical procedure, which may be arranged in a graphical user interface (GUI).

Generally, the console 402 includes one or more systems or subsystems that enable a surgeon to perform a variety of surgical procedures, such as retinal procedures. For example, the subsystems that may be used together to perform a vitrectomy surgical procedure prior to the injection of therapeutic agents in order to provide improved access to the retina. In certain embodiments, the subsystems include a control system that has one or more of a foot pedal subsystem 408 including a foot pedal 410 having a number of foot-actuated controls, and a device control system or subsystem 412 in communication with a hand-held surgical instrument, shown as delivery device 414. Another subsystem may be used to provide tracking of a distal end of the delivery device 414. This may be done using optical coherence tomography (OCT), by using a displacement sensor, or by other appropriate mechanisms. The tracking information and other information may be provided to the display 406 or to a surgical microscope heads-up display. Some embodiments of the console 402 may further include a vitrectomy cutter subsystem with a vitrectomy hand piece and pump/vacuum that can also be controlled using the foot pedal 410 and/or the device control subsystem 412. These subsystems of console 402 may overlap and cooperate to perform various aspects of a procedure and may be operable separately and/or independently from each other during one or more procedures. That is, some procedures may utilize one or more subsystems while not using others.

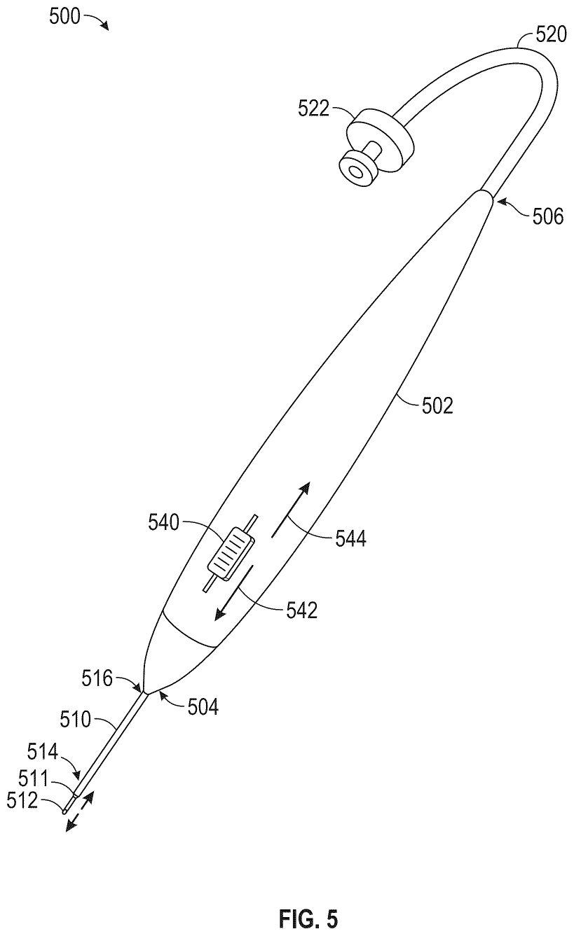

Referring now to FIG. 5, an exemplary subretinal delivery device 500 is illustrated in perspective view, according to certain embodiments of the present disclosure. The delivery device 500 may be used as the delivery device 414 of the surgical system 400 of FIG. 4, and aspects thereof may be combined with other delivery devices and/or components described herein without limitation.

The delivery device 500 includes a handle 502 and an injection cannula 510 having a proximal end 516 coupled to and extending distally from a distal end 504 of the handle 502. The injection cannula 510, which may comprise a tube, is generally formed of any suitable surgical-grade materials, such as metallic or thermoplastic polymeric materials. Examples of metallic materials include aluminum, stainless steel, and other metallic alloys. Examples of suitable thermoplastic polymeric materials include polyether ether ketone (PEEK), polyetherketone (PEK), and polytetrafluoroethylene (PTFE).

As further shown in FIG. 5, a curved or substantial straight injection needle 512 is disposed within the injection cannula 510 for piercing a desired ocular tissue (e.g., the retina or choroid) to deliver a fluid to the subretinal space. In exemplary embodiments, the injection cannula 510 is a 23-, 25-, or 27-gauge needle, while the injection needle 512 is a finer gauge needle, such as a 38-gauge needle. However, other sizes/gauges of injection cannulas and injection needles may be used in other embodiments.

In certain embodiments, the injection needle 512 is configured to slidably extend from and retract into a distal tip 511 at a distal end 514 of the injection cannula 510, which facilitates the prevention of damage to the injection needle 512 during insertion and/or movement of the injection cannula 510 in an eye. Such actuation of the injection needle 512 may be controlled by any suitable mechanism. In the example of FIG. 5, actuation of the injection needle 512 is controlled by a toggle 540 of the handle 502, which may be directly or indirectly coupled to the injection needle 512. In certain embodiments, the toggle 540 comprises a sliding button or switch, wherein sliding of the toggle 540 by a user (e.g., a surgeon) in a distal direction 542 causes the injection needle 512 to extend from the injection cannula 510, and sliding of the toggle 540 in a proximal direction 544 causes the injection needle 512 to retract into the injection cannula 510.

In certain embodiments, a sliding toggle 540 may also be lockable, such that the injection needle 512 may be fixed in either an extended or a retracted position. Locking of the injection needle 512 prevents unintended movement of the injection needle 512 during a retinal procedure, e.g., a subretinal injection, thereby reducing the risk of unwanted tissue damage and improving the overall safety of such procedures. In one example, to unlock/release the sliding toggle 540 for adjustment, the toggle 540 may be continuously depressed by a user, allowing the user to freely slide the toggle 540 and thus, freely extend or retract the injection needle 512. In this example, the toggle 540 may only be movable while depressed (e.g., activated) by the user. Correspondingly, releasing the toggle 540 may cause the toggle 540 to raise and lock in place, thereby locking the injection needle 512 in place. Such a push button locking mechanism may be facilitated, in part, by a spring lever disposed with the handle 502, as well as one or more tracks comprising grooves or notches along which the toggle 540 may slide.

As further shown in FIG. 5, in certain embodiments, a flexible fluidic tubing 520 for supplying an injection fluid, e.g., a non-treatment and/or a treatment solution, to the delivery device 500 may be disposed through a proximal end 506 of the handle 502 and fluidically coupled to the injection needle 512 within the handle 502. In certain embodiments, the fluidic tubing 520 may couple to the proximal end 506 of the handle 502, or another fluidic tubing within the handle 502 (described elsewhere herein). Generally, the fluidic tubing 520 comprises a supply line through which injection fluids (e.g., a non-treatment and/or a treatment solution) from a fluid source (not shown in FIG. 5) may be provided to the delivery device 500 for delivery to an eye. In certain embodiments, the fluidic tubing 520 comprises a multi-lumen tubing, which provides a plurality of parallel flow paths from separate fluid reservoirs of the fluid source to the injection needle 512 so that injection of multiple fluid types can be performed using only one needle. In certain embodiments, the fluid source comprises a fluidic system, which may be coupled to the fluidic tubing 520 via connection 522, such as a Luer lock or other male-female coupling. In certain other embodiments, the handle 502 may comprise an actuatable internal chamber fluidically coupled to the injection cannula 510 and containing the injection fluids. In such embodiments, subretinal delivery device 500 may not be coupled to any external fluidic tubing.

In further embodiments, to simplify fluidic preparation for subretinal injection, an injection fluid may be provided to the delivery device 500 from a prefilled cartridge that can be coupled to a fluidic drive system of the delivery device 500, or to an external fluidic system connected to the delivery device 500 via the fluidic tubing 520. In certain embodiments, the prefilled cartridge comprises a single lumen containing a premixed therapeutic substance. In other embodiments, the prefilled cartridge comprises two or more lumens containing unmixed therapeutic substances, which can be automatically or semi-automatically mixed within, e.g., a fluidic system or the delivery device, before performing subretinal injection. Cartridges for therapeutic agents are described in further detail below.

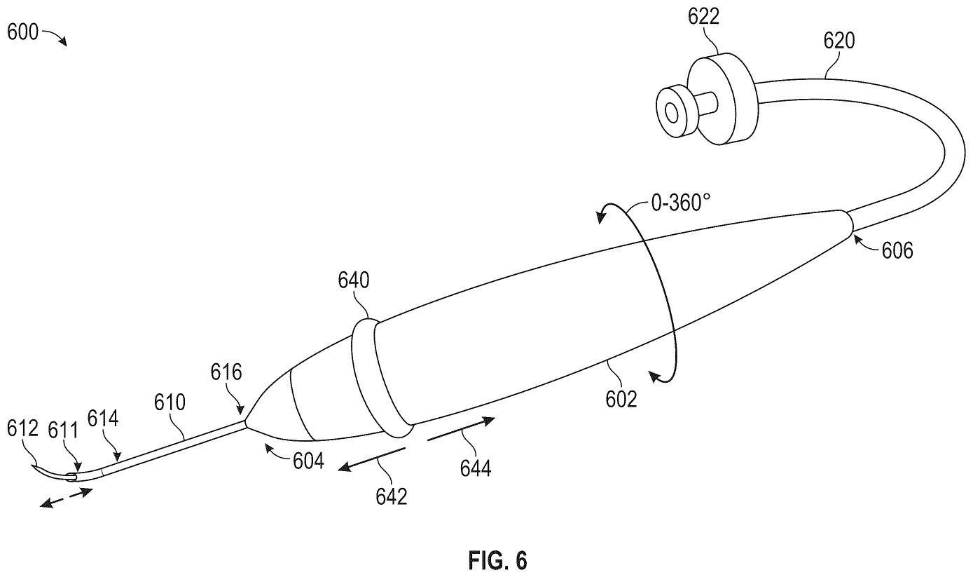

Referring now to FIG. 6, another exemplary subretinal delivery device 600 is illustrated in perspective view according to certain embodiments of the present disclosure. The delivery device 600 is substantially similar to delivery device 500, and may also be used as the delivery device 414 of the surgical system 400 of FIG. 4. Aspects of the delivery device 600 may also be combined with other delivery devices and/or components described herein without limitation. Unlike delivery device 500, however, the handle of delivery device 600 is “rotatable,” as described below.

As shown in FIG. 6, the delivery device 600 includes a handle 602, a tubular injection cannula 610 having a proximal end 616 coupled to and extending from a distal end 604 of the handle 602, and a curved or substantial straight injection needle 612 disposed within the injection cannula 610 (a curved injection needle 612 is shown). In certain embodiments, a flexible fluidic tubing 620 for supplying injection fluids (e.g., a non-treatment and/or a treatment solutions) to the delivery device 600 may be disposed through a proximal end 606 of the handle 602 and fluidically coupled to the injection needle 612 within the handle 602. Alternatively, the fluidic tubing 620 may couple to the proximal end 606 of the handle 602, or another fluidic tubing within the handle 602. In certain embodiments, the fluidic tubing 620 comprises a multi-lumen tubing. Like fluidic tubing 520, the fluidic tubing 620 comprises a connection 622 disposed at a proximal end of the fluidic tubing 620 to couple to a fluid source, such as a fluidic system integrated with a surgical console.

The injection needle 612 is configured to slidably extend from and retract into a distal tip 611 at a distal end 614 of the injection cannula 610 to prevent damage to the injection needle 612. In FIG. 6, actuation of the injection needle 612 is controlled by a circumscribing toggle 640, which may completely circumscribe or wrap around the handle 602 (e.g., around longitudinal major axis X of the handle 602) near the distal end 604 and may directly or indirectly couple to the injection needle 612 within the handle 602. Similar to the toggle 540 above, sliding the toggle 640 in a distal direction 642 causes the injection needle 612 to extend from the injection cannula 610, while sliding the toggle 640 in a proximal direction 644 causes the injection needle 612 to retract into the injection cannula 610. Because the circumscribing toggle 640 wraps around the entirety of handle 602, a user (e.g., a surgeon) may control the extension and retraction of the injection needle 612 while the delivery device 600 is disposed at any given rotation angle in the user's hand. Accordingly, the handle 602 of the delivery device 600 may be described as “rotatable.” This rotatability is particularly beneficial with a curved injection needle 612. For example, when using such a curved injection needle 612, it may be beneficial for a user to rotate the handle 602 and thus, the injection needle 612 coupled thereto, to one side or another for accurate fluid delivery to a target injection site. The circumscribing toggle 640 therefore facilitates control of the extension/retraction of the injection needle 612 independently of handle rotation in these instances.

In certain embodiments, rather than a continuous toggle around the handle 602, the circumscribing toggle 640 comprises a plurality of buttons (e.g., 3, 4, or more buttons) symmetrically or asymmetrically distributed around the handle 602. In certain embodiments, the circumscribing toggle 640 is lockable, such that the injection needle 612 may be fixed in either an extended or retracted position.

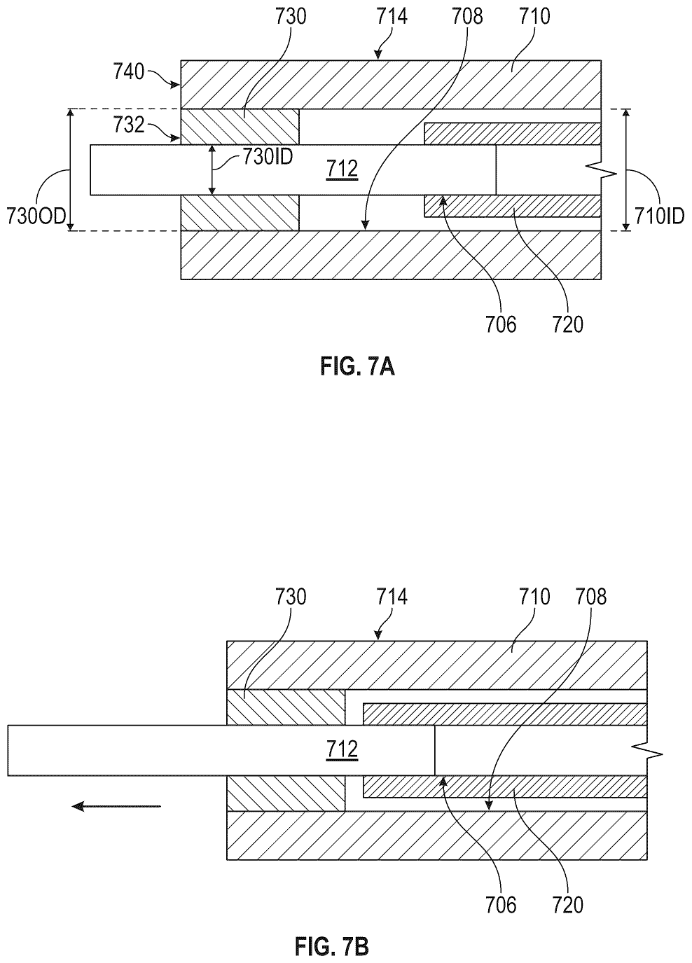

FIGS. 7A and 7B illustrate schematic, cross-sectional side views of a distal end 714 of an exemplary injection cannula 710, according to certain embodiments of the present disclosure. The injection cannula 710 is an exemplary tubular injection cannula that may be utilized with the delivery devices 500 and 600 of FIGS. 5 and 6, or other delivery devices for subretinal injection as described herein. Aspects of the injection cannula 710 may be combined with other delivery devices and/or components described herein without limitation.

As shown, an injection needle 712 is disposed within the injection cannula 710 and is configured to slidably extend from and retract into the distal end 714 thereof. Within the injection cannula 710, a proximal end 706 of the injection needle 712 is coupled to an inner fluidic shaft (or connector) 720, which may provide a fluidic coupling between injection needle 712 and a flexible fluidic tubing for supplying injection fluids to the injection needle 712. In such embodiments, the inner fluidic shaft 720 is configured to slidably translate within the injection cannula 710 to facilitate extension and retraction of the injection needle 712. Alternatively, the injection needle 712 may be coupled directly to the fluidic tubing. In certain embodiments, such fluidic tubing comprises a multi-lumen tubing, which provides a plurality of parallel flow paths from separate fluid reservoirs of a fluid source to the injection needle 712 so that an injection can be performed using only one needle.

An annular insert 730 is also disposed within the injection cannula 710 at the distal end 714 thereof and around the injection needle 712. The annular insert 730 circumscribes the injection needle 712 and functions as a mechanical stiffening or stabilizing agent for the injection needle 712 by preventing or reducing lateral movement thereof during use. In certain embodiments, stiffness of the injection needle 712 may be adjusted, or controlled, by extending or retracting the injection needle 712 from/to the injection cannula 710 and through the annular insert 730. For example, to increase the flexibility of the injection needle 712 and reduce stiffness, the injection needle 712 may be extended through the annular insert 730 and out of the injection cannula 710, as shown in FIG. 7A. Exposing a greater portion of the injection needle 712 from the injection cannula 710 (e.g., forming a longer, “freely-hanging” needle) allows the needle to be more flexible. Conversely, to increase the stiffness of the injection needle 712 for puncturing a tissue, the injection needle 712 may be retracted through the annular insert 730 and toward/into the injection cannula, as shown in FIG. 7B. Reducing a length of the injection needle 712 exposed from the injection cannula 710 (e.g., forming a shorter, “freely-hanging” needle) reduces the flexibility of the needle.

Accordingly, the stiffness adjustability of the injection needle 712 enables a user, e.g., a surgeon, to adjust to a high stiffness for easy puncturing by the injection needle 712 during subretinal injections. A higher stiffness reduces the required amount of pressure needed to puncture the retina (or other ocular tissues/membranes), and further reduces the risk or occurrence of tissue damage as caused by traction of the injection needle 712 on such tissues during puncturing. At the same time, the stiffness adjustability of the injection needle 712 enables a user to adjust to a low stiffness (or high flexibility) after puncturing a desired tissue or membrane (e.g., the retina) to reduce the risk of tissue damage as caused by unintended movement or tremor of the user. Extension and retraction of the injection needle 712 to adjust needle stiffness may be controlled by any suitable mechanisms, including those described in FIGS. 5 and 6.

The annular insert 730 is generally formed of any suitable surgical-grade materials, such a metallic or thermoplastic polymeric materials that facilitate the extension and retraction of the injection needle 712 from/to the injection cannula 710. Examples of metallic materials include aluminum, stainless steel, and other metallic alloys. Examples of suitable thermoplastic polymeric materials include polyether ether ketone (PEEK), polyetherketone (PEK), and polytetrafluoroethylene (PTFE).

As shown in FIGS. 7A and 7B, the annular insert 730 may be fixedly coupled to an inner wall 708 of the injection cannula 710 and thus, have an outer diameter 730_{OD} substantially matching the inner diameter 710_{ID} of the injection cannula 710. In certain embodiments, a distal surface 732 of the annular insert 730 is flush with a distal surface 740 of the injection cannula 710. To facilitate the extension and retraction of the injection needle 712 from/to the injection cannula 710 while also reducing the air gap between the injection needle 712 and the injection cannula 710, the annular insert 730 may have an inner diameter 730_{ID} substantially matching the outer diameter of the injection needle 712. For example, the inner diameter 730_{ID} may be equal to or approximately equal to an outer diameter of a 38-gauge needle.

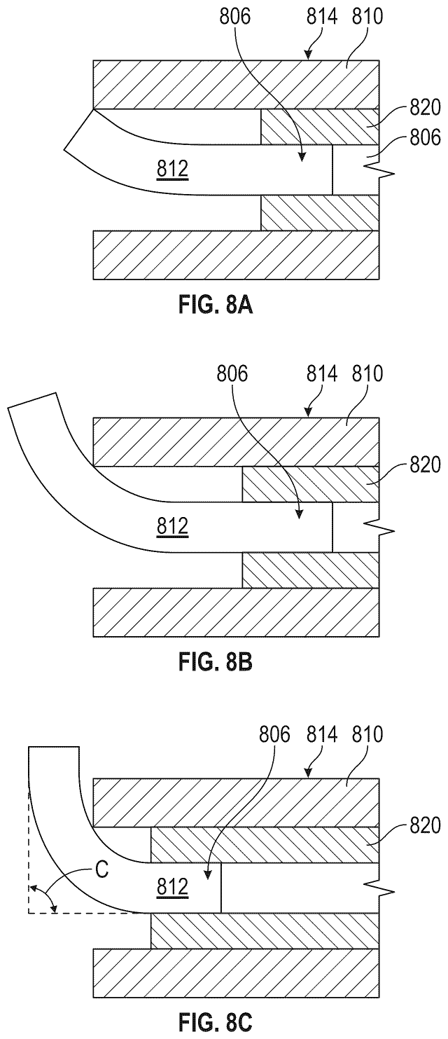

FIGS. 8A-8C illustrate schematic, cross-sectional side views of another distal end 814 of an exemplary injection cannula 810, according to certain embodiments of the present disclosure. The injection cannula 810 is an exemplary injection cannula that may be utilized with the delivery devices 500 and 600 of FIGS. 5 and 6, or other delivery devices for subretinal injection as described herein. Aspects of the injection cannula 810 may be combined with other delivery devices and/or components described herein without limitation.

As shown, an injection needle 812 is disposed within the tubular injection cannula 810 and is configured to slidably extend from and retract into the distal end 814 thereof. Similar to the embodiments in FIGS. 7A and 7B, a proximal end 806 of the injection needle 812 is coupled to an inner fluidic shaft 820 for fluidic coupling between the injection needle 812 and flexible fluidic tubing connected to a fluid source. In such embodiments, the inner fluidic shaft 820 may be slidably disposed within the cannula 810 to facilitate extension and retraction of the injection needle 812. However, in certain embodiments, the injection needle 812 may be coupled directly to the fluidic tubing.

In FIGS. 8A-8C, the injection needle 812 comprises a pre-shaped and curved needle formed of an elastic (or flexible) material. In certain embodiments, the injection needle 812 may be formed of a superelastic material such as nitinol. The utilization of a curved and elastic material enables an adjustable insertion angle for the injection needle 812 during performance of subretinal injections, thereby facilitating easier positioning into the subretinal space, as well as easier access to peripheral areas of the retina not normally accessible by straight injection needs. In certain examples, the adjustable insertion angle of the injection needle 812 facilitates reduced damage of tissues underlying the subretinal space, such as the retinal pigment epithelium (RPE), since the subretinal space may be entered at a lower angle, thereby improving the safety of subretinal injections.

In certain embodiments, the insertion angle (or curvature) of the injection needle 812 may be adjusted, or controlled, by extending or retracting the injection needle 812 from/to the injection cannula 810. In certain embodiments, the curvature of the injection needle 812 may be increased by extending the injection needle 812 from the injection cannula 810, as shown in FIGS. 8A-8C. Note that in the embodiments of FIGS. 8A-8C, to facilitate bending or curving of the injection needle 812 when extended from the injection cannula 810, the injection cannula 810 may not comprise an annular insert at a distal end 814 thereof. However, in certain embodiments, an annular insert similar to annular insert 730 may be utilized.

Referring to FIG. 8A, in a retracted state, the injection needle 812 may be substantially straight, or only slightly curved, as curvature thereof is limited by the inner diameter of the injection cannula 810. In FIG. 8B, the injection needle 812 is partially extended from the injection cannula 810, and starts increasing in curvature as it exits the injection cannula 810. In FIG. 8C, the injection needle 812 is fully extended from the injection cannula 810, and is disposed at a maximum curvature as the elastic material thereof deforms back to its original shape. Thus, in the examples shown, the greater the distance the injection needle 812 is extended out of the injection cannula 810, the greater an angle of curvature C the injection needle 812 assumes. In certain embodiments, a maximum angle of curvature C of the injection needle 812 is 90° relative to a major longitudinal axis of the injection cannula 810.

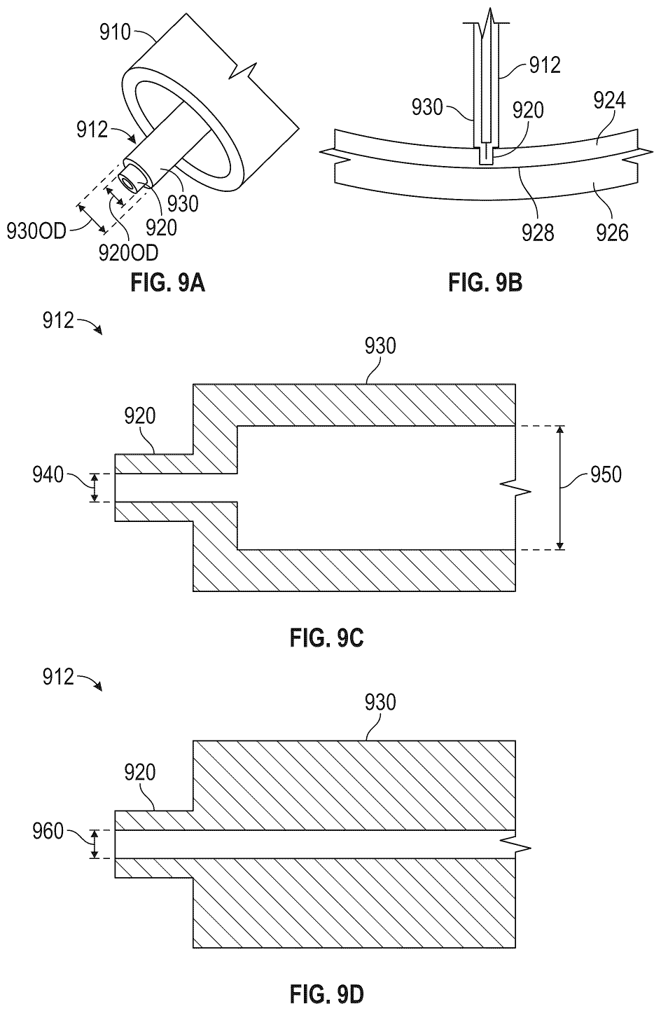

FIGS. 9A-9D illustrate various views of an exemplary injection needle 912, according to certain embodiments of the present disclosure. The injection needle 912 is an exemplary injection needle that may be utilized with any of the injection cannulas and/or delivery devices for subretinal injection as described herein. Thus, aspects of the injection needle 912 may be combined with other delivery devices and/or components described herein without limitation. For illustrative purposes, the injection needle 912 is shown disposed within a tubular injection cannula 910.

As shown, the injection needle 912 comprises a stepped needle. In other words, the injection needle 912 comprises two or more portions having differing outer diameters, wherein the outer diameters get progressively larger in a stepwise (i.e., incremental) fashion along a length of the injection needle 912 in a proximal direction. In certain embodiments, as in FIGS. 9A-9D, the injection needle 912 comprises a first, distal portion 920 having a first outer diameter 920_{OD}, and a second, proximal portion 930 having a second outer diameter 930_{OD}. In such embodiments, the outer diameter 930_{OD} of the proximal portion 930 is larger than the outer diameter 920_{OD} of the distal portion 920. For example, the distal portion 920 may have a gauge of 38, and the proximal portion 930 may have a gauge of 37, 36, 35, 34, 33, 32, 31, 30, or more. In another example, the distal portion 920 may have a gauge of 41, and the proximal portion 930 may have a gauge of 40, 39, 38, 37, 36, 35, 34, 33, or more. In another example, the distal portion 920 may have a gauge of 41, and the proximal portion 930 may have a gauge of 38 or larger.

The stepped outer morphology of the injection needle 912 facilitates improved safety during performance of subretinal injections by ensuring the injection needle 912 does not pass through the subretinal space and into underlying tissues, thereby damaging such tissues. For example, as shown in FIG. 9B, when performing a subretinal injection using a transvitreal approach, the proximal portion 930 may act as a mechanical stop and prevent the distal portion 920 of the injection needle 912 from passing through the subretinal space 928 and puncturing the RPE 926. Accordingly, in such embodiments, the distal portion 920 may have a length along a major longitudinal axis of the injection needle 912 that corresponds to a thickness of the retina 924, and the proximal portion 930 may have a length along the major axis of the injection needle 912 that corresponds to the remaining length of the injection needle 912. Additionally, the stepped outer morphology of the injection needle 912 (e.g., the wider proximal portion 930) may prevent fluids injected into the subretinal space 928 from leaking from, or escaping, the subretinal space 928 through the puncture wound formed by the injection needle 912.

In certain embodiments, in addition to having a stepped outer morphology, the injection needle 912 may have a stepped inner morphology. For example, as shown in FIG. 9C, the injection needle 912 may comprise a first, smaller inner diameter 940 substantially corresponding with the distal portion 920, and a second, larger inner diameter 950 substantially corresponding with the proximal portion 930. The utilization of a stepped inner diameter may reduce the risk or occurrence damage to tissues surrounding the subretinal space during injection of fluids (e.g., the RPE), as the transition of the larger inner diameter 950 to the smaller inner diameter 940 creates reduced fluidic resistance, thereby reducing the overall force of fluidic jet streams dispensed by injection needle 912. In certain other embodiments, however, the injection needle 912 may comprise a single inner diameter 960 through a length of the injection needle 912, as shown in FIG. 9D.

In certain embodiments, the stepped morphology of the injection needle 912 is formed by shrinking or pressing the distal portion 920 into a desired shape/dimension. In certain embodiments, the stepped morphology of the injection needle 912 is formed by expanding the proximal portion 930 into a desired shape/dimension. In certain embodiments, the stepped morphology of the injection needle 912 is formed by assembling two separate tubes together by any suitable techniques, including welding or the use of adhesives.

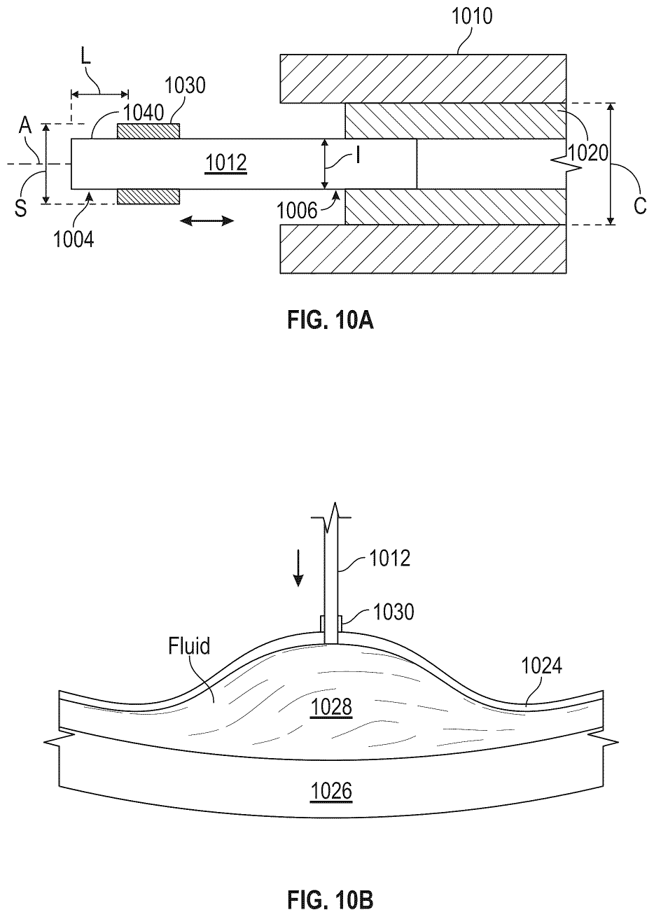

FIGS. 10A-10B illustrate various views of another injection needle 1012, according to certain embodiments of the present disclosure. The injection needle 1012 is an exemplary injection needle that may be utilized with any of the injection cannulas and/or delivery devices described herein. Thus, aspects of the injection needle 1012 may be combined with other delivery devices and/or components described herein without limitation. For illustrative purposes, the injection needle 1012 is shown disposed within a tubular injection cannula 1010 and coupled to inner fluidic shaft 1020 within the injection cannula 1010 at a proximal end 1006 of injection needle 1012.

As shown in FIG. 10A, the injection needle 1012 comprises a sealing element 1030 disposed around a portion of a distal end 1004 thereof. In certain embodiments, the sealing element 1030 comprises an annular ring formed around (circumscribing) the injection needle 1012. However, other morphologies are also contemplated. Generally, the sealing element 1030 has on outer dimension S that is greater than an outer dimension I of the injection needle 1012, but less than an inner diameter C of the injection cannula 1010, such that the sealing element 1030 may fit within the injection cannula 1010 in embodiments where the injection needle 1012 is configured to extend from/retract into the injection cannula 1010. In certain embodiments, the sealing element 1030 is formed of a flexible, elastic, or supple material with sealing qualities to prevent damage to tissues contacted thereagainst. For example, the sealing element 1030 may comprise a silicone or rubber-based material.

In certain embodiments, a segment 1040 of the injection needle 1012 distal to the sealing element 1030 may have a length L along a major longitudinal axis A of the injection needle 1012 that corresponds to a thickness of the retina (labeled 1024 in FIG. 10B). This length L, in combination with the increased outer dimension(s) of the sealing element 1030 relative to the injection needle 1012, ensures that the injection needle 1012 does not pass through the subretinal space (labeled 1028 in FIG. 10B) and into underlying tissues, such as the RPE (labeled 1026 in FIG. 10B), during injection. Accordingly, in such examples, the sealing element 1030 functions as a mechanical stop similar to the proximal portion of the stepped injection needle in FIGS. 9A-9C, and reduces the risk of damage to tissues underlying the subretinal space 1028.

In certain embodiments, the sealing element 1030 may prevent fluids injected into the subretinal space 1028 from leaking from, or escaping, the subretinal space 1028 through the puncture wound formed by the injection needle 1012. For example, as shown in FIG. 10B, the sealing element 1030 may prevent injected fluids from escaping through the retina 1024.

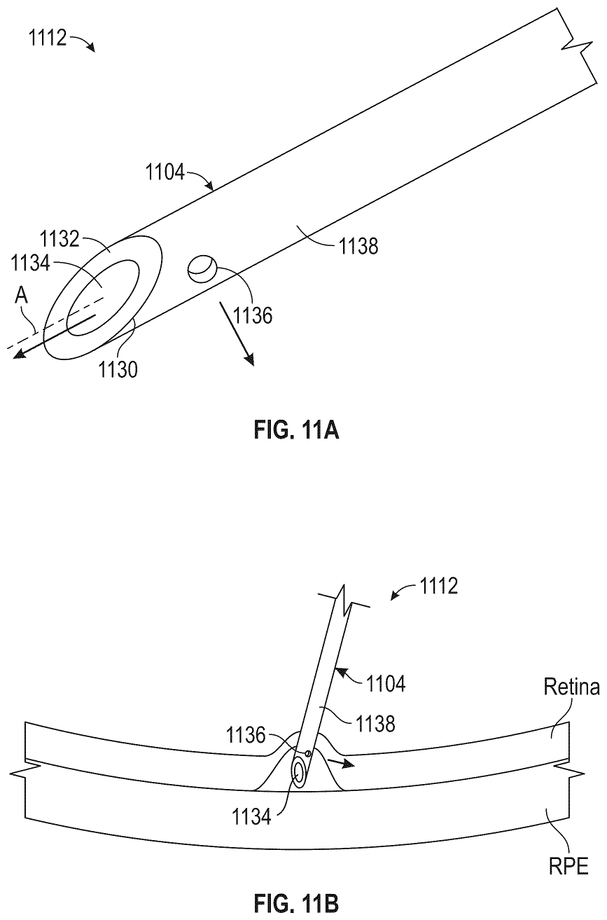

FIGS. 11A-11B illustrate various views of another injection needle 1112, according to certain embodiments of the present disclosure. The injection needle 1112 is an exemplary injection needle that may be utilized with any of the injection cannulas and/or delivery devices for subretinal injection as described herein. Aspects of the injection needle 1112 may therefore be combined with other delivery devices and/or components described herein without limitation.

As shown in FIG. 11A, the injection needle 1112 comprises a beveled tip 1130 with a port 1134 at a distal end 1104 thereof. At least a portion of an endface 1132 of the beveled tip 1130 is beveled, or angled, at a non-orthogonal angle relative to a major longitudinal axis A of the injection needle 1112. That is, some or all of the endface 1132 is non-planar with a plane normal to the major axis A of the injection needle 1112. In certain embodiments, some or all of the endface 1132 of the injection needle 1112 is disposed at an angle between about 0° and about 900 relative to the plane normal to the major axis A, such between about 30° and about 60° to such plane. In certain embodiments, the endface 1132 is planar. In some embodiments, the endface 1132 is curved, or comprises two or more nonplanar portions. The beveled tip 1130 provides reduced traction and easier puncturing of tissues, such as the retina, to reach the subretinal space during performance of a subretinal injection. Thus, the beveled tip 1130 facilitates reduced tearing of ocular tissues during injections, thereby improving the safety of such procedures as compared to utilization of other tip morphologies.

In certain embodiments, the injection needle 1112 further comprises a side port 1136 disposed through a sidewall 1138 of the injection needle 1112. While the port 1134 through the beveled tip 1130 functions as a main outlet for egress of injection fluids, the side port 1136 may serve as a secondary outlet for such injection fluids. Accordingly, the inclusion of the side port 1136 facilitates a reduced fluidic jetstream of injection fluids through/from port 1134 during injection, which may be disposed adjacent to and/or facing one or more tissues during the injection. For example, during a transvitreal subretinal injection, the port 1134 may be positioned adjacent to and facing the RPE in the subretinal space. Thus, upon injection, injected fluid will be directed at the RPE, which may cause damage thereto if injected with too much force. By including the side port 1136, a portion of the injection fluid is directed/flowed peripherally, thereby reducing the fluidic jetstream directed at the RPE and minimizing any damage caused thereby.

In further embodiments, the injection needle 1112 may not comprise the port 1134 through the beveled tip 1130, and may instead only comprise the side port 1136 as an outlet for injection fluids. In such embodiments, the injection needle 1112 may be referred to as a “closed” needle, as the beveled tip 1130 may comprise a solid, closed endface 1132.

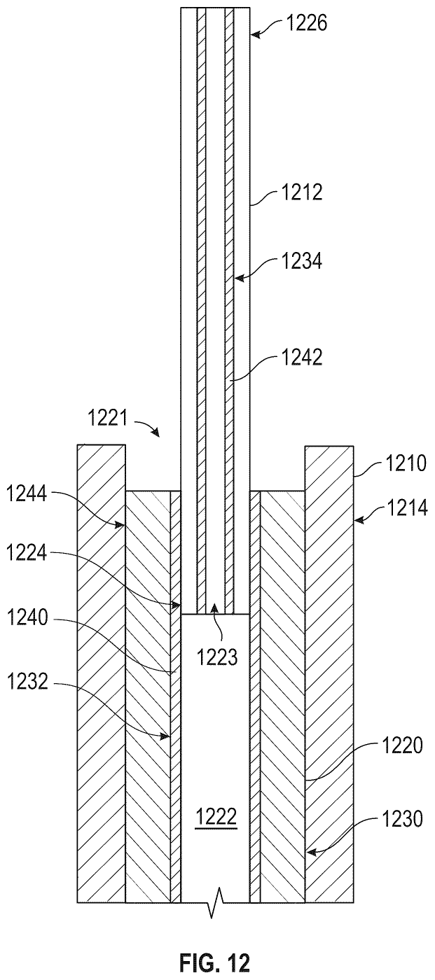

FIG. 12 illustrates a schematic, cross-sectional side view of a distal end 1214 of an exemplary injection cannula 1210, according to certain embodiments of the present disclosure. The injection cannula 1210 is an exemplary tubular injection cannula that may be utilized with any of the delivery devices for subretinal injection as described herein. Aspects of the injection cannula 1210 may therefore be combined with other delivery devices and/or components described herein without limitation.

As shown, an injection needle 1212 is disposed within the injection cannula 1210 and is coupled to an inner fluidic shaft 1220 extending along a length of an inner channel 1221 of the injection cannula 1210. The inner channel 1221 of the injection cannula 1210 extends from a proximal end of the injection cannula 1210 to the distal end 1214. The injection needle 1212 and inner fluidic shaft 1220 are configured to slidably extend from and retract into the distal end 1214, for example, upon actuation of a toggle operably coupled to the inner fluidic shaft 1220. In other embodiments, however, the injection needle 1212 may be directly and slidably coupled to the injection cannula 1210 without the inner fluidic shaft 1220. In such embodiments, the injection needle 1212 may extend along an entirety of the length of the inner channel 1221.

Like the injection cannula 1210, the inner fluidic shaft 1220 and injection needle 1212 comprise their own inner channels 1222 and 1223, respectively. In the example of FIG. 12, the inner channel 1222 extends from a proximal end of the inner fluidic shaft 1220 to a distal end 1244 of the inner fluidic shaft 1220, and the inner channel 1223 extends from a proximal end 1224 of the injection needle 1212 to a distal end 1226 thereof. During performance of a subretinal injection, injection fluids (e.g., a non-treatment and/or a treatment solution) from a fluid source in fluid communication with the injection needle 1212 are flowed through the inner channels 1221, 1222, and/or 1223, and are dispensed from the distal end 1226 of injection needle 1212.

Each inner channel 1221, 1222, and 1223 is at least partially defined by an inner wall 1230, 1232, or 1234 of the injection cannula 1210, inner fluidic shaft 1220, and injection needle 1212, respectively. In the embodiment of FIG. 12, the inner walls 1232 and 1234 have a coating 1240 or 1242 disposed thereon, respectively. The coatings 1240 and 1242 are configured to reduce surface adhesion and/other effects of the surfaces of inner walls 1232 and 1234 on injection fluids flowed through the inner fluidic shaft 1220 and injection needle 1212. Accordingly, the coatings 1240 and 1242 facilitate lower fluidic resistance through the inner fluidic shaft 1220 and injection needle 1212, thereby allowing a lower pressure to be applied to generate the necessary fluidic flow for a subretinal injection.

In certain embodiments, the coating 1240 and/or 1242 comprises a polymer brush coating, such as a polymer brush coating formed of poly(ethylene oxide) (PEO), poly(methyl methacrylate) (PMMA), poly(hydroxyethyl methacrylate) (PHEMA), combinations thereof, and the like. In certain embodiments, the coating 1240 and/or 1242 includes a fluoropolymer coating, such as a coating formed of polytetrafluoroethylene (PTFE), ethylene tetrafluoroethylene (ETFE), perfluoroalkoxy (PFA), chlorotrifluoroethylene (E-CTFE), combinations thereof, and the like. In certain embodiments, the coating 1240 and/or 1242 comprises a polyether ether ketone (PEEK) coating. Other functionalized coatings are also contemplated. Generally, the coating 1240 and/or 1242 has a thickness between about 1 nm (nanometer) and about 1000 nm, such as between about 1 nm and about 500 nm, such as between about 1 nm and about 100 nm. In certain embodiments, the coatings 1240 and 1242 are substantially the same. For example, the coatings 1240 and 1242 may comprise the same type, material, thickness, etc. In certain other embodiments, the coatings 1240 and 1242 are different. For example, the coatings 1240 and 1242 may comprise a different type, material, thickness, etc.

Note that wherein the injection needle 1212 is directly coupled to the injection cannula 1210 without the inner fluidic shaft 1220, the coating 1240 may be disposed along the inner wall 1230 of the injection cannula 1210.

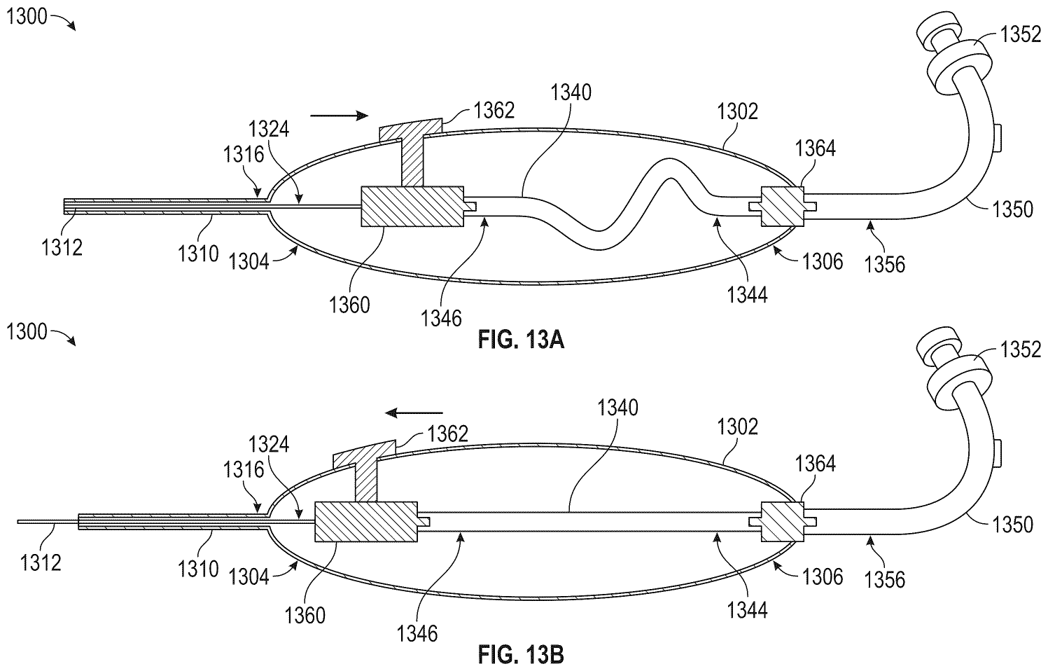

Referring now to FIGS. 13A-13B, another exemplary subretinal delivery device 1300 is illustrated in schematic cross-sectional side views according to certain embodiments of the present disclosure. The delivery device 1300 may be used as, e.g., the delivery device 414 of the surgical system 400 of FIG. 4, and in combination with any of the injection cannulas, injection needles, or other components described herein without limitation.

The delivery device 1300 includes a handle 1302 and a tubular injection cannula 1310 having a proximal end 1316 coupled to and extending distally from a distal end 1304 of the handle 1302, and a curved or substantial straight injection needle 1312 disposed within the injection cannula 1310 (a straight injection needle 1312 is shown) and configured to slidably extend from and retract into the injection cannula 1310 by actuation of a toggle 1362. In certain embodiments, the injection needle 1312 is coupled to an inner fluidic shaft at least partially disposed within the cannula 1310 for fluidic coupling between the injection needle 1312 and the toggle 1362 or fluidic tubing. In such embodiments, the inner fluidic shaft may be slidably disposed within the cannula 1310 to facilitate extension and retraction of the injection needle 1312 upon actuation of the toggle 1362. In certain embodiments, the handle 1302 is rotatable, as described above with reference to FIG. 6.

The injection needle 1312 is fluidically coupled, directly or indirectly, at a proximal end 1324 thereof to a distal end 1346 of a flexible first fluidic tubing 1340 disposed within the handle 1302. In certain embodiments, the first fluidic tubing 1340 if fabricated of silicone, thermoplastic polyurethane (TPU), combinations thereof, or other flexible materials. A proximal end 1344 of the first fluidic tubing 1340 terminates at, or substantially near, a proximal end 1306 of the handle 1302, where the first fluidic tubing 1340 is fluidically coupled, directly or indirectly, to a distal end 1356 of a flexible second fluidic tubing 1350 having a proximal connector 1352 for coupling to a fluid source. Similar to the first fluidic tubing 1340, in certain embodiments, the second fluidic tubing 1350 if fabricated of silicone, thermoplastic polyurethane (TPU), combinations thereof, or other flexible materials. The second fluidic tubing 1350 is configured as a supply line to supply injection fluids, e.g., non-treatment and/or a treatment solutions, from a fluid source to the delivery device 1300, and more particularly, the first fluidic tubing 1340 and injection needle 1312. In certain embodiments, the fluid source comprises a fluidic system, which may be coupled to the second fluidic tubing 1350 via a connection 1364, such as a Luer lock or other male-female coupling. In operation, the first fluidic tubing 1340 facilitates decoupling of the injection needle 1312 from the second fluidic tubing 1350, thereby causing any mechanical stresses (e.g., pulling) on the second fluidic tubing 1350 to stop at the handle 1302. This prevents such mechanical stresses from acting on the injection needle 1312, which could otherwise cause the injection needle 1312 to be involuntarily extracted from or retracted into the injection cannula 1310.

In certain embodiments, the first fluidic tubing 1340 and the second fluidic tubing 1350 are of a same type, material, diameter, etc. In certain other embodiments, the first fluidic tubing 1340 and the second fluidic tubing 1350 are of a different type, material, diameter, etc. For example, in certain embodiments, the first fluidic tubing 1340 may comprise a more pliable/flexible material as compared to the second fluidic tubing 1350. In certain embodiments, the first fluidic tubing 1340 has a smaller or larger diameter as compared to the second fluidic tubing 1350.

In the embodiments depicted in FIGS. 13A-13B, a base 1360 of a lockable toggle 1362 fluidically couples the proximal end 1324 of the injection needle 1312 to the distal end 1346 of the first fluidic tubing 1340. However, other coupling arrangements and/or mechanisms between the injection needle 1312 and the first fluidic tubing 1340 are contemplated. Similarly, the proximal end 1344 of the first fluidic tubing 1340 is indirectly but fluidically coupled to the distal end of the second fluidic tubing 1350 via a connection 1364 fixedly disposed at the proximal end 1306 of the handle 1302. The connection 1364 may comprise any suitable type of connector, such as a male-male coupling. Again, other coupling arrangements and/or mechanisms between the first fluidic tubing 1340 and the second fluidic tubing 1350 are contemplated.

The toggle 1362 can be manually controlled by a user to cause actuation of the injection needle 1312, e.g., extension or retraction into/from the injection cannula 1310, via any of the toggle mechanisms described herein. As described above, the toggle 1362 is lockable, and therefore upon release of the toggle 1362, the toggle 1362 is locked into place. Accordingly, a user may adjust the position of the injection needle 1312 via manual actuation of the toggle 1362, and may then lock the injection needle 1312 in place by releasing the toggle 1362. Because the injection needle 1312 is decoupled from the external second fluidic tubing 1350, however, movement and/or positioning of the injection needle 1312 of subretinal delivery device 1300 remains unaffected by the second fluidic tubing 1350. Thus, movement of the second fluidic tubing 1350 has no effect on needle position (or vice-versa), and will not disrupt or unwantedly cause repositioning of the injection needle 1312 after the user has positioned/locked the injection needle 1312 using the toggle 1352.

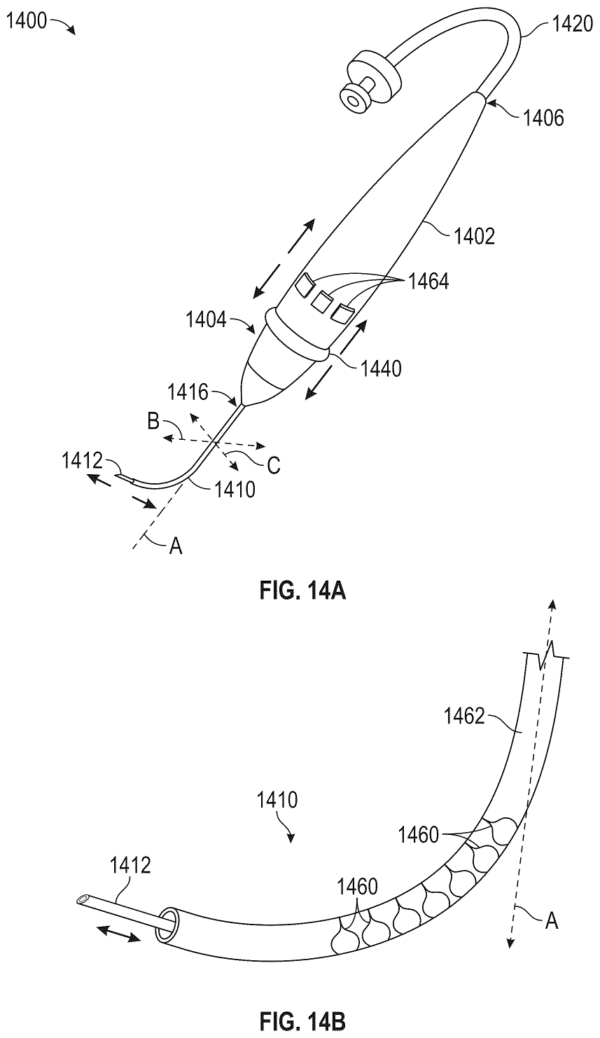

FIGS. 14A-14B illustrate perspective side views of an exemplary subretinal delivery device 1400 having an articulating and tubular injection cannula 1410, according to certain embodiments of the present disclosure. The delivery device 1400 with the injection cannula 1410 may be used as, e.g., the delivery device 414 of the surgical system 400 of FIG. 4 or other surgical systems for subretinal injection as described herein. Aspects of the delivery device 1400 may be combined with other delivery devices and/or components described herein without limitation.

As shown in FIG. 14A, the delivery device 1400 further includes a handle 1402, wherein a proximal end 1416 of the injection cannula 1410 is coupled to and extends distally from a distal end 1404 of the handle 1402. Within the injection cannula 1410 is a curved or substantial straight injection needle 1412 (a straight injection needle 1412 is shown) that is configured to slidably extend from and retract into the injection cannula 1410 by actuation of a toggle 1440. In certain embodiments, the injection needle 1412 is coupled to an inner fluidic shaft at least partially disposed within the cannula 1410 for fluidic coupling between the injection needle 1412 and the toggle 1440 or fluidic tubing. In such embodiments, the inner fluidic shaft may be slidably disposed within the cannula 1410 to facilitate extension and retraction of the injection needle 1412 upon actuation of the toggle 1440. In certain embodiments, the handle 1402 is rotatable, as described above with reference to FIG. 6.

In certain embodiments, a flexible fluidic tubing 1420 for supplying injection fluids (e.g., non-treatment and/or a treatment solutions) to the delivery device 1400 may be disposed through a proximal end 1406 of the handle 1402 and fluidically coupled to the injection needle 1412 within the handle 1402. Alternatively, the fluidic tubing 1420 may couple to the proximal end 1406 of the handle 1402, or another fluidic tubing within the handle 1402. In certain embodiments, the fluidic tubing 1420 comprises a multi-lumen tubing which provides a plurality of parallel flow paths from separate fluid reservoirs of the fluid source to the injection needle 1412 so that an injection can be performed using only one needle.

The injection cannula 1410, which may comprise a tube, and/or the injection needle are generally formed of any suitable surgical-grade materials, such as metallic or thermoplastic polymeric materials. Examples of metallic materials include aluminum, stainless steel, and other metallic alloys. Examples of suitable thermoplastic polymeric materials include polyether ether ketone (PEEK), polyetherketone (PEK), and polytetrafluoroethylene (PTFE).

In the examples of FIGS. 14A and 14B, the injection cannula 1410 is controllably articulable. In other words, the injection cannula 1410 is controllably bendable by manual adjustment of the user. To facilitate the articulating nature of the injection cannula 1410, the injection cannula 1410 may comprise a plurality of features 1460 formed in an outer surface 1462 of the injection cannula 1410, which enable flexibility of the injection cannula 1410 in one or more directions orthogonal to a major longitudinal axis A of the injection cannula 1410. For example, in certain embodiments, the features 1460 may be formed in the outer surface 1462 such that the injection cannula 1410 is articulable in opposing directions for one or both perpendicular axes B and C disposed along a plane perpendicular to the major axis A. In such examples, two or more sets of features 1460 may be formed on injection cannula 1410 on opposing surfaces of the outer surface 1462. In certain embodiments, the features 1460 comprise, e.g., slots, crevices, or other suitable features etched or cut (e.g., laser etched or cut) into the outer surface 1462 along a length of the injection cannula 1410 to facilitate biasing of the injection cannula 1410 into a curved position.

Articulation, or bending, of the injection cannula 1410 may be manually controlled by one or more toggles 1464 separate from the toggle 1440. In certain embodiments, the one or more toggles 1464 may be the same type of toggle as toggle 1440. In certain embodiments, the one or more toggles 1464 may be a different type of toggle as compared to toggle 1440. In FIG. 14A, a plurality of toggles 1464 are disposed around a circumference of handle 1402. In such embodiments, each of the plurality of toggles 1464 may control bending of the injection cannula 1410 in a different direction. Generally, the one or more toggles 1464 may be coupled to one or more wires disposed within the handle 1402 and coupled to different points within an interior of the injection cannula 1410 which, when manipulated by user adjustment of the toggles 1464, acts on the injection cannula 1410 to cause the injection cannula 1410 to articulate in a corresponding direction. Manipulation of the wires may thus create a curvature of the injection cannula 1410 in a desired direction.

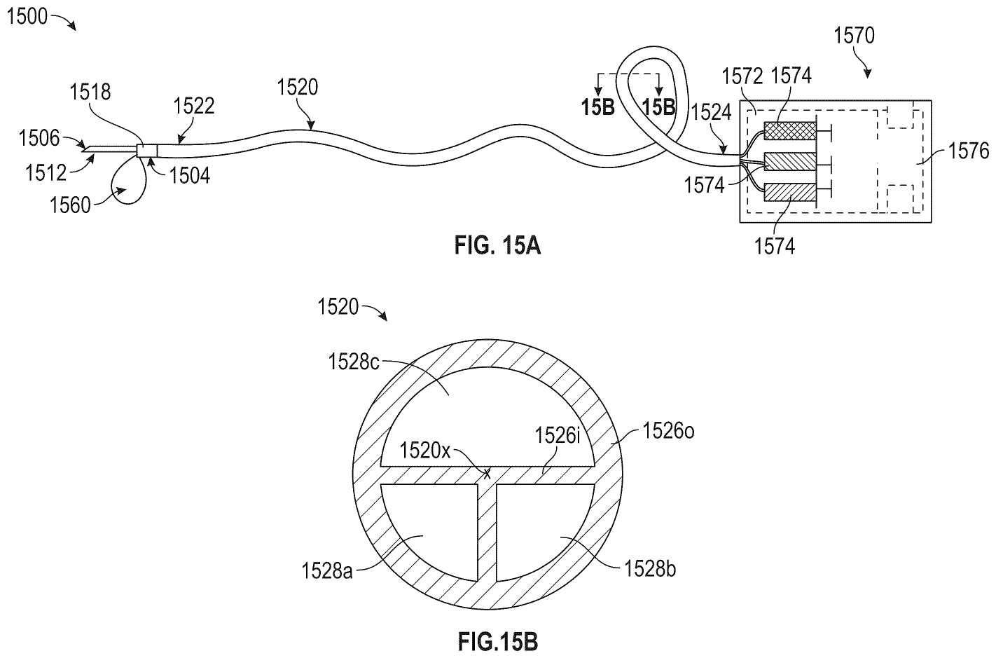

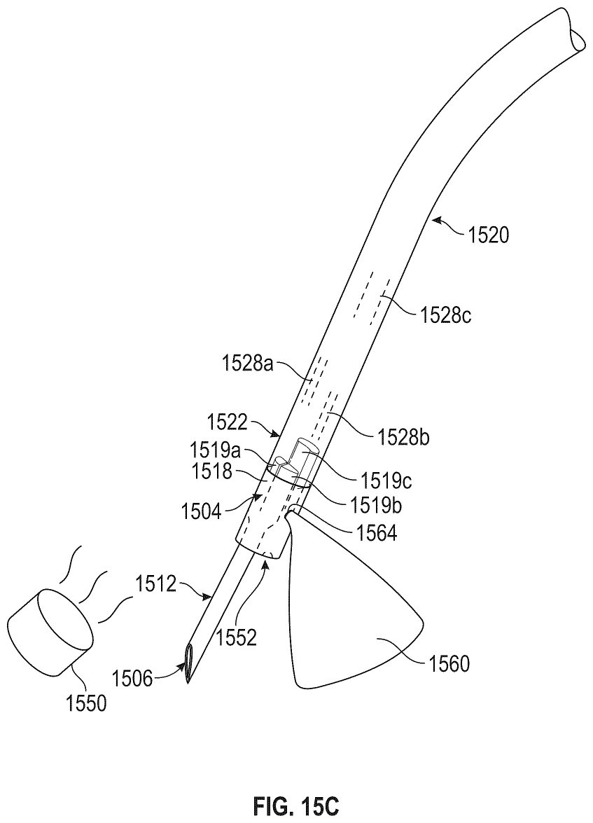

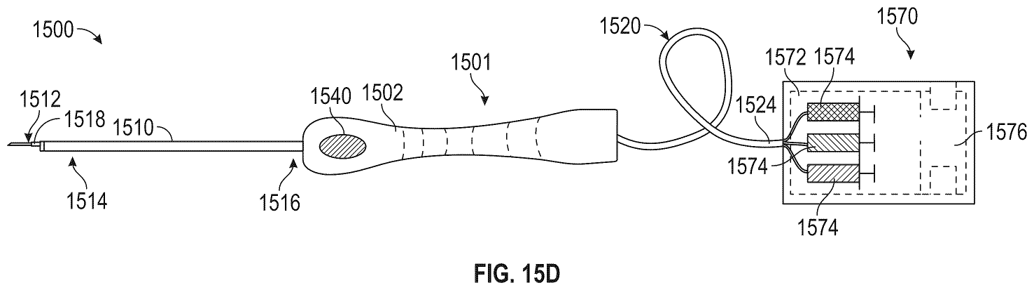

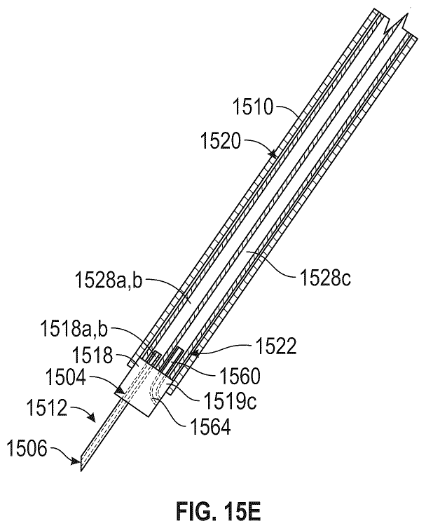

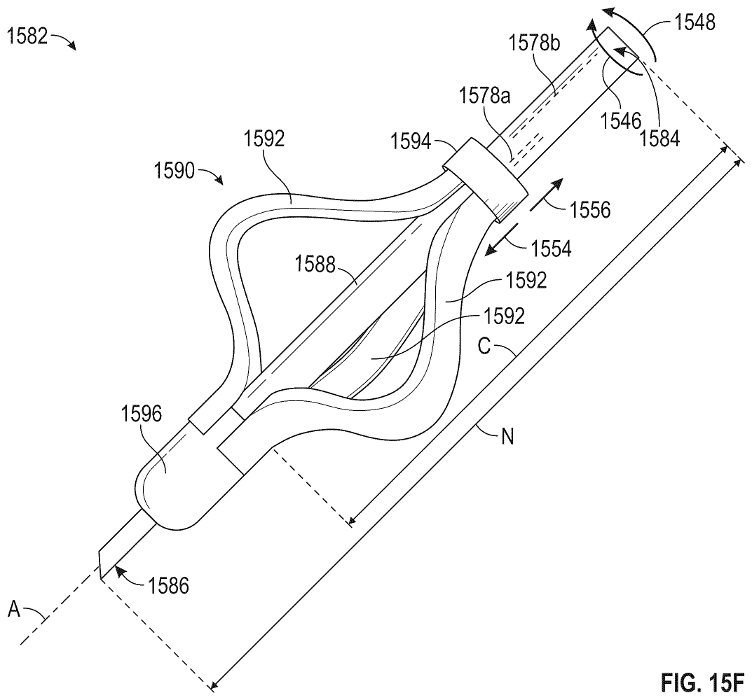

FIG. 15A illustrates a schematic view of an exemplary subretinal delivery system 1500, according to certain embodiments of the present disclosure. The delivery system 1500 may be used with, e.g., the delivery device 414 of the surgical system 400 of FIG. 4 or other surgical systems for subretinal injection as described herein. Aspects of the delivery system 1500 may be combined with other delivery devices and/or components described herein without limitation.

In general, the delivery system 1500 includes an injection needle attached to a tubing, which can be decoupled from a handle and secured within the eye using a stabilizer such that the injection instrument does not need to be held throughout the entire procedure. This provides decoupling of the injection needle from undesirable movements that would otherwise occur when the injection instrument is handheld. Furthermore, the tubing of the delivery system 1500 may, in certain examples, be a multi-lumen tubing, which provides a plurality of parallel flow paths from separate fluid reservoirs to the injection needle so that the injection can be performed using only one needle. Having to insert only one needle through the retina can reduce damage to the retina, which could otherwise occur from repeated piercing of the retina.

As shown in FIG. 15A, the delivery system 1500 generally includes an injection needle 1512, a single or multi-lumen tubing 1520, a stabilizer 1560, and a fluidic drive system 1570. The injection needle 1512 has a proximal end 1504 and a distal end 1506. In certain embodiments, the injection needle 1512 further includes a connector piece 1518 (described in more detail below) at the proximal end 1504 that facilitates connections of the injection needle 1512 to the tubing 1520. The tubing 1520 has a distal end 1522 attached to the proximal end 1504 of the injection needle 1512 through the connector piece 1518 and a proximal end 1524 attached to the fluidic drive system 1570 (e.g., through a handle as discussed below).

FIG. 15B is an enlarged cross-sectional view taken along the section line 15B-15B of FIG. 15A illustrating an exemplary tubing 1520 having multiple lumens, which also be utilized with other delivery devices and systems described herein. The multi-lumen tubing 1520 includes an outer wall 1526o surrounding three lumens 1528a, 1528b, and 1528c. Although FIG. 15B shows three lumens, more or less lumens can be used (e.g., two or more lumens, from two to four lumens, two lumens, or four lumens). The lumens 1528a-c are divided by inner walls 1526i intersecting the outer wall 1526o. The lumens 1528a-c are radially surrounding a center longitudinal axis 1520x of the tubing 1520. In the embodiments of FIG. 15B, one or more of the lumens 1528a-c have different sizes. For example, each of the lumens 1528a, 1528b extend one-quarter of the way around the tubing 1520 in a circumferential direction. On the other hand, the lumen 1528c extends halfway around tubing 1520 in the circumferential direction. Therefore, in the embodiments of FIG. 15B, a volume of the lumen 1528c may be twice as much as a volume of each of the lumens 1528a, 1528b. In some other embodiments, each of the lumens 1528a-c has the same size.

Returning now to FIG. 15A, the fluidic drive system 1570 may comprise a fluid pump 1572 for driving flow through the tubing 1520. In certain embodiments, the fluid pump may comprise a syringe pump, a Vernier Flow Control (VFC) pump, or another type of pressure control pump, a volume control pump, a variable volume control pump, a peristaltic pump, a lever-actuated pump, a valve-actuated pump, or a venturi pump. The fluidic drive system 1570 further comprises one or more fluid reservoirs 1574 for storing one or more fluids for injection. Although FIG. 15A shows three fluid reservoirs 1574, more or less fluid reservoirs can be used. In certain embodiments, each of the fluid reservoirs 1574 may be configured to be actuated by the fluid pump 1572 to drive flow of fluids stored therein. Such fluids may comprise injection fluids, including: a non-treatment solution, such as an ophthalmic irrigation solution having physiological pH (potential of hydrogen) and osmotic pressure (e.g., BSS); and a treatment solution, such as a therapeutic substance for treating the eye (e.g., anti-VEGF (vascular endothelial growth factor)), tissue plasminogen activator (tPA), stem cells, viral vectors for gene therapy, other drugs, or combinations thereof). The fluids may also comprise a working fluid, such as a fluid for extending the stabilizer 1560 (e.g., perfluorocarbon liquid (PFCL), BSS, saline, air, N2 (Nitrogen), other liquids or gases, or combinations thereof).

The fluidic drive system 1570 further comprises a controller 1576 for controlling operation of the fluid pump 1572. In certain embodiments, the controller 1576 includes a wireless receiver receiving instructions wirelessly from a control console, e.g., surgical console 402. In certain other embodiments, the controller 1576 is wiredly in communication with a control console.

FIG. 15C is top isometric view of a portion of the delivery system 1500 of FIG. 15A. As shown, the connector piece 1518 has one or more ports 1519 corresponding to the one or more lumens of the tubing 1520. In the example of FIG. 15C, three ports 1519a, 1519b, 1519c are shown, each corresponding to and/or disposed within the distal ends of the lumens 1528a-c, respectively, of the multi-lumen tubing 1520 in FIG. 15B. The two separate ports 1519a, 1519b of the connector piece 1518 merge together toward the distal end 1506 of the injection needle 1512. The port 1519c, on the other hand, is separate from each of the ports 1519a, 1519b and is fluidly isolated therefrom. The port 1519c is fluidly coupled to the stabilizer 1560 as shown for facilitating extension thereof.

Referring to FIGS. 15A and 15C, the stabilizer 1560 is shown in the extended position where the stabilizer 1560 extends from the port 1519c of the connector piece 1518. In certain embodiments, the stabilizer 1560 and port 1519c are oriented such that the stabilizer 1560 extends distally from a distal end of the connector piece 1518, as shown in FIG. 15C. In certain embodiments, the stabilizer 1560 and port 1519c are oriented such that the stabilizer 1560 extends laterally through an external port 1564 in a sidewall of the connector piece 1518. In the extended position, the stabilizer 1560 stabilizes/immobilizes the injection needle 1512 at a target injection site (i.e., position) on the retinal surface for subretinal injection, which reduces the likelihood of the injection needle 1512 being removed from the subretinal space during the procedure due to light inadvertent forces. The stabilization further controls the location of injection. In the embodiments of FIGS. 15A and 15C, the stabilizer 1560 is a balloon 1562, or bag. The balloon 1562 can have any suitable shape including without limitation, round, oval, or polygonal. The balloon 1562 may be formed from plastic, metal, polymer, nitinol, or combinations thereof.

Before the stabilizer 1560 is in the extended position, the stabilizer 1560 is disposed within the port 1519c of the connector piece 1518. In certain embodiments, to actuate the stabilizer 1560 to the extended position, a working fluid (e.g., PFCL) is injected from one of the fluid reservoirs 1574 of the fluidic drive system 1570 through a lumen, e.g., lumen 1528c, to fill the balloon 1562. In certain embodiments, the balloon 1562, and thus the injection needle 1512, are held in place primarily due to the weight of the working fluid in the balloon 1562.

In further embodiments, the injection needle 1512 and stabilizer 1560 are held in place via magnetism. For example, the injection needle 1512 may comprise a magnetic material. Utilization of a magnetic material for the injection needle 1512, in combination with one or more electromagnetic coils or magnets 1550 disposed in desired positions around the patient's head during a subretinal procedure, may enable improved stability of the injection needle 1512 and stabilizer 1560. For example, the one or more electromagnetic coils or magnets 1550 may create a one-dimensional magnetic field for applying a downward force upon the magnetic injection needle 1512 toward the retina, separate from any gravitational force on the injection needle 1512.

Where an electromagnetic coil 1550 is utilized, the electromagnetic coil 1550 may comprise any suitable electromagnetic coil configured to generate a magnetic field upon application of an electric current through the coil. Typically, a direction of a generated magnetic field will be perpendicular to the circular surface of the coil, and can be inverted by changing a direction of the electric current through the coil. To modify a strength or intensity of the generated magnetic field, the electric current applied to the electromagnetic coil 1550 can be increased or decreased. The number and position of electromagnetic coils 1550 may vary depending on the desired positioning of the injection needle 1512 in the patient's eye. In certain embodiments, an electromagnetic coil 1550 may be integrated into a patient head support, a patient table, or any suitable device disposed behind a patient's head during a subretinal injection procedure. As described above, as an alternative to an electromagnetic coil 1550, a magnet 1550 may instead be used.

In still further embodiments, the injection needle 1512 and stabilizer 1560 are held in place via negative pressure. For example, the connector piece 1518 may, in certain embodiments, comprise a port 1552 at a distal end thereof. In such embodiments, the port 1552 is fluidly coupled, via at least one lumen of the tubing 1520, to a vacuum source at the proximal end of the tubing 1520 for generating a negative pressure, or vacuum suction, through the port 1552. Thus, after the injection needle 1512 is positioned at a target injection site on the retinal surface for subretinal injection, the vacuum source may be activated to create vacuum suction through the port 1552 that acts on the retinal surface, thereby immobilizing the injection needle 1512 and stabilizer 1560 against the retina. Generally, the negative pressure generated by the vacuum source is small enough such that it does not cause any damage to the retina, but sufficiently large enough for stabilizing the injection needle 1512 against the retina.

FIG. 15D is a schematic view of the delivery system 1500 of FIG. 15A illustrating an exemplary subretinal delivery device 1501 combined therewith, according to embodiments herein. Meanwhile, FIG. 15E is an enlarged side sectional view of a portion of FIG. 15D illustrating the injection needle 1512 used in connection with the delivery system 1500 described herein. FIGS. 15D-15E are, therefore, described together herein for clarity.

In general, the delivery device 1501 is substantially similar to the other subretinal delivery devices herein, but for the device 1501 being configured to be releasably coupled to the injection needle 1512 and tubing 1520 after the injection needle 1512 is inserted into the subretinal space. The delivery device 1501 includes a tubular injection cannula 1510 which directly engages the injection needle 1512 and which is insertable into the eye. The injection cannula 1510 has an interior channel extending longitudinally from a proximal end 1516 to a distal end 1514 thereof for surrounding the tubing 1520.

The injection cannula 1510 extends from a handpiece 1502 configured to be gripped and handled by the surgeon or surgical assistant. The handpiece 1502 comprises an injection needle release toggle 1540 for releasing the injection needle 1512 from the injection cannula 1510 when the injection needle 1512 is properly positioned and immobilized within the eye. It is contemplated that the release toggle 1540 may comprise any suitable type of mechanical mechanism for releasing the injection needle 1512. For example, in certain embodiments, the release toggle 1540 may comprise a slide button or switch which moves a release mechanism to disengage the injection cannula 1510 from the connector piece 1518, thereby allowing the injection cannula 1510 to be retracted away from the injection needle 1512.

In certain embodiments, the injection cannula 1510 has a slit extending from the proximal end 1516 to the distal end 1514 thereof, thus forming a U-shape in top cross-section. In such embodiments, the delivery device 1501 is configured to be decoupled from the tubing 1520 outside the eye by sliding the tubing 1520 through the slit.

In FIGS. 15D and 15E, the delivery device 1501 is shown in a configuration ready to start a subretinal injection procedure. For example, the delivery device 1501 is coupled to the injection needle 1512, and the injection cannula 1510 of the delivery device 1501 is surrounding the tubing 1520. Furthermore, the stabilizer 1560 is in the retracted position being disposed inside the port 1519c of the connector piece 1518, which is disposed within the distal end 1514 of the injection cannula 1510. In certain embodiments, as shown in FIG. 15E, the injection cannula 1510 of the delivery device 1501 extends beyond the distal end 1522 of the tubing 1520 and surrounds the connector piece 1518 of the injection needle 1512. In some embodiments, the injection cannula 1510 has an inner diameter corresponding to an outer diameter of the connector piece 1518. In some embodiments, the inner diameter of the injection cannula 1510 is between about 0.35 mm and about 0.65 mm, such as between about 0.45 mm and about 0.55 mm, while an outer diameter of the connector piece 1518 is between about 0.4 mm and about 0.7 mm, such as between about 0.5 mm and about 0.6 mm. Other dimensions, however, are also contemplated.

FIG. 15F illustrates a top isometric view of a portion of the delivery system 1500 with an alternative design for injection needle 1582 and stabilizer 1590. In the embodiment of FIG. 15F, the stabilizer 1590 is disposed external to the needle 1582 in both the retracted and extended positions.

As shown, the injection needle 1582 has a proximal end 1584 and a distal end 1586. In certain embodiments, the injection needle 1582 further includes a connector piece 1588 that extends distally from the proximal end 1584 for a length C less than a total longitudinal length N of the injection needle 1582. In certain embodiments, the connector piece 1588 may facilitate connection of the injection needle 1582 to the single- or multi-lumen tubing 1520 described above. For example, the distal end 1522 of the tubing 1520 may attach to the proximal end 1584 of the injection needle 1582 through the connector piece 1588, while the proximal end 1524 attaches to the fluidic drive system (e.g., through a handle as discussed below).

Similar to the connector piece 1518, the connector piece 1588 has one or more internal ports 1578 corresponding to the one or more lumens of the tubing 1520. In the example of FIG. 15F, two ports 1578a and 1578b are shown, each corresponding to and configured to fluidically couple with the distal ends of the lumens 1528a and 1528b, respectively, of the multi-lumen tubing 1520 in FIG. 15B. The two separate ports 1578a, 1578b of the connector piece 1588 merge together toward the distal end 1586 of the injection needle 1582.

The stabilizer 1590 comprises a plurality of flexible, bendable legs 1592, which, in certain embodiments, may be oriented such that they extend along a major longitudinal axis A of the injection needle 1582 when in an “inactive” position. In FIG. 15F, three legs 1592 are shown in an “active” position, which provide a three-point stabilization mechanism for stabilizing the injection needle 1582 at a target injection site on the retinal surface; however, the utilization of more legs 1592 is contemplated, such as four, five, six, or more legs 1592. Each of the plurality of legs 1592 proximally couples to a movable extension ring 1594 that circumscribes the connector piece 1588, and distally couples to a fixed base 1596 of the injection needle 1582 adjacent the distal end 1586. In certain embodiments, a length of the injection needle 1582 extending distally from the base 1582 is equal or substantially equal to the thickness of a retina to allow for traversal into the subretinal space during injection. The legs 1592 are formed of any suitable flexible materials to facilitate bending thereof, including flexible metals such as nitinol and other metallic alloys, as well as flexible thermoplastic polymeric materials such as polyimide.

Again, the stabilizer 1590 is shown in the active position where the legs 1592 of the stabilizer 1590 are bent and a central portion 1583 of each of the legs 1592 extends laterally outward from the connector piece 1588. As described above, in certain embodiments, the legs 1592 may extend substantially parallel with a major longitudinal axis A of the injection needle 1582 when in an inactive position. To transition the stabilizer 1590 to the active position from this inactive position, the extension ring 1594, which may be longitudinally movable along the length C of the connector piece 1588, is actuated in a distal direction 1554 (toward the distal end 1586 of the injection needle 1582). The distal movement of the extension ring 1594 causes the legs 1592 to buckle, or bend, laterally outwardly from the connector piece 1588, since the connector piece 1588 prevents inward bending thereof. As a result, a suitable three-point support is created for stabilizing the injection needle 1582 during injection. To transition the stabilizer 1590 back to the inactive position, the extension ring 1594 may be actuated in a proximal direction 1556, causing the legs 1592 to longitudinally extend, and thus, straighten. In certain embodiments, the extension ring 1594 is lockable in these inactive and active positions, and/or in one or more incremental positions therebetween.

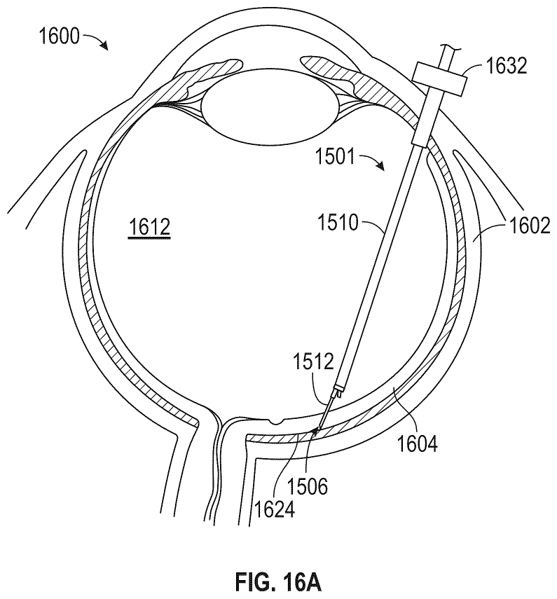

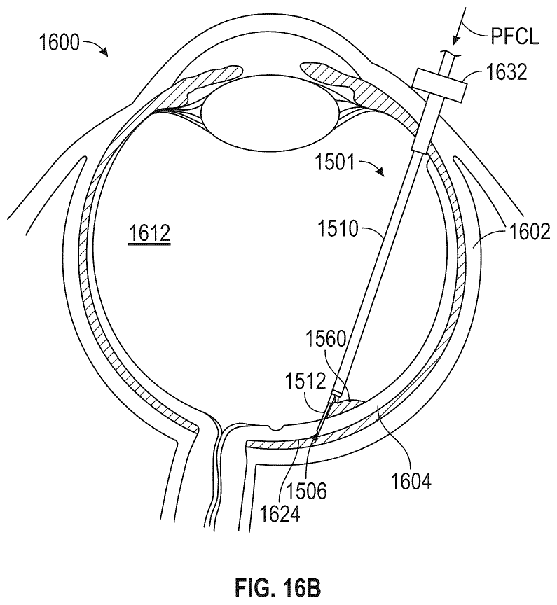

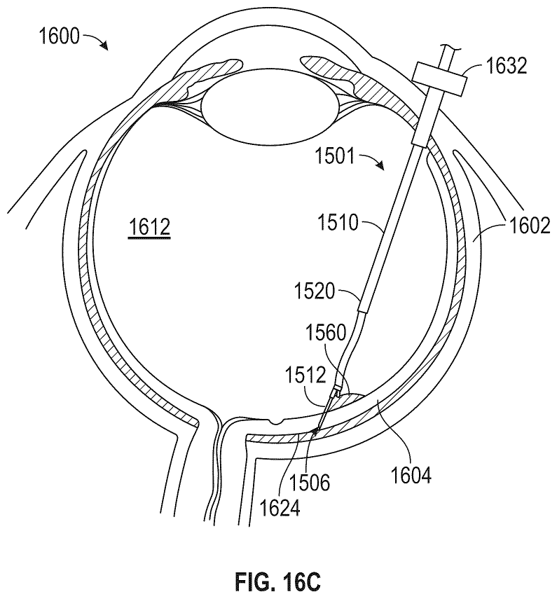

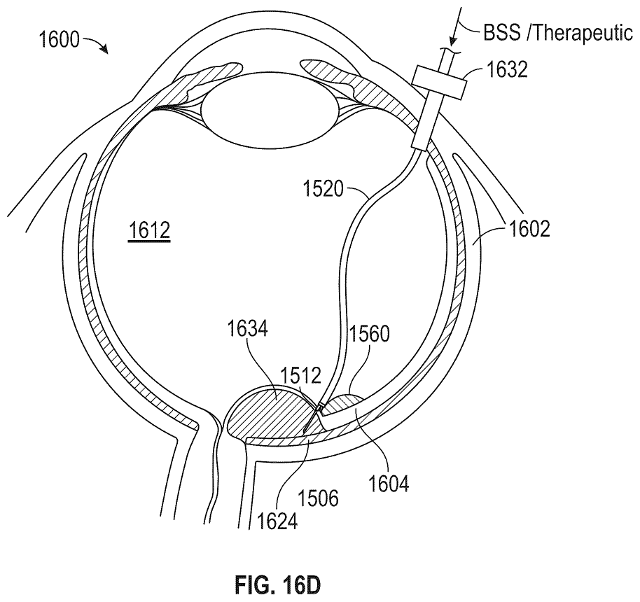

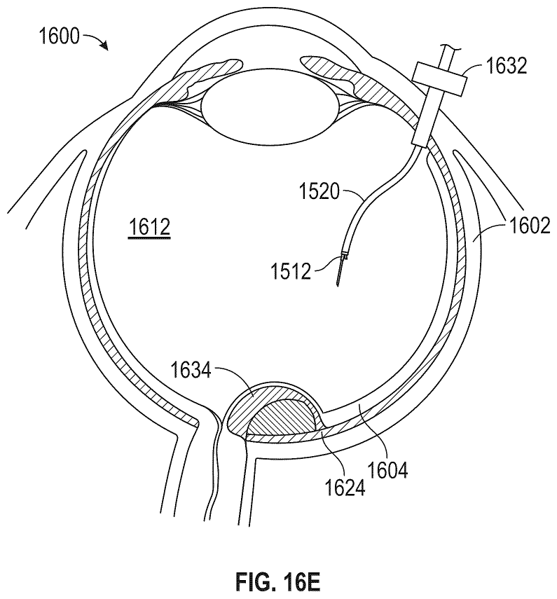

Generally, to actuate the extension ring 1594 in the distal direction 1554 and transition the legs 1592 from the inactive position to the active position, a pushrod or other suitable mechanism on a delivery device injection cannula may be used. For example, prior to decoupling the injection needle 1582 from an injection cannula of a delivery device (as discussed with reference to FIG. 16C below), a user may actuate a toggle on the delivery device handle to distally move a pushrod or other feature on the injection cannula, which may in turn act against the extension ring 1594 and cause the extension ring 1594 to translate distally. The pushrod may interface with the extension ring 1594 via any suitable means, such as hook or clip. In some embodiments, the pushrod may detachably interface with the extension ring 1594.

In some embodiments, to fix the extension ring 1594 in the active position, the extension ring 1594 may be rotated in a first rotational direction over a pin or other locking mechanism. In such embodiments, to facilitate transition of the extension ring 1594 back to the inactive position, the extension ring 1594 may be rotated in a second rotational direction, opposite the first rotational direction, to unlock the extension ring 1594 from the pin or other locking mechanism. In some embodiments, an outer diameter of the connector piece 1588 may gradually increase in the distal direction, thus enabling the extension ring 1594 to be fixed against the connector piece 1588 via mechanical friction. In some embodiments, when transitioning from the active position to the inactive position, the elasticity of the legs 1592 enables the legs 1592, and thus the extension ring 1594, to “spring” back to the elongated inactive position.

In certain embodiments, the extension ring 1594 is perpetually immobilized in a longitudinal position along the connector piece 1588 wherein the legs 1592 are constantly buckled or bent, but may be configured to rotate about the connector piece 1588 to decrease or increase a width of the stabilizer 1590, thereby transitioning the stabilizer from an inactive or active position, respectively. In such embodiments, to transition the stabilizer 1590 to an inactive position, the extension ring 1594 may be rotated in a first rotational direction 1546 to cause the legs 1592 to coil around the connector piece 1588. To transition the stabilizer 1590 to the active position, the extension ring 1594 may be rotated in a second rotational direction 1548 opposite the first rotation direction 1546. Such rotation of the extension ring 1594 may be accomplished via any suitable rotation mechanism on, for example, an injection cannula of a delivery device. In such embodiments, the extension ring 1594 may be rotated to cause the legs 1592 to transition into the active position prior to release of the injection needle 1582 from the injection cannula 1510 for performing subretinal injection, and then again rotated to cause the legs 1592 to transition into the inactive position after the injection cannula 1510 is re-attached to the injection needle 1582 after performing subretinal injection, thus enabling removal from the eye. In some embodiments, however, the injection needle 1582 may be retracted after injection without re-attachment of the injection cannula 1510 thereto, and the legs 1592 may spring back, due their elasticity, to the inactive position upon contact with, e.g., a trocar cannula or other entry cannula when being removed from the eye.

In still other embodiments, rather than utilizing a pushrod or other mechanical feature to actuate the extension ring 1594 and bend the legs 1592, pressured fluids may be utilized to fill, or inflate, the legs 1592, thus causing them to extend laterally from the connector piece 1588. Such pressured fluids may be contained within one or more flexible membranes disposed between the legs 1592.

In further embodiments, the injection needle 1582 and stabilizer 1590 may also be held in place via magnetism and/or negative pressure, as described above with reference to injection needle 1582. For example, in certain embodiments, the injection needle 1582 may comprise a magnetic material configured to be acted upon by a magnetic force applied thereto. In certain embodiments, the injection needle 1582 may comprise a port through a distal surface of the base 1596, which may be fluidly coupled to, via at least one lumen of the tubing 1520, a vacuum source at the proximal end of the tubing 1520 for generating a negative pressure, or vacuum suction, through the port.

FIGS. 16A-16E illustrate transverse sectional views of an eye 1600 at different steps of performing a subretinal injection with the delivery system of FIGS. 15A-15E having the stabilizer 1560, according to certain embodiments. Although described and illustrated with the stabilizer 1560, the operations in FIGS. 16A-16E may also be performed with other stabilizing mechanisms, including the stabilizer 1580 in FIG. 15F.

Turning now to FIG. 16A, in preparation for the subretinal injection, sclera 1602 is incised using a trocar cannula which consists of a valved insertion cannula 1632 and a trocar, as described above with reference to FIG. 2. The trocar is removed from the eye 1600, leaving the valved insertion cannula 1632 in place. Then, the injection cannula 1510 of the delivery device 1501 is inserted into the eye 1600 through the valved insertion cannula 1632, and the distal end 1506 of the injection needle 1512 is guided through the vitreous chamber 1612 and inserted into the subretinal space 1624 at a target injection site on the surface of the retina 1604.

Thereafter, at FIG. 16B, the injection needle 1512 is immobilized at the target injection site on the surface of the retina 1604 using the stabilizer 1560. In certain embodiments, a pressure or fluid is applied through a lumen (e.g., lumen 1528c) of the tubing 1520 to extend the stabilizer 1560 from the connector piece 1518 to place the stabilizer 1560 in contact with the surface of the retina 1604. The stabilizer 1560 is configured to securely contact the retina 1604 in such a way that the injection needle 1512 is immobilized at the target injection site on the surface of the retina 1604. In certain embodiments, the stabilizer 1560 is formed from a material that is conformal to the surface of the retina 1604 to increase the contact area therebetween.

In embodiments where magnetism is used to stabilize the injection needle 1512, a magnetic field acting may be provided (e.g., via coils or magnets) to act upon the injection needle 1512 and immobilize it. In embodiments where negative pressure is used to stabilize the injection needle 1512, a vacuum source may be activated to supply vacuum suction at, e.g., port 1552.

At FIG. 16C, after the stabilizer 1560 is in contact with the surface of the retina 1604, the injection cannula 1510 of the delivery device 1501 is retracted from the eye 1600. Upon retraction of the injection cannula 1510, the injection needle 1512 and the tubing 1520 are decoupled by external forces. As used herein, external forces generally include any forces applied to the injection needle 1512 or the tubing 1520 from outside the eye 1600. For example, external forces generally include light and/or inadvertent movement of any part of the delivery system 1500 or delivery device 1501 by the surgeon or surgical assistant. In certain embodiments, the decoupling limits the effect of external forces associated with injection and/or external forces associated with movement of handheld instruments such as the delivery device 1501. In certain embodiments, an excess length of the tubing 1520 is provided in an unconstrained state inside the eye 1600 to facilitate decoupling. It will be appreciated that when external forces are applied to the tubing 1520, the excess length enables movement of the tubing 1520 inside the eye 1600 without transfer of force to the injection needle 1512.

At FIG. 16D, injection fluids (e.g., a non-treatment and/or a treatment solution) are injected from the fluidic drive system 1570 to the subretinal space 1624 via the one or more lumens of the tubing 1520.

In certain embodiments, the injection fluids are injected in a one-step procedure, which may greatly simplify fluid handling of the injection fluids and the overall injection procedure. For example, the fluidic drive system 1570 may comprise a fluid reservoir 1574 storing a mixture of both treatment solution and non-treatment solution, herein referred to as a “premixed” solution. The fluid pump 1572 may thus drive flow of the premixed solution through a singular lumen of the tubing 1520 to inject the premixed solution to the subretinal space 1624 in a single step. Because the treatment solution and non-treatment solution are premixed, a precise dosage of a therapeutic substance may be delivered using this one-step approach.

As shown in FIG. 16D, in certain embodiments, the injection of the premixed solution forms a bleb 1634 in the subretinal space 1624 between the retina 1604 and the retinal pigment epithelium (RPE) 1630, which is a localized hemispherical lifting of the retina 1604. Because the injected fluid in the one-step procedure comprises premixed treatment and non-treatment solutions, the dispersion of the injected fluid within the bleb 1634 may be more homogenous.

In certain other embodiments, the injection fluids are injected in a two-step procedure, in which a non-treatment solution and treatment solution are injected separately without premixing. For example, the non-treatment solution may first be injected from the fluidic drive system 1570 to the subretinal space 1624 via one of the lumens (e.g., lumen 1528b) of a multi-lumen tubing 1520. This first step may form the initial bleb 1634 in the subretinal space 1624. Thereafter, the treatment solution may then be injected from the fluidic drive system 1570 to the subretinal space 1624 via another one of the lumens (e.g., lumen 1528a) of a multi-lumen tubing 1520, thereby expanding the bleb 1634 from its initial size. The utilization of this two-step procedure may be particularly beneficial for clinical studies wherein an ideal or preferred concentration of a therapeutic substance has not yet been determined, as the therapeutic substance may be gradually and separately injected form the non-treatment solution, thereby providing higher dosage flexibility.

In certain embodiments, injecting the premixed solution, or each of the non-treatment solution and the treatment solution, is performed hands-free. For example, the fluid pump 1572 may drive flow of each of the premixed solution, non-treatment solution, treatment solution, and/or working fluid without manual actuation of the plurality fluid reservoirs 1574. In certain embodiments, the fluid pump 1572 operates according to instructions received from the controller 1576. In certain embodiments, the controller 1576 receives control signals via a wireless receiver. In certain embodiments, the surgeon or surgical assistant may control pressure or volume of injection of each of the fluids using a foot pedal (e.g., foot pedal 410) which is in wireless communication with the controller 1576 via the wireless receiver and/or an antenna.

At FIG. 16E, after completing the injection of the injection fluids, the injection needle 1512 may be remobilized by retracting the stabilizer 1560 into the connector piece 1518, thereby removing the stabilizer 1560 from being in contact with the surface of the retina 1604. In certain embodiments, working fluid is removed from the stabilizer 1560 and/or corresponding lumen using vacuum pressure to cause the stabilizer 1560 to retract therein. In some embodiments, the stabilizer 1560 is removed from being in contact with the retina 1604 without being retracted into the connector piece 1518. In embodiments where magnetism is used to stabilize the injection needle 1512, the magnetic field acting upon the injection needle 1512 may be suppressed or inactivated. In embodiments where negative pressure is used to stabilize the injection needle 1512, the vacuum source supplying the negative pressure at, e.g., port 1552 may be inactivated.

Thereafter, the tubing 1520 and the injection needle 1512 coupled thereto may be removed from the eye 1600. In certain embodiments, the injection site may remain unpatched. In some other embodiments, the injection site may be filled with a sealing agent (e.g., fibrin glue, collagen, cyanoacrylate, cellular attachment factors, fibronectin, laminin, extracellular matrix-based hydrogels, polyacrylic acid, zinc polycarboxylate cement, silicone adhesive, or an ophthalmic viscosurgical device (OVD), or viscoelastic plug).

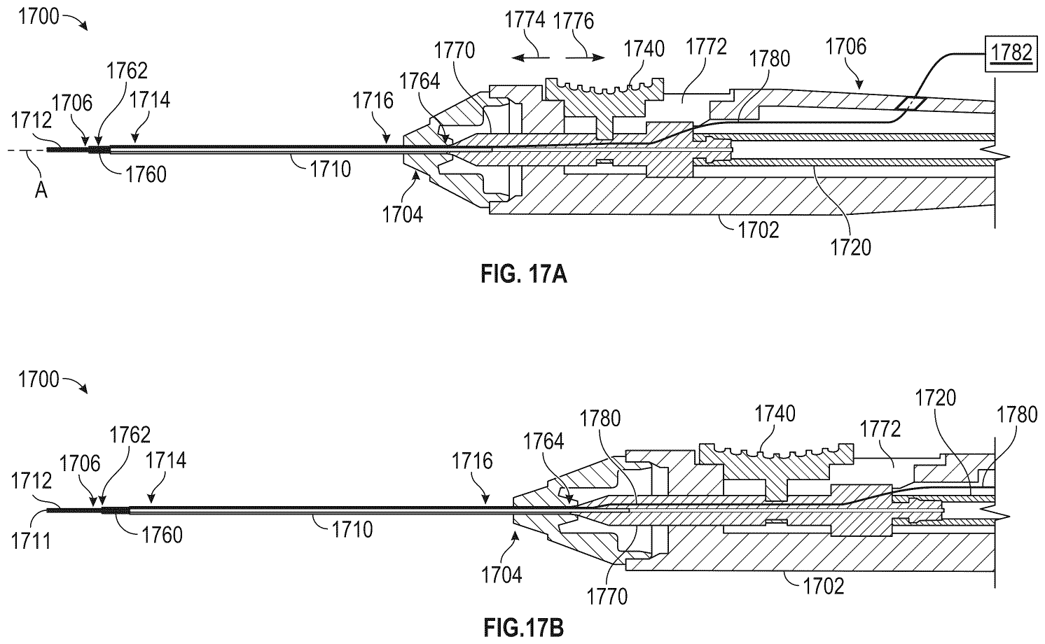

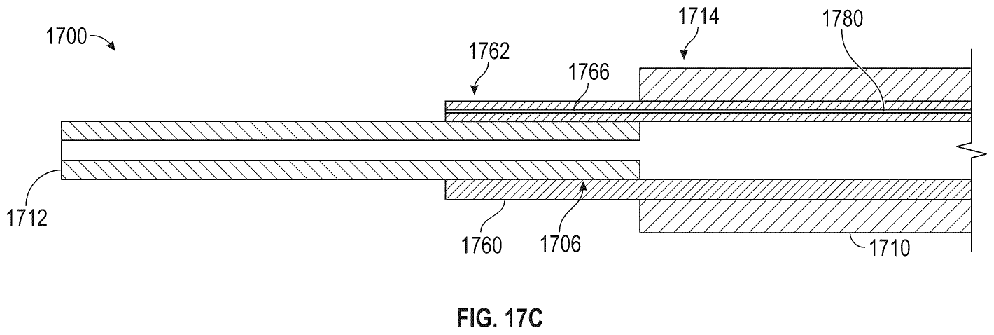

FIGS. 17A-17C illustrate cross-sectional side views of an exemplary subretinal delivery device 1700 configured to be used in conjunction with an optical coherence tomography (OCT) system to provide OCT guidance during subretinal injection procedures, according to certain embodiments of the present disclosure. The delivery device 1700 may be used as, e.g., the delivery device 414 of the surgical system 400 of FIG. 4, or other surgical systems for subretinal injection as described herein. Further, aspects of the delivery device 1700 may be combined with other delivery devices and/or components described herein without limitation.

Subretinal injections are generally very delicate procedures since they require the puncturing of one or more tissues/membranes of the eye to access the subretinal space, and thus, such procedures require a great amount of skill by the surgeon to minimize trauma. To assist surgeons during such procedures and improve the safety thereof, OCT-based guidance may be used. OCT is an imaging technique that uses low-coherence light to capture micrometer-resolution, one- and two- and three-dimensional (e.g., cross-sectional) images from within a biological tissue in real-time. During an ophthalmic procedure such as a subretinal injection, OCT may be utilized to: determine a general structure of an ocular tissue or layer; measure a distance from a probe tip to an ocular tissue or layer; and/or measure a thickness of an ocular tissue or layer, among other things. Thus, when utilized during a subretinal injection, OCT may assist the surgeon in accurately placing a delivery device injection cannula and/or injection needle within the eye for injection.

Turning now to FIGS. 17A and 17B, delivery device 1700 includes a handle 1702 and a tubular injection cannula 1710, wherein a proximal end 1716 of the injection cannula 1710 is coupled to and extends distally from a distal end 1704 of the handle 1702. Extending from a distal end 1714 of the injection cannula 1710 is an inner fluidic shaft 1760, and within the inner fluidic shaft 1760 is a curved or substantial straight injection needle 1712 (a straight injection needle 1712 is shown). The injection needle 1712 is fixedly coupled to and extends distally from a distal end 1762 of an inner fluidic shaft 1760, which may have a larger diameter than that of the injection needle 1712. Thus, in such embodiments, the distal end 1762 of the inner fluidic shaft 1760 may circumscribe the injection needle 1712 for a given length of a proximal end 1706 of the injection needle 1712.

The inner fluidic shaft 1760 is configured to slidably extend from and retract into the distal end 1714 of the injection cannula 1710 by actuation of a toggle 1740 on the handle 1702, which may be a sliding toggle. In the embodiments of FIGS. 17A and 17B, a proximal end 1764 of the inner fluidic shaft 1760 fluidically couples to a slider 1770 (or base of the toggle 1740), which is disposed through an inner cavity 1772 of the handle 1702 and connects to the toggle 1740. Actuation (here, sliding) of the toggle 1740 causes translation of the slider 1770 within the handle 1702 and along a major longitudinal axis A of the handle 1702 and injection cannula 1710. Accordingly, translation of the toggle 1740 in a first, distal direction (shown as arrow 1774) may cause the slider 1770 to translate distally within the cavity 1772, thereby causing the inner fluidic shaft 1760 and injection needle 1712 coupled thereto to extend from the injection cannula 1710. In a fully extended position, at least a portion of both the inner fluidic shaft 1760 and the injection needle 1712 are exposed from the injection cannula 1710. Meanwhile, translation of the toggle 1740 in a second, proximal direction (shown as arrow 1776) may cause the slider 1770 to translate proximally within the cavity 1772, thereby causing the inner fluidic shaft 1760 and injection needle 1712 coupled thereto to retract into the injection cannula 1710. Note that the actuation mechanism in FIGS. 17A and 17B is only exemplary, and that other actuation mechanisms for translating the slider 1770 are also contemplated, such as a deformable basket, button, or the like.

A flexible fluidic tubing 1720 for supplying injection fluids (e.g., non-treatment and/or a treatment solutions) to the delivery device 1700 is disposed through a proximal end 1706 of the handle 1702 and fluidically coupled to the slider 1770 within the handle 1702. The flexible fluidic tubing 1720, slider 1770, inner fluidic shaft 1760, and injection needle 1712 form a single, continuous channel for flowing fluids during a subretinal injection.

Again, the delivery device 1700 of FIGS. 17A-17C is configured to be used in conjunction with an OCT system to provide OCT-based guidance during the performance of a subretinal injection procedure. To enable OCT imaging during the subretinal delivery of fluids, an optical fiber 1780 is disposed through the proximal end 1706 of the handle 1702, through the cavity 1772 and the injection cannula 1710, and distally terminates at the inner fluidic shaft 1760 (see FIG. 17C for magnified view). In certain embodiments, such as the examples of FIGS. 17A and 17B, the optical fiber 1780 may extend through the slider 1770 or base of the toggle within the cavity 1772.

The optical fiber 1780 is coupled to an OCT system 1782, which may include any suitable type of OCT device, such as a time or frequency domain OCT device, a Fourier OCT device, etc., to provide short-range or long-range one-dimensional (e.g., from a central point), two-dimensional, and/or three-dimensional images of anatomical structures within the patient's eye in real-time. Such OCT imaging may then be utilized to determine measurements of various individual or collective physical parameters of the patient's eye, including the shape and thickness of various membranes. In addition, the OCT imaging may be utilized to determine a distance and/or position of the distal tip 1711 of injection needle 1712, or distal ends 1762 and 1714 of the inner fluidic shaft 1760 and injection cannula 1710, respectively, in relation to ocular tissues (such as the retina) during performance of ophthalmic procedures. Accordingly, visualization with the OCT system 1782 may be used by a surgeon during performance of a subretinal injection procedure to guide the placement of the injection needle 1712, such as between the sensory retina and the RPE, without causing any unnecessary damage to surrounded tissues, thereby improving the safety and ease of such procedures.

Turning now to FIG. 17C, the injection needle 1712 and the distal ends 1762 and 1714 of the inner fluidic shaft 1760 and injection cannula 1710, respectively, are shown. In this example, the optical fiber 1780 is disposed through a bore 1766 in the cylindrical wall of the inner fluidic shaft 1760 and terminates at the distal end 1762 thereof. In certain other embodiments, the optical fiber 1780 may be fixedly attached to, e.g., a groove in an outer surface of the wall of the inner fluidic shaft 1760, such as by an adhesive, and may terminate the any point along the length of the inner fluidic shaft 1760. Because the inner fluidic shaft 1760 is fixedly attached to the injection needle 1712 and translates therewith, the distance between the distal terminal end of the optical fiber 1780 and the distal tip 1711 of the injection needle 1712 remains constant during use, thereby enabling continuous, accurate OCT measurements of the distance between the distal tip 1711 and ocular tissues during performance of subretinal injection procedures.

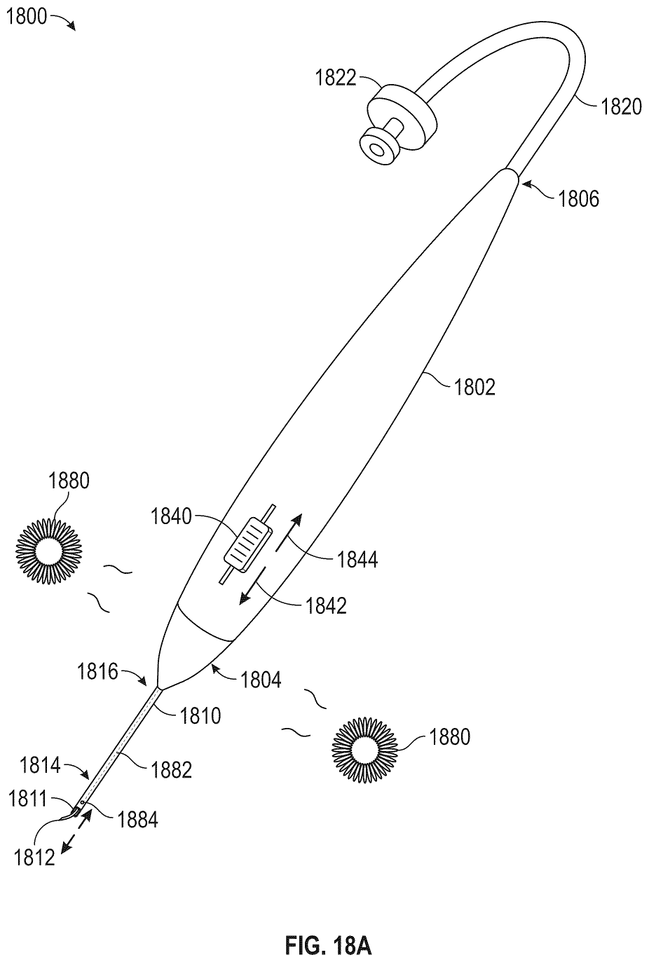

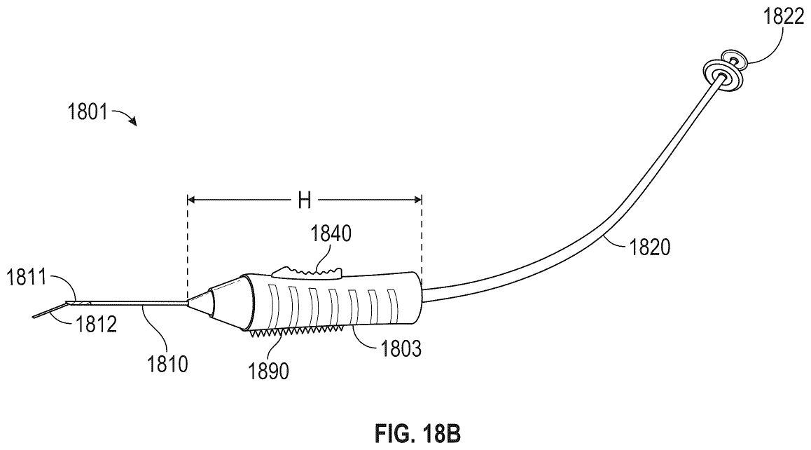

FIGS. 18A and 18B illustrate perspective views of exemplary subretinal delivery devices 1800 and 1801, respectively, according to certain embodiments of the present disclosure. The delivery devices 1800 and 1801 may be used as, e.g., the delivery device 414 of the surgical system 400 of FIG. 4, and aspects thereof may be combined with other delivery devices and/or components described herein without limitation. Certain aspects of the delivery devices 1800 and 1801 are particularly beneficial for performing subretinal injections (and other related procedures) via a suprachoroidal approach, as described above with reference to FIG. 3.

Turning now to FIG. 18A, the delivery device 1800 includes a handle 1802 and a tubular injection cannula 1810 having a proximal end 1816 coupled to and extending distally from a distal end 1804 of the handle 1802. A distal end 1814 of the injection cannula 1810 comprises a distal tip 1811, which may in certain embodiments be tapered or sloped relative to a major longitudinal axis of the injection cannula 1810 in order to facilitate separation of the choroid from the sclera as the injection cannula 1810 is moved through the suprachoroidal space. In certain embodiments, the distal tip 1811 may have oval-like, widened, or flattened cross-section, to facilitate easier translation through the suprachoroidal space. Exemplary distal tips are discussed below in more detail. The injection cannula 1810 and/or distal tip 1811 are generally formed of any suitable flexible surgical-grade materials, such a metallic or thermoplastic polymeric materials. Examples of flexible metallic materials include nitinol and other metallic alloys. Examples of suitable thermoplastic polymeric materials include polyimide. In certain embodiments, the distal tip 1811 is formed of a rigid material, and the remainder of the injection cannula 1810 is formed of a flexible material.

In certain embodiments, the distal tip 1811 may comprise a magnetic material. Utilization of a magnetic material for the distal tip 1811, in combination with one or more electromagnetic coils 1880 disposed in desired positions around the patient's eye, may enable improved maneuverability of the injection cannula 1810 and distal tip 1811 in the suprachoroidal space for subretinal injection. For example, the one or more electromagnetic coils 1880 may be activated to create a one-dimensional, two-dimensional, or three-dimensional magnetic field and apply force upon the magnetic distal tip 1811 to steer the distal tip 1811 within the suprachoroidal space, separate from any force applied on the delivery device 1800 by a user to move the distal tip 1811 “forward” through the suprachoroidal space (e.g., away from the entry point into the eye, or “sclerotomy”). This magnetic steering of the distal tip 1811 facilitates easier and more precise handling and maneuverability of the distal tip 1811 and injection cannula 1810 while the distal tip 1811 and injection cannula 1810 are moved and/or positioned in the suprachoroidal space for subretinal injection. In certain aspects, the magnetic steering also enables improved ergonomics for the surgeon during an insertion procedure, as the physical burden on the surgeon for proper placement and maneuvering of the distal tip 1811 within the suprachoroidal space is reduced.

In embodiments where one or more electromagnetic coils 1880 are utilized to create a one-dimensional magnetic field, a one-dimensional force can be applied to the magnetic distal tip 1811 to steer the distal tip 1811 in a first lateral direction and a second lateral direction opposite the first direction, separate from any forces pushing the distal tip 1811 forward. In such embodiments, the first lateral direction and second lateral direction are each perpendicular to a major longitudinal axis A of the injection cannula 1810 that disposed along a longitudinal length thereof, and are also tangential to the suprachoroidal space. Further, the one-dimensional force may not be strong or intense enough to push or pull the distal tip 1811 on its own; rather, it may be limited such that it only assists in steering the distal tip 1811, and any actual movement of the distal tip 1811 is caused by manual application of force on the handle 1802 by the user.

In embodiments where two or more electromagnetic coils 1880 are utilized to create a two-dimensional magnetic field or three-dimensional magnetic field, a two-dimensional force or three-dimensional force can be applied to the magnetic distal tip 1811 to steer the distal tip 1811 in one or more directions additional to the first lateral direction and second lateral direction. In such embodiments, the magnetic distal tip 1811 (and thus, the injection cannula 1810) may be moved through the suprachoroidal space without any forces applied to the handle 1802 by the user-rather, the distal tip 1811 and injection cannula 1810 may be entirely controlled and positioned by application and modification of a two-dimensional magnetic field or three-dimensional magnetic field acting upon the distal tip 1811.

Generally, the electromagnetic coils 1880 may comprise any suitable electromagnetic coils configured to generate a magnetic field upon application of an electric current through the coils. Typically, a direction of a generated magnetic field will be perpendicular to the circular surface of an electromagnetic coil, and can be inverted by changing a direction of the electric current through the coil. To modify a strength or intensity of a generated magnetic field, the electric current applied to the electromagnetic coils 1880 can be increased or decreased. The number and position of electromagnetic coils 1880 may vary depending on the desired insertion direction for the distal tip 1811 and the positioning of the patient's eye. In certain embodiments, the electromagnetic coils 1880 may be integrated into a patient head support and/or an operating table or surgical bed. For example, where integrated into a head support, one coil may be placed in the head support above the patient's head, one coil may be placed in the head support behind the patient's head, and another coil may be placed in the head support on either lateral side of the patient's head.

In certain embodiments, at least a portion of a distal end 1814 of the injection cannula 1810, such as the distal tip 1811, comprises a photoluminescent material, such as a phosphorescent material. For example, in certain embodiments, the distal tip 1811 of the injection cannula 1810 may comprise a material comprising phosphors, thereby radiating visible light after being energized by, e.g., visible light. The utilization of a photoluminescent material for the distal tip 1811 (or other portion of the distal end 1814) enables visibility of the position thereof, through the choroid and retina, as the injection cannula 1810 is moved through the suprachoroidal space, via a microscope or other viewing system having a point-of-view directed at the retina through the lens or sclera. For example, prior to insertion of the injection cannula 1810 into the patient's eye during a procedure, the distal tip 1811 may be exposed to a visible light source for a suitable amount of time to energize the photoluminescent material. Thereafter, upon insertion and movement of the injection cannula 1810 through the suprachoroidal space, the distal tip 1811 will continuously emit light, which can be seen through the choroid and retina by the microscope or other viewing system. Such visibility of the position of the distal tip 1811 or other portion of the distal end 1814 facilitates efficient positioning of the injection cannula 1810 for subretinal injection at a target injection site.

In certain embodiments, the injection cannula 1810 comprises a light fiber 1882 (dashed line) having a distal end 1884 terminating at or near the distal tip 1811 and configured to emit light therefrom. The light fiber 1882 may extend proximally through the injection cannula 1810, through the handle 1802, and be optically coupled to any suitable visible light source, such as a white light source, internal or external to the handle. For example, the light fiber 1882 may be optically coupled to a light source integrated with a surgical console. During a subretinal injection procedure, while the injection cannula 1810 is inserted into and moved through the suprachoroidal space, the light source may be activated by the surgeon to emit visible light from the distal end 1884 of the light fiber 1882, which can be seen through the choroid and retina using a microscope or other viewing system having a point-of-view directed at the retina through the lens or sclera. Accordingly, the light emitted from the light fiber 1882 may be utilized to guide positioning of the distal end 1814 of the injection cannula 1810 for efficient and accurate placement thereof near a target injection site during subretinal injection procedures. In certain embodiments, the light fiber 1882 comprises a single core optical fiber; in certain embodiments, the light fiber 1882 comprises a multi-core optical fiber. Generally, one or more claddings may circumscribe or surround the one or more cores of the light fiber 1882.

As further shown in FIG. 18A, a curved or straight injection needle 1812 (a curved needle is shown) is disposed within the injection cannula 1810 for piercing desired ocular tissues (here, the choroid and RPE) at an angle relative to a major longitudinal axis of the injection cannula 1810 to deliver a fluid to the subretinal space. In exemplary embodiments, the injection cannula 1810 is a 23-, 25-, or 27-gauge needle, while the injection needle 1812 is a finer gauge needle, such as a 38-gauge needle. However, other sizes/gauges of injection cannulas and injection needles may be used in other embodiments. In certain embodiments, the injection needle 1812 is formed of a similar material to the injection cannula 1810 and/or the distal tip 1811.

In certain embodiments, the injection needle 1812 is configured to slidably extend from and retract into a distal end 1814 of the injection cannula 1810, which facilitates the prevention of damage to the injection needle 1812 during insertion and/or movement of the injection cannula 1810 in an eye. Such actuation of the injection needle 1812 may be controlled by any suitable mechanism. In the example of FIG. 18A, actuation of the injection needle 1812 is controlled by a toggle 1840 of the handle 1802. In certain embodiments, the toggle 1840 comprises a sliding button or switch, wherein sliding of the toggle 1840 by a user (e.g., a surgeon) in a distal direction 1842 causes the injection needle 1812 to extend from the injection cannula 1810, and sliding of the toggle 1840 in a proximal direction 1844 causes the injection needle 1812 to retract into the injection cannula 1810.

In certain embodiments, a sliding toggle 1840 may also be lockable, such that the injection needle 1812 may be fixed in either an extended or a retracted position. Locking of the injection needle 1812 prevents unintended movement of the injection needle 1812 during a retinal procedure, e.g., a subretinal injection, thereby reducing the risk of unwanted tissue damage and improving the overall safety of such procedures. In one example, to unlock/release the sliding toggle 1840 for adjustment, the toggle 1840 may be continuously depressed by a user, allowing the user to freely slide the toggle 1840 and thus, freely extend or retract the injection needle 1812. In this example, the toggle 1840 may only be movable while depressed (e.g., activated) by the user. Correspondingly, releasing the toggle 1840 may cause the toggle 1840 to raise and lock in place, thereby locking the injection needle 1812 in place. Such a push button locking mechanism may be facilitated, in part, by a spring lever disposed with the handle 1802, as well as one or more tracks comprising grooves or notches along which the toggle 1840 may slide.

In certain embodiments, the injection needle 1812 is coupled to an inner fluidic shaft at least partially disposed within the cannula 1810 for fluidic coupling between the injection needle 1812 and the toggle 1840 or fluidic tubing. In such embodiments, the inner fluidic shaft may be slidably disposed within the cannula 1810 to facilitate extension and retraction of the injection needle 1812 upon actuation of the toggle 1840.

In certain embodiments, a flexible fluidic tubing 1820 for supplying injection fluids (e.g., non-treatment and/or a treatment solutions) to the delivery device 1800 may be disposed through a proximal end 1806 of the handle 1802 and fluidically coupled to the injection needle 1812 within the handle 1802. In certain embodiments, the fluidic tubing 1820 may couple to the proximal end 1806 of the handle 1802, or another fluidic tubing within the handle 1802 (described elsewhere herein). Generally, the fluidic tubing 1820 comprises a supply line through which non-treatment and/or a treatment solutions from a fluid source may be provided to the delivery device 1800 for delivery to an eye. In certain embodiments, the fluid source comprises a fluidic system, which may be coupled to the fluidic tubing 1820 via connection 1822, such as a Luer lock or other male-female coupling. In certain other embodiments, the handle 1802 may comprise an actuatable internal chamber fluidically coupled to the injection cannula 1810 and containing the injection fluids. In such embodiments, subretinal delivery device 1800 may not be coupled to any external fluidic tubing.

In further embodiments, to simplify fluidic preparation for subretinal injection and/or the injection itself, a therapeutic agent may be provided to the delivery device 1800 from a prefilled cartridge (not shown in FIG. 18A) that can be coupled to a fluidic drive system of the delivery device 1800, or to an external fluidic system connected to the delivery device 1800 via the fluidic tubing 1820. In certain embodiments, the prefilled cartridge comprises a single lumen containing a premixed treatment solution comprising constituents mixed in desired ratios and/or concentrations in appropriate buffer solutions. Such embodiments facilitate one-step subretinal injection procedures, wherein a bleb may be formed with a premixed therapeutic substance instead of first forming the bleb with a buffer solution and then injecting a therapeutic substance into the bleb. Accordingly, utilizing prefilled and premixed cartridges may facilitate more efficient and accurate dosage concentration control. In still other embodiments, a prefilled cartridge may comprise two or more lumens containing unmixed therapeutic substances, which can be automatically or semi-automatically mixed within, e.g., a fluidic system or the delivery device, before performing subretinal injection. Cartridges for therapeutic agents are described in further detail below.

In certain embodiments, the injection needle 1812 may be further fluidically coupled to a second prefilled cartridge or other fluid source configured to supply a colorant or marker fluid to the injection needle 1812. In such embodiments, the second prefilled cartridge or other fluid source may be configured to flow the colorant or marker fluid to the injection needle 1812 as the needle is extended from the injection cannula 1810 and pierces the choroid. Accordingly, in such embodiments, the colorant or marker fluid provides visualization of the position of the injection needle 1812 during injection, and may be utilized to prevent the injection needle 1812 from being extended past the subretinal space and into the sensory retina. The colorant or marker fluid may be viewed by the user via a microscope or other viewing system having a point-of-view directed at the retina through the lens or sclera.

Turning now to FIG. 18B, the delivery device 1801 is substantially similar to the delivery device 1800 but for the handle 1803. In FIG. 18B, the handle 1803 may be described as a “minimal” handle, since the handle 1803 is reduced in size to the absolute minimum, or near absolute minimum, dimensions for extending and retracting the injection needle 1812 from the injection cannula 1810. For example, the handle 1803 has a length H that is the minimum length necessary for facilitating actuation of the toggle 1840, by a user, to fully extend and retract the injection needle 1812. In FIG. 18B, the toggle 1840 comprises a sliding button, and thus, the length H is the minimum length necessary to support the toggle 1840 when translated to a first position wherein the injection needle 1812 is full extended form the cannula 1810, and also a second position wherein the injection needle 1812 is fully retracted into the cannula 1810.

In certain embodiments, the handle 1803 is also formed of a lightweight material. For example, the handle 1803 may be formed of a lightweight thermoplastic polymeric material, which may generally be rigid. In certain examples, the handle 1803 comprises polyether ether ketone (PEEK), polyetherketone (PEK), and/or polytetrafluoroethylene (PTFE).

The reduced dimensions and/or lightweight construction of the handle 1803 allow a surgeon to shift their focus to the orientation and positioning of the injection cannula 1810 and/or distal tip 1811 during entry and traversal of the suprachoroidal space, rather than handling of the handle 1803. For example, during a conventional subretinal injection using a suprachoroidal approach, the surgeon may simultaneously utilize two sets of forceps: one set for holding open an incision in the sclera for entry of a flexible injection cannula of a delivery device, and another set for holding and inserting the injection cannula. If the delivery device comprises a large and/or heavy handle, the delivery device must also be supported during the procedure, thereby complicating the procedure since the surgeon only has two hands. In such situations, another member of the surgical staff may need to hold the delivery device while the surgeon guides the injection cannula into the patient's eye. However, the utilization of a “minimal” handle, such as the handle 1803 in FIG. 18B, circumvents such complications. Because the handle 1803 is small in size and/or light in weight, neither the surgeon nor the surgical assistant need to hold the handle during the performance of a subretinal injection; instead, the handle 1803 may be free-hanging, as its size and weight allow it to do so without disturbing the procedure. Thus, the surgeon may instead focus all of their attention on manipulating the injection cannula 1810.

In further embodiments, the handle 1803 may comprise a velcro strip 1890, or other fastening device, which may be fastened to a corresponding feature on a headband or other article disposed or secured to, e.g., the head or other body part of a patient during the performance of a subretinal injection.

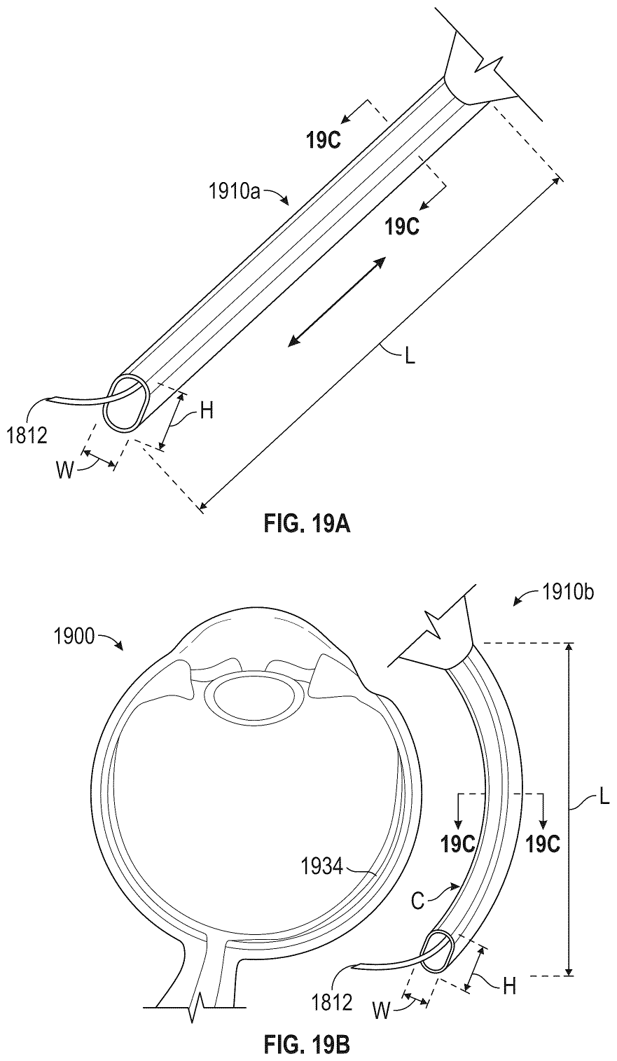

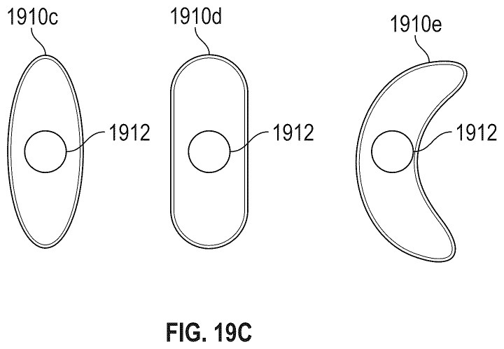

FIGS. 19A-19C illustrate various views of exemplary injection cannulas which may be used with the delivery devices 1800 and 1801 of FIGS. 18A-18B, or other delivery devices for subretinal injection as described herein, according to certain embodiments of the present disclosure. More particularly, FIGS. 19A and 19B illustrate perspective views of straight and curved injection cannulas 1910a and 1910b, respectively, while FIG. 19C illustrates schematic cross-sectional top views of an injection cannula 1910c to demonstrate various exemplary cross-sectional profiles of injection cannulas for use with delivery device 1800.

As shown in FIG. 19A, in certain embodiments, the delivery device 1800 for performing a suprachoroidal subretinal injection comprises a straight or substantially straight injection cannula 1910a. To facilitate easy traversal/sliding of the suprachoroidal space during such procedures, the injection cannula 1910a may be formed of a highly flexible material, which allows the injection cannula 1910a to conform to the curvature of the suprachoroidal space when disposed therein, thereby reducing strain on the choroid caused by the injection cannula 1910a. Accordingly, the flexibility of the shaft may reduce or eliminate any damage caused to the retina and/or choroid during the positioning of the injection cannula 1910a for subretinal delivery of fluids.

In certain embodiments, the injection cannula 1910a is formed of any suitable flexible surgical-grade metallic materials. Examples of flexible metallic materials include nitinol and other metallic alloys. In certain embodiments, the injection cannula 1910a is formed of any suitable flexible thermoplastic polymeric materials. Examples of suitable thermoplastic polymeric materials include polyimide, thermoplastic polyurethane (TPU), polyether block amide (PEBA), and the like.

In certain embodiments, the injection cannula 1910a comprises a lateral width W along a length L of the injection cannula 1910a that is greater than a vertical height H along the length L. Accordingly, in such embodiments, the injection cannula 1910a may be described as being substantially “wide” and/or “flat.” These dimensions of the injection cannula 1910a may be advantageous when accessing and traversing the suprachoroidal space by distributing strain along a wider surface (width W) of the injection cannula 1910a, thereby reducing the expansion of the suprachoroidal space when the injection cannula 1910a is passed therethrough and facilitating reduced choroidal and retinal damage. Even further, the wide and/or flat morphology of the injection cannula 1910a enables better directional control of the injection cannula 1910a by the user by reducing the lateral flexibility/bendability of the injection cannula 1910a in a direction parallel to the width W.

Turning now to FIG. 19B, in certain embodiments, the delivery device 1800 for performing a suprachoroidal subretinal injection comprises a curved injection cannula 1910b. While the injection cannula 1910b may, in certain examples, be substantially similar to the injection cannula 1910a in terms of materials and/or dimensions (e.g., being flexible and substantially wide and/or flat), the injection cannula 1910b comprises a predefined curvature C along the length L as compared to the straight disposition of the injection cannula 1910a. In certain embodiments, the curvature C of the injection cannula 1910b matches or substantially matches a curvature of the eye (eye 1900 is shown for reference) in the suprachoroidal space 1934 so as to reduce strain caused along the suprachoroidal space 1934 when the injection cannula 1910b is passed therethrough, thereby reducing any damage caused to the choroid and/or retina. In certain embodiments, to facilitate the curvature C of the injection cannula 1910b, the injection cannula 1910b may be formed of a stiffer material as compared to those materials recited above with reference to injection cannula 1910a. For example, in certain embodiments, the injection cannula 1910b may comprise aluminum, stainless steel, nitinol, and other metallic alloys. In certain embodiments, the injection cannula 1910b comprises polyimide, polyurethane (PUR), combinations thereof, or the like.

FIG. 19C illustrates schematic cross-sectional top views (along a major longitudinal axis) of injection cannulas 1910c-1910e to demonstrate various exemplary cross-sectional profiles of injection cannulas for use with delivery device 1800. As shown, at left, the cross-sectional profile of cannula 1910c has an elliptical shape; at center, the cross-sectional profile of injection cannula 1910d has a pill or rounded-rectangle shape; at right, the cross-sectional profile of injection cannula 1910e has a crescent or “u” shape. In all examples, the cross-sectional profiles of the injection cannulas 1910c-1910e further depict a channel 1911 disposed through the injection cannula, through which an injection needle, e.g., injection needle 1912, extends through. Please note that these cross-sectional profiles in FIG. 19C are only exemplary and that other cross-sectional profiles for injection cannulas, including rectangular profiles, are also contemplated.

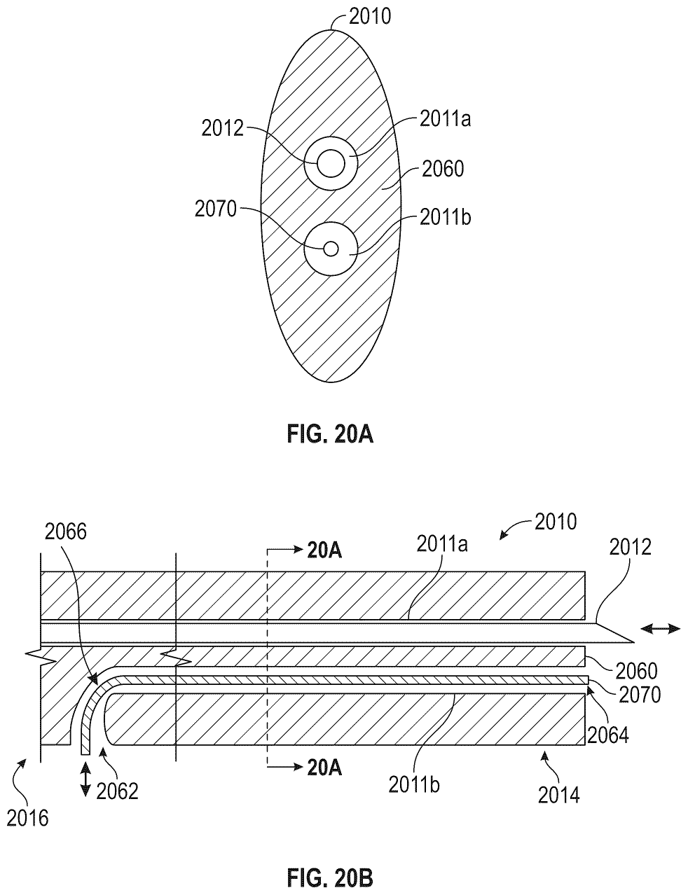

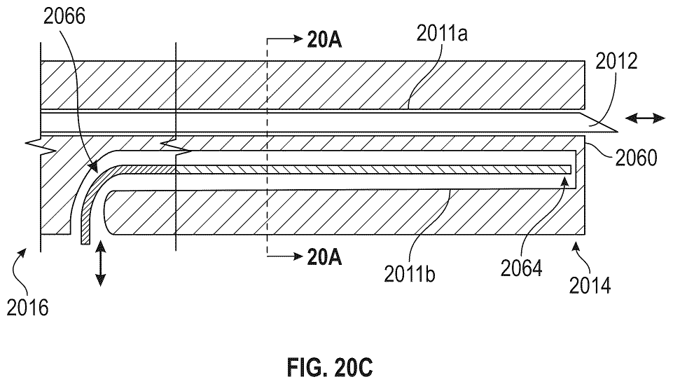

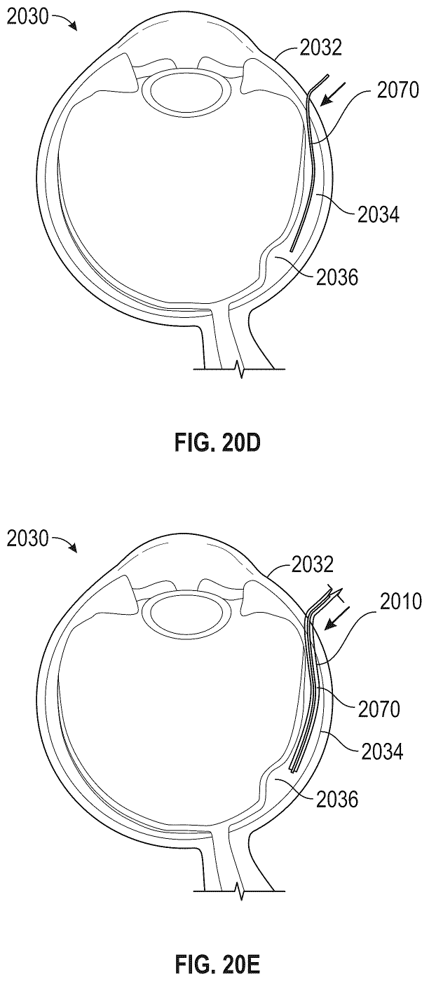

FIGS. 20A-20E illustrate various views of another exemplary injection cannula 2010 which may be used with the delivery devices 1800 and 1801 of FIGS. 18A-18B, or other delivery devices for subretinal injection as described herein, according to certain embodiments of the present disclosure. FIGS. 20A, 20B, and 20C illustrate a cross-sectional top view, a cross-sectional side view, and another cross-sectional side view, respectively, of injection cannula 2010, while FIGS. 20D and 20E illustrate transverse sectional views of an eye at different steps of performing a subretinal injection with the injection cannula 2010. Aspects of the injection cannula 2010 may be combined with other delivery devices and/or components described herein without limitation.

Turning now to FIGS. 20A-20C, the tubular injection cannula 2010 comprises a distal end 2014 and a proximal end 2016. The proximal end 2016 may be coupled to a handle of any suitable delivery device, such as handle 1802 of delivery device 1800 described above. The injection cannula 2010 further comprises a first channel 2011a and a second channel 2011b formed therein, wherein each of the channels 2011a and 2011b extend from the distal end 2014 or substantially near the distal end 2014, to the proximal end 2016, or substantially near the proximal end 2016. As shown, the channels 2011a and 2011b may be separated by one or more walls 2060 of the injection cannula 2010, or by any other suitable means to form two distinct channels in the injection cannula 2010.

An injection needle 2012 is slidably coupled within the first channel 2011a and may terminate proximally within the injection cannula 2010, or within the handle at the proximal end 2016 of the injection cannula 2010. Generally, the injection needle 2012 is fluidically coupled, directly or indirectly via fluidic tubing and/or other connectors, to a fluid source for providing injection fluids (e.g., non-treatment and/or treatment solutions) to the injection needle 2012 for subretinal injection.

Meanwhile, the second channel 2011b may be configured to receive or sheath a wire 2070. The wire 2070 may be utilized as a guide wire and/or a stiffening wire during performance of subretinal injections utilizing a suprachoroidal approach. For example, in certain examples, the wire 2070 is configured as a guide wire to guide the injection cannula 2010 through the suprachoroidal space to a desired position for injection. In such embodiments, the channel 2011b may have an open distal end 2064 to receive the wire 2070 as the injection cannula 2010 is pushed through the suprachoroidal space (shown in FIG. 20C). Further, the channel 2011b may be connected to a port 2062 disposed through an outer wall of the injection cannula 2010 near the proximal end 2016 of the injection cannula 2010, and through which the wire 2070 may be removed from the channel 2011b after the injection cannula 2010 has been placed in a final position for subretinal injection. In certain embodiments, a proximal end 2066 of the channel 2011b may be open to an inner cavity of the handle coupled to the injection cannula 2010, and the wire 2070 may be removed through the handle after the injection cannula 2010 is placed in the final position for subretinal injection. Utilization of the wire 2070 as a guide wire is described in further detail below with reference to FIGS. 20D and 20E.

In certain embodiments, the wire 2070 is configured as a stiffening wire to increase a stiffness of the injection cannula 2010. For example, the wire 2070 may be made of a material having a greater stiffness than that of the injection cannula 2010, and may be inserted into the injection cannula 2010 to reduce the flexibility thereof during insertion and movement of the injection cannula 2010 through the suprachoroidal space. In such embodiments, the channel 2011b may have a closed distal end 2064 to maintain the wire 2070 in the channel 2011b as the injection cannula 2010 is moved through the suprachoroidal space (shown in FIG. 20D). Further, in such embodiments, the channel 2011b may also be connected to the port 2062 disposed near the proximal end 2016 of the injection cannula 2010. Thus, prior to inserting the injection cannula 2010 into a patient's eye and translating the injection cannula 2010 through the suprachoroidal space, the wire 2070 may be inserted into the channel 2011b via the port 2062 to provide increased stiffness. And, as described above, after the injection cannula 2010 has been placed in a final position for subretinal injection, the wire 2070 may be removed from the channel 2011b through the port 2062, or removed along with the injection cannula 2010 after injection. In other embodiments, the proximal end 2066 of the channel 2011b may be open to an inner cavity of the handle coupled to the injection cannula 2010, and the wire 2070 may be inserted into and/or removed from the channel 2011 through the handle.

Generally, the wire 2070 may comprise any suitable material for performing guidance and/or stiffening functions as described herein. For example, in certain embodiments, the wire 2070 comprises a metallic material, such as stainless steel, aluminum, nitinol, or other metal alloys. In certain other embodiments, the wire 2070 comprises a thermoplastic polymer, such polyether ether ketone (PEEK), polyetherketone (PEK), and polytetrafluoroethylene (PTFE).

Referring now to FIGS. 20D and 20E, an exemplary method of utilizing the wire 2070 as a guide wire for subretinal injection is depicted. In FIG. 2D, the wire 2070 is inserted into the patient's eye 2030 through the sclera 2032, and guided through the suprachoroidal space (SCS) 2034 to a target injection site 2036. Once a distal end of the wire 2070 in the suprachoroidal space 2034 is positioned adjacent to the target injection site 2036 in the subretinal space 2034, the injection cannula 2010 is inserted over the wire 2070 such that the wire 2070 is received in the channel 2011b, and the injection cannula 2010 is slid over the wire 2070 until the distal end 2014 is disposed adjacent the target injection site. At this point, the wire 2070 may be removed from the channel 2011b, such as through the port 2062 or through the handle of the delivery device coupled to the injection cannula 2010, or the wire 2070 may remain in the channel 2011b as the injection is carried out via the injection needle 2012.

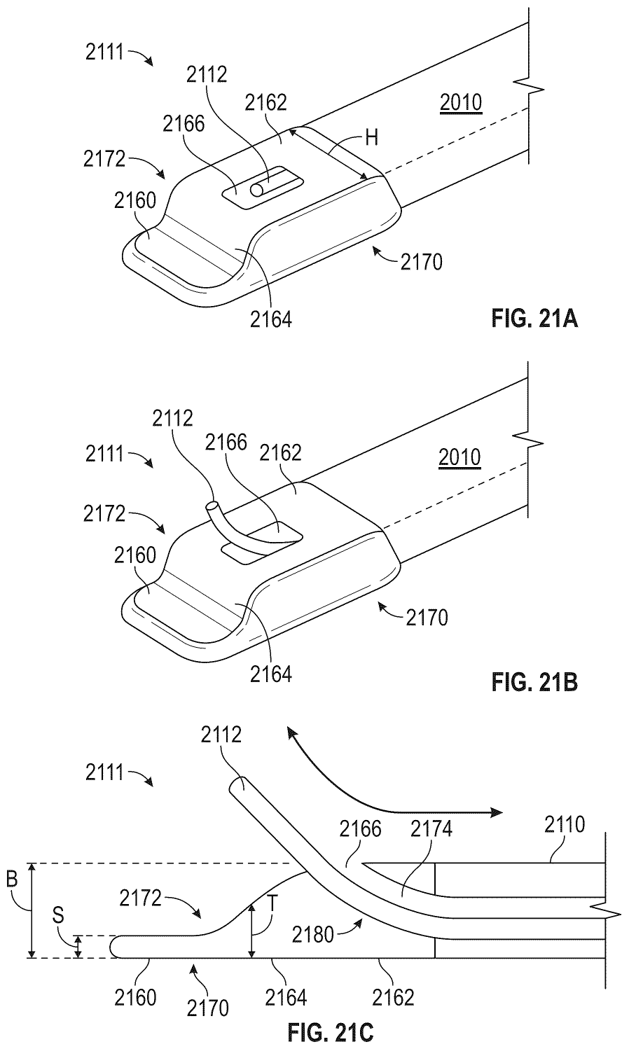

FIGS. 21A-21C illustrate various views of an exemplary distal tip 2111 of an injection cannula, according to certain embodiments of the present disclosure. More particularly, FIGS. 21A and 21B illustrate perspective views of the distal tip 2111, while FIG. 21C illustrates a schematic cross-sectional side view of the distal tip 2111. The distal tip 2111 is an exemplary distal tip of delivery devices 1800 and 1801 of FIGS. 18A-18B, or other delivery device and/or injection cannula for subretinal injection as described herein. Aspects of the distal tip 2111 may be combined with other delivery devices and/or components described herein without limitation.

As shown in FIGS. 21A-21C, distal tip 2111 comprises a distal spatula portion 2160 and a proximal body portion 2162. In certain embodiments, the distal tip 2111 further comprises a transition portion 2164 disposed between and coupling the spatula portion 2160 and the body portion 2162. The body portion 2162 proximally couples to an injection cannula 2110 of a delivery device.

In certain embodiments, the spatula portion 2160 has a vertical dimension S (shown in FIG. 21C), which is smaller than a vertical dimension B of the body portion 2162. In certain embodiments, the vertical dimension S and/or the vertical dimension B are uniform or substantially uniform along a longitudinal length (e.g., a length parallel to the major longitudinal axis A of the injection cannula) of the spatula portion 2160 and/or body portion 2162, respectively. The reduced vertical dimension S of the spatula portion 2160 facilitates delamination (i.e., separation) of the choroid and the sclera as the distal tip 2111 is moved through the suprachoroidal space to a target subretinal injection site. Meanwhile, the increased vertical dimension B of the body portion 2162 facilitates sheathing of an extendable injection needle 2112 within the body portion 2162, which may be configured to extend from and retract into a port 2166 formed in the body portion 2162.

In certain embodiments, the transition portion 2164 has a varying vertical dimension T that increases proximally from the spatula portion 2160 to the body portion 2162. In other words, the vertical dimension T of the transition portion 2164 tapers from the body portion 2162 to the spatula portion 2162. In certain embodiments, the vertical dimension T of the transition portion 2163 linearly varies across a longitudinal length of the transitional portion 2164. In certain embodiments, the vertical dimension T of the transition portion 2164 non-linearly varies along a longitudinal length of the transitional portion 2164, and thus, the transition portion 2164 may have a curvature along the longitudinal length thereof. The varying vertical dimension T of the transition portion 2164 creates a more gradual increase in vertical thickness between the spatula portion 2160 and the body portion 2162, thereby providing a more gradual increase in stress against the choroid and sclera as the as the distal tip 2111 is moved through the suprachoroidal space and reducing damage to such tissues as a result of such movement.

Generally, while the distal tip 2111 may comprise multiple varying vertical dimensions (e.g., S, T, and B), the distal tip 2111 may, in certain embodiments as shown in FIGS. 21A and 21B, comprise a uniform horizontal lateral (e.g., perpendicular to a major longitudinal axis A of the distal tip 2111 and/or cannula 2010) dimension H along a longitudinal length of the distal tip 2111. In certain other embodiments, however, the distal tip 2111 may comprise two or more different horizontal lateral dimensions H along the longitudinal length of the distal tip 2111.

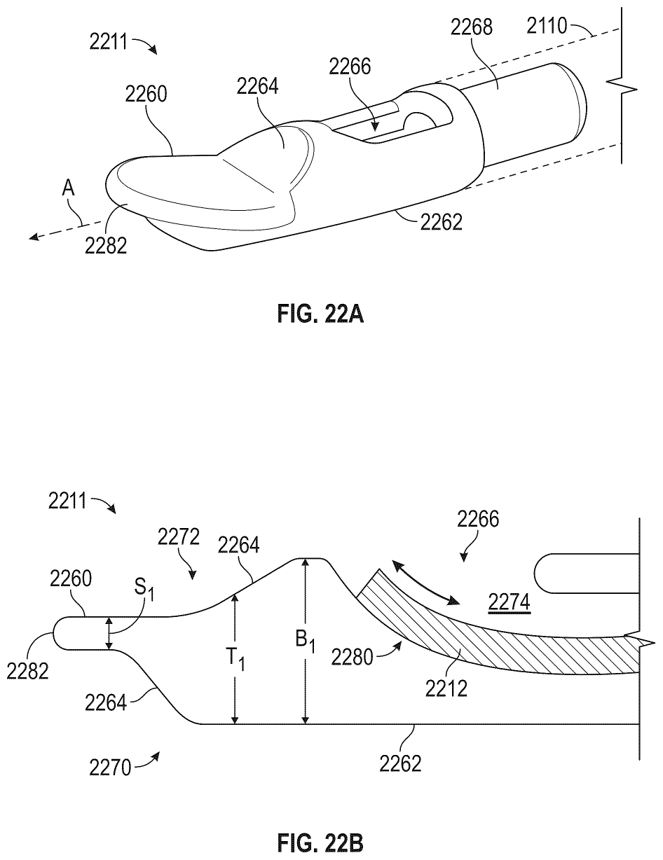

Turning back now to FIG. 21C, a first side 2170 of the distal tip 2111 may be substantially planar and coplanar with the injection cannula 1810, while a second side 2172 of the distal tip 2111 opposite the first side 2170 may have a stepped, sloped, tapered, or other varying cross-sectional side profile (e.g., along the longitudinal length of the distal tip 2111) as a result of the different vertical dimensions of the spatula portion 2160, transition portion 2164, and body portion 2162. In still other embodiments, as described below in FIGS. 22A and 22B, the first side 2170 may have also have a stepped, sloped, tapered, or other varying profile, which may be identical or near identical to the second side 2172. When moving the distal tip 2111 through the suprachoroidal space, the distal tip 2111 is oriented such that the first side 2170 faces the sclera (away from the choroid), while the second side 2172 faces the choroid.

As further shown in FIG. 21C, an interior lumen 2174 of the distal tip 2111 may have a sloped surface 2180 that distally increases in distance from the first side 2170. This sloped surface 2180 may act as a “ramp” to facilitate extension of the injection needle 2112 from the distal tip 2111 in a direction that is non-parallel to the major longitudinal axis A of the injection cannula 2110, which is necessary for suprachoroidal subretinal injections as the injection needle must pierce through the choroid disposed along the suprachoroidal space (thus, the injection needle 2112 must extend in a direction that is tangential to the major longitudinal axis A of the injection cannula 2110). Accordingly, when the injection needle 2112 disposed within the injection cannula 2110 is extended through the distal tip 2111, the sloped surface 2180 causes the injection needle 2112 to bend upwards, away from the first side 2170, and slide along the sloped surface 2180 until the injection needle 2112 passes through the port 2166. To facilitate the upward bending of the needle 2112, the needle may comprise a flexible material, such as nitinol, polyimide, or other suitable flexible surgical-grade materials.

FIGS. 22A and 22B illustrate various views of an exemplary distal tip 2211 of an injection cannula, according to certain embodiments of the present disclosure. More particularly, FIG. 22A illustrates a perspective view of the distal tip 2211, while FIG. 22B illustrates a schematic cross-sectional side view of the distal tip 2211. The distal tip 2211 is another exemplary distal tip for delivery devices 1800 and 1801 of FIGS. 18A-18B, or other delivery devices and/or injection cannulas for subretinal injection as described herein. Aspects of the distal tip 2211 may be combined with other delivery devices and/or components described herein without limitation.

Similar to distal tip 2111, distal tip 2211 comprises a distal spatula portion 2260, a proximal body portion 2262, and a transition portion 2264 disposed between and coupling the spatula portion 2260 and the body portion 2262. In certain embodiments, the body portion 2262 proximally couples to an injection cannula 2110 (shown in phantom in FIG. 22A) of a delivery device. In certain embodiments, the body portion 2262 is proximally coupled to a connector 2268, which may be configured to be inserted into and friction fit with a distal end of the injection cannula 2110 for coupling the distal tip 2211 thereto.

As shown in FIG. 22A, the spatula portion 2260 may have a substantially semi-circular and disc-like shape with a rounded edge 2282. The rounded edge 2282 and semi-circular disc-like shape of the spatula portion 2260 facilitate easier delamination of the choroid from the sclera, with reduced damage to such tissues, as the distal tip 2211 is moved through the suprachoroidal space to a target injection site. In certain embodiments, the spatula portion 2260 comprises a substantially flat (or planar) disc-like shape; in certain other embodiments, the spatula portion 2260 comprises a curved disc-like shape that substantially matches a curvature of the suprachoroidal space. Meanwhile, the body portion 2262 may have a cylindrical, or substantially cylindrical in shape, and the transition portion 2264 may have a triangular or ramp-like shape between the spatula portion 2260 and the body portion 2262.

The semi-circular disc-like spatula portion 2260 has a vertical dimension S₁, which is smaller than a vertical dimension B₁ of the cylindrical body portion 2262. In certain embodiments, the vertical dimension S₁ and/or the vertical dimension B₁ are uniform or substantially uniform along a longitudinal length (e.g., a length parallel to the major longitudinal axis A of the injection cannula) of the spatula portion 2260 and/or body portion 2262, respectively. The reduced vertical dimension S₁ of the spatula portion 2260, in tandem with the rounded edge 2282 and disc-like shape thereof, facilitates easier delamination of the choroid and the sclera. Meanwhile, the increased vertical dimension B₁ of the body portion 2262 facilitates the housing of an extendable injection needle 2212 within the body portion 2262, which may be configured to extend from and retract into a port 2266 formed in the body portion 2262.

In certain embodiments, the transition portion 2264 has a varying vertical dimension Ti that increases proximally from the disc-like spatula portion 2260 to the cylindrical body portion 2262. In certain embodiments, the vertical dimension Ti of the transition portion 2264 linearly varies across a longitudinal length of the transitional portion 2264. In certain embodiments, the vertical dimension Ti of the transition portion 2264 non-linearly varies along a longitudinal length of the transitional portion 2264, and thus, the transition portion 2264 may have a curvature along the longitudinal length thereof. The varying vertical dimension Ti of the transition portion 2264 creates a more gradual increase in vertical thickness between the spatula portion 2260 and the body portion 2262, thereby providing a more gradual increase in stress against the choroid and sclera as the as the distal tip 2211 is moved through the suprachoroidal space to and thereby reducing damage to such tissues.

Turning now to FIG. 22B, both of a first side 2270 and a second side 2272 of the distal tip 2211 have a stepped, sloped, tapered, or other varying cross-sectional side profile (e.g., along the longitudinal length of the distal tip 2211) as a result of the different shapes and thicknesses of the spatula portion 2260, transition portion 2264, and body portion 2262. In certain embodiments, the cross-sectional side profiles of the first side 2270 and the second side 2272 are identical. In certain other embodiments, the cross-sectional side profiles of the first side 2270 and the second side 2272 are different. For example, as shown in the embodiments of FIG. 22B, the transition portion 2264 may have a steeper slope between the spatula portion 2160 and the body portion 2262 on the first side 2270, and a more gradual slope between the spatula portion 2160 and the body portion 2262 on the second side 2272. Such differences in the profiles of the first side 2270 and the second side 2272 may be utilized to account for the different fragilities of the choroid and sclera. For example, when the distal tip 2211 is inserted and moved through the suprachoroidal space, the distal tip 2211 is oriented such that the first side 2270 faces the sclera (away from the choroid), while the second side 2272 faces the choroid. Because the choroid is more delicate than the sclera, and the sclera is more robust than the choroid, the first side 2270 may have a steeper transition between the spatula portion 2260 and the body portion 2262, whereas the second side 2272 may have a more gradual transition between the spatula portion 2260 and the body portion 2262.

As further shown in FIG. 22B, similar to the distal tip 2111 described above, an interior lumen 2274 of the distal tip 2211 may also have a sloped surface 2280 that distally increases in distance from the first side 2270. This sloped surface 2280 may act as a ramp to facilitate extension of the injection needle 2212 from the distal tip 2211 in a tangential direction relative to the major longitudinal axis A of the injection cannula 2210. Accordingly, when the extendable injection needle 2212 disposed within the injection cannula 2210 is extended through the distal tip 2211, the sloped surface 2280 causes the injection needle 2212 to bend upwards, away from the first side 2270, and slide along the sloped surface 2280 until the injection needle 2212 passes through the port 2266. In certain embodiments, to facilitate the upward bending of the needle 2212, the needle may comprise a flexible material, such as nitinol, polyimide, or other suitable flexible surgical-grade materials.

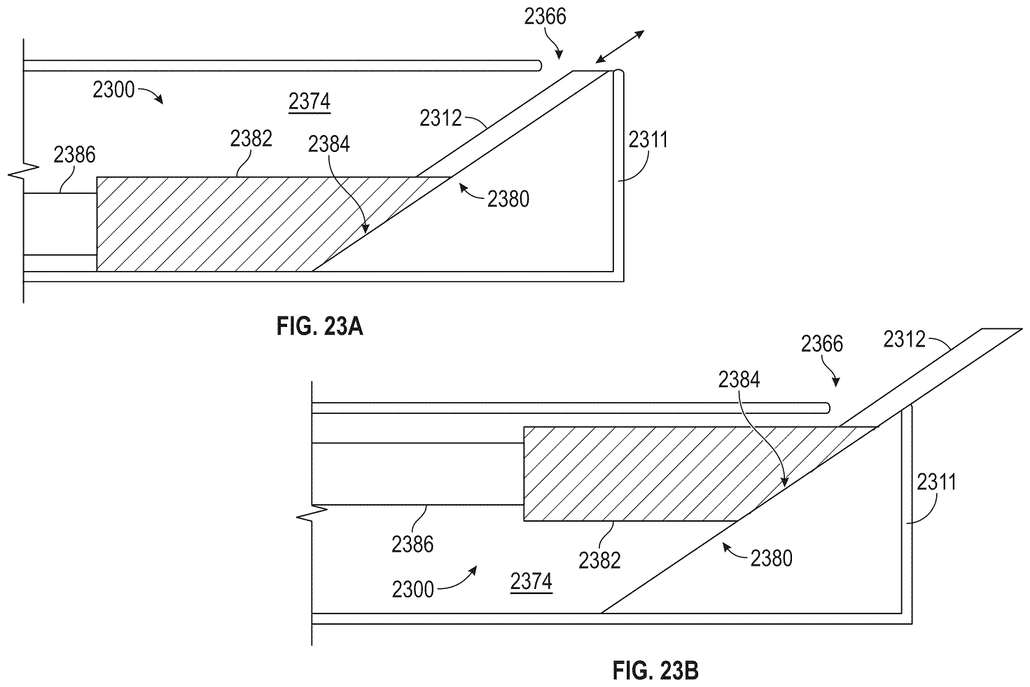

FIGS. 23A and 23B illustrate cross-sectional side views of an exemplary internal ramp assembly 2300 for a distal tip of an injection cannula for a subretinal delivery device, according to certain embodiments of the present disclosure. The ramp assembly 2300 may be utilized in combination with any of the distal tips described herein, including distal tips 2111 and 2211 described above, to facilitate the extension of an injection needle from the distal tip in a direction tangential, or non-parallel, to the major longitudinal axis of the corresponding injection cannula.

As shown in FIGS. 23A and 23B, an interior lumen 2374 of a distal tip 2311 may comprise a sloped surface 2380 distally terminating at a port 2366 of the distal tip 2311. In other embodiments described herein, such sloped surface 2380 may be utilized to directly guide and “bend” a more conventional injection needle upwards and out of the port 2366. However, in the current embodiments, an injection needle 2312 is proximally coupled to a sliding block 2382 that instead interfaces with the sloped surface 2380 of the distal tip 2311 to facilitate extension and/or retraction of the injection needle 2312 through the port 2366. In certain embodiments, the sliding block 2382 is fabricated from materials that facilitate easy sliding of the injection needle 2312 with reduced frictional resistance, such as steel, titanium, PEEK (polyetheretherketone), polyoxymethylene (POM), polytetrafluoroethylene (PTFE), combinations thereof, or the like.

The sliding block 2382 is proximally coupled to an inner fluidic shaft 2386, which may extend through an injection cannula of a delivery device to a handle thereof. The inner fluidic shaft 2386 fluidically couples, directly or indirectly, the sliding block 2382 and injection needle 2312 to a fluid source or fluidic tubing connected to a fluid source. Accordingly, in certain embodiments, the sliding block 2382 comprises a fluid channel, which facilitates the flow of injection fluids from the inner fluidic shaft 2386 to the injection needle 2312 fluidically coupled to the sliding block 2382 for delivery to a target subretinal injection site. The inner fluidic shaft 2386 may further be coupled to an actuator or other control mechanism disposed in the handle of a delivery device, which may enable manual actuation of the inner fluidic shaft 2386, by a user, in a proximal direction or distal direction through the injection cannula of the delivery device.

In certain embodiments, the sliding block 2382 itself comprises a distal sloped surface 2384 that corresponds with (e.g., matches or mates with) the sloped surface 2380. In certain embodiments, the sloped surface 2384 may be disposed at the same or a substantially similar angle as the sloped surface 2380 relative to a major longitudinal axis of the distal tip 2311 or an injection cannula coupled to the distal tip 2311. Upon application of a distally-directed force (pushing force) on the sliding block 2382 from the proximal inner fluidic shaft 2386, the sloped surface 2384 may interact with the sloped surface 2380 such that the sliding block 2382 translates (e.g., slides) upward along the sloped surface 2380, thereby extending the injection needle 2312 through the port 2366. FIG. 23B illustrates the ramp assembly 2300 in an “extended” position. Similarly, upon application of a proximally-directed (pulling) on the sliding block 2382 by the inner fluidic shaft 2386, the sloped surface 2384 may interact with the sloped surface 2380 such that the sliding block 2382 translates (e.g., slides) downward along the sloped surface 2380, thereby retracting the injection needle 2312 through the port 2366. FIG. 23A illustrates the ramp assembly 2300 in a “retracted” position. Note that although the distal sloped surface 2384 and the sloped surface 2380 are depicted as planar “ramp-like” surfaces, other morphologies for such surfaces are also contemplated, including non-planar and/or curved surfaces. It is further contemplated that sloped surface 2384 may have a different morphology as compared to sloped surface 2380—for example, sloped surface 2384 may be rounded or curved, while sloped surface 2380 may be planar.

The described mechanism facilitates the extension and retraction of the injection needle 2312 through the port 2366 without requiring bending of the injection needle 2312. Accordingly, stiffer materials may be utilized for the injection needle 2312, in addition to flexible materials such as polyimide, nitinol, etc. For example, in certain embodiments, the injection needle 2312 may comprise a metallic material such as aluminum, stainless steel, nitinol, and other metallic alloys. In further embodiments, the injection needle 2312 may comprise a thermoplastic polymeric material, such as polyimide, polyether ether ketone (PEEK), polyetherketone (PEK), and polytetrafluoroethylene (PTFE).

Generally, the injection needle 2312 may be coupled to the sliding block 2382 at any desired angle or orientation. In the examples of FIGS. 23A and 23B, the injection needle 2312 is coupled to the sliding block 2382 at an angle substantially matching that of the sloped surfaces 2380 and 2384.

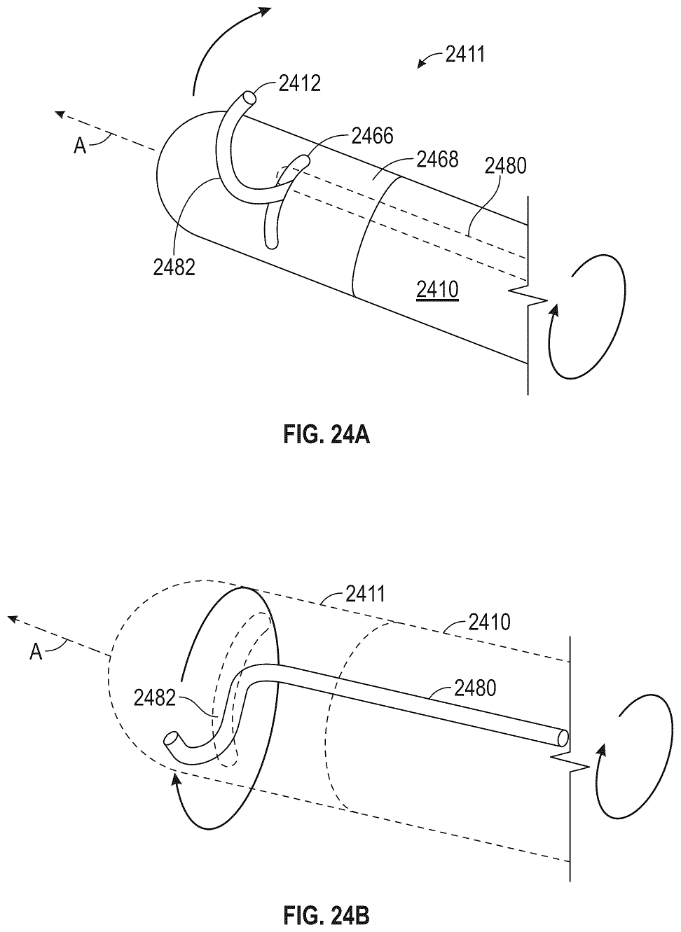

FIGS. 24A and 24B illustrate schematic perspective views of another exemplary distal tip 2411 of an injection cannula 2410 for a delivery device, according to certain embodiments of the present disclosure. The distal tip 2411 may be another exemplary distal tip for delivery devices 1800 and 1801 of FIGS. 18A-18B, or other delivery devices and/or injection cannulas for subretinal injection as described herein. Aspects of the distal tip 2411 may therefore be combined with other delivery devices and/or components described herein without limitation.

Turning to FIG. 24A, the distal tip 2411 comprises a slotted port 2466 disposed through a sidewall 2468 of the distal tip 2411. The slotted port 2466 may be oriented such that a length of the slotted port 2466 along the circumference of the distal tip 2411 is greater than a width of the slotted port 2466 in a longitudinal direction (e.g., a parallel to the major longitudinal axis A of the injection cannula 2410).

An injection needle 2412 is disposed within, and extends through, the distal tip 2411 and the injection cannula 2410. As shown in FIG. 24B, the injection needle 2412 comprises a first, prolonged portion 2480 which may extend proximally through an entire length of the injection cannula 2410 and couple to an actuator or other suitable control mechanism disposed on a handle of a delivery device coupled to the injection cannula 2410. The injection needle 2412 further comprises a second, corkscrew portion 2482 disposed at a distal end of the injection needle 2412. The corkscrew portion 2482 comprises a portion of the injection needle 2412 that is preformed to bend, or curl, along a plane perpendicular to a major longitudinal axis of the prolonged portion 2480 such that the corkscrew portion 2482 resembles a corkscrew or helical shape. The corkscrew portion 2482 is configured to extend from, and retract into, the slotted port 2466 upon rotation of the prolonged portion 2480 by, e.g., the actuator or other control mechanism on the delivery device handle. For example, in certain embodiments, the control mechanism may comprise a rotating knob or dial on a delivery device handle, and rotation of the knob or dial by a user may cause rotation of the prolonged portion 2482, thereby causing the corkscrew portion 2482 to rotate about an axis of the prolonged portion 2482 and extend from the slotted port 2466.

Similar to the examples of FIGS. 24A and 24B, the described mechanism facilitates efficient extension and retraction of the injection needle 2412 through the slotted port 2466 at a tangential angle relative to the injection cannula 2410, without requiring active bending of the injection needle 2412. Accordingly, stiffer materials may be utilized for the injection needle 2412, in addition to flexible materials such as polyimide, nitinol, etc. For example, in certain embodiments, the injection needle 2412 may comprise a metallic material such as aluminum, stainless steel, and other metallic alloys. In further embodiments, the injection needle 2312 may comprise a thermoplastic polymeric material, such as polyether ether ketone (PEEK), polyetherketone (PEK), and polytetrafluoroethylene (PTFE).

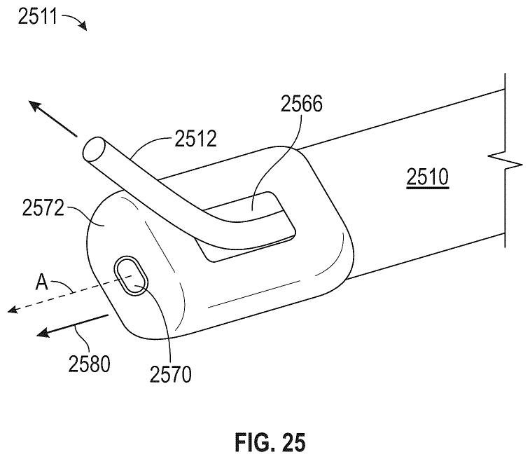

FIG. 25 illustrates a schematic perspective view of an exemplary distal tip 2511 of an injection cannula 2510 for a delivery device, according to certain embodiments of the present disclosure. The distal tip 2511 is another exemplary distal tip for delivery devices 1800 and 1801 of FIGS. 18A-18B, or other delivery devices and/or injection cannulas for subretinal injection as described herein. Aspects of the distal tip 2511 may therefore be combined with other delivery devices and/or components described herein without limitation.

As shown, the distal tip 2511 comprises a port 2566 in a sidewall 2568 thereof for enabling ingress and egress of an extendable injection needle 2512. In certain embodiments, the distal tip 2511 comprises a sloped surface, or ramp, within an interior lumen thereof that distally terminates at the port 2566 and facilitates the extension of the injection needle 2512 from the port 2566 at a tangential angle relative to a major longitudinal axis A of distal tip 2511 and/or injection cannula 2510.

In addition to the port 2566 through the sidewall 2568, the distal tip 2511 further comprises a fluid port 2570 disposed through a distal surface 2572 of the distal tip 2511. The fluid port 2570 may be fluidly coupled to a fluid line or fluid flow path extending through the distal tip 2511, the injection cannula 2510, and/or a handle coupled to the injection cannula 2510. The fluid line or flow path may comprise, for example, flexible fluidic tubing, such as the fluidic tubing described elsewhere herein for supplying injection fluids to an injection needle, e.g., injection needle 2512. Generally, the fluid line or flow path coupled to the fluid port 2570 may further be fluidly connected to a fluid source for providing a hydro-dissection fluid, such as balanced salt solution (BSS) or other suitable fluid. During use, the hydro-dissection fluid may be flowed through the fluid line or flow path and out of the fluid port 2570 while the distal tip 2511 is moved through the suprachoroidal space to delaminate, or separate, the choroid from the sclera and make positioning of the distal tip 2511 at the target injection site easier for the user. Accordingly, in certain embodiments, the fluid port 2570 is positioned through the distal surface 2572 of the distal tip 2511 such that hydro-dissection fluid is flowed out of the distal surface 2572 in a direction 2580 parallel or substantially parallel to the major longitudinal axis A of distal tip 2511 and/or injection cannula 2510.

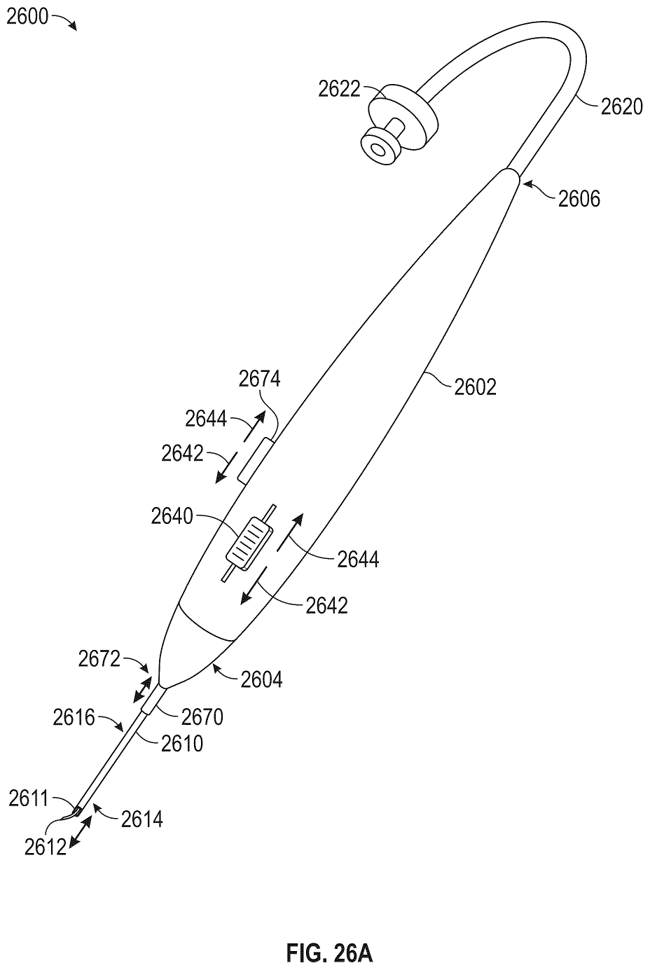

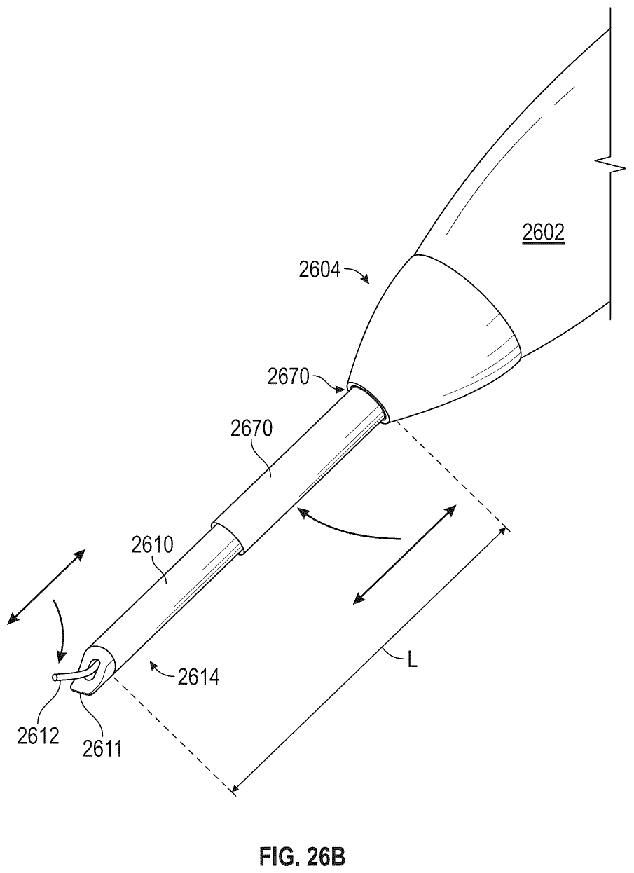

Referring now to FIGS. 26A and 26B, another exemplary subretinal delivery device 2600 is illustrated in various perspective views, according to certain embodiments of the present disclosure. The delivery device 2600 is substantially similar to delivery device 1800, and may be used as, e.g., the delivery device 414 of the surgical system 400 of FIG. 4. Aspects of the delivery device 2600 may be combined with other delivery devices and/or components described herein without limitation. Certain aspects of the delivery device 2600 are particularly beneficial for performing subretinal injections (and other related procedures) via a suprachoroidal approach, as described above with reference to FIG. 3.

As shown, the delivery device 2600 includes a handle 2602 and a tubular injection cannula 2610 having a proximal end 2616 coupled to and extending distally from a distal end 2604 of the handle 2602. A distal end 2614 of the injection cannula 2610 comprises a distal tip 2611, which may be tapered or sloped relative to a major longitudinal axis of the injection cannula 2610 in order to facilitate separation of the choroid from the sclera as the injection cannula 2610 is moved through the suprachoroidal space. In certain embodiments, the distal tip 2611 may have oval-like, widened, or flattened cross-section, to facilitate easier translation through the suprachoroidal space. Exemplary distal tips are discussed in more detail elsewhere herein. The injection cannula 2610 and/or distal tip 2611 are generally formed of any suitable flexible surgical-grade materials, such a flexible metallic or thermoplastic polymeric materials. Examples of flexible metallic materials include nitinol and other metallic alloys. Examples of suitable thermoplastic polymeric materials include polyimide. In certain embodiments, the distal tip 2611 is formed of a rigid material, and the remainder of the injection cannula 2610 is formed of a flexible material.

As further shown in FIGS. 26A and 26B, a curved or straight injection needle 2612 is disposed within the injection cannula 2610 for piercing desired ocular tissues (here, the choroid and RPE) at an angle relative to a major longitudinal axis of the injection cannula 2610 to deliver a fluid to the subretinal space. In exemplary embodiments, the injection cannula 2610 is a 23-, 25-, or 27-gauge needle, while the injection needle 2612 is a finer gauge needle, such as a 38-gauge needle. However, other sizes/gauges of injection cannulas and injection needles may be used in other embodiments. In certain embodiments, as described elsewhere herein, the injection needle 2612 is formed of a flexible material, such as nitinol or polyimide. In certain embodiments, the injection needle 2612 is formed of a stiff material, including metallic materials such as stainless steel or thermoplastic polymers such as polyether ether ketone (PEEK), polyetherketone (PEK), and polytetrafluoroethylene (PTFE).

In certain embodiments, the injection needle 2612 is configured to slidably extend from and retract into a distal end 2614 of the injection cannula 2610, which facilitates the prevention of damage to the injection needle 2612 during insertion and/or movement of the injection cannula 2610 in the patient's eye. Such actuation of the injection needle 2612 may be controlled by any suitable control mechanism. In FIG. 26A, actuation of the injection needle 2612 is controlled by a first toggle 2640 on the handle 2602. In certain embodiments, the toggle 2640 comprises a sliding button or switch, wherein sliding of the toggle 2640 by a user (e.g., a surgeon) in a distal direction 2642 causes the injection needle 2612 to extend from the injection cannula 2610, and sliding of the toggle 2640 in a proximal direction 2644 causes the injection needle 2612 to retract into the injection cannula 2610. In certain embodiments, the injection needle 2612 is coupled to an inner fluidic shaft at least partially disposed within the cannula 2610 for fluidically coupling the injection needle 2612 to fluidic tubing or for coupling the injection needle 2612 to the toggle 2640. In such embodiments, the inner fluidic shaft may be slidably disposed within the cannula 2610 to facilitate extension and retraction of the injection needle 2612 upon actuation of the toggle 2640.

In certain embodiments, a sliding toggle 2640 may also be lockable, such that the injection needle 2612 may be fixed in either an extended or a retracted position. Locking of the injection needle 2612 prevents unintended movement of the injection needle 2612 during a retinal procedure, e.g., a subretinal injection, thereby reducing the risk of unwanted tissue damage and improving the overall safety of such procedures. In one example, to unlock/release the sliding toggle 2640 for adjustment, the toggle 2640 may be continuously depressed by a user, allowing the user to freely slide the toggle 2640 and thus, freely extend or retract the injection needle 2612. In this example, the toggle 2640 may only be movable while depressed (e.g., activated) by the user. Correspondingly, releasing the toggle 2640 may cause the toggle 2640 to raise and lock in place, thereby locking the injection needle 2612 in place. Such a push button locking mechanism may be facilitated, in part, by a spring lever disposed with the handle 2602, as well as one or more tracks comprising grooves or notches along which the toggle 2640 may slide.

As further shown in FIG. 26A, in certain embodiments, a flexible fluidic tubing 2620 for supplying injection fluids (e.g., non-treatment and/or a treatment solutions) to the delivery device 2600 may be disposed through a proximal end 2606 of the handle 2602 and fluidically coupled to the injection needle 2612 within the handle 2602. In certain embodiments, the fluidic tubing 2620 may couple to the proximal end 2606 of the handle 2602, or another fluidic tubing within the handle 2602 (described elsewhere herein). Generally, the fluidic tubing 2620 comprises a supply line through which injection fluids non-treatment and/or a treatment solutions from a fluid source (not shown in FIG. 26A) may be provided to the delivery device 2600 for delivery to an eye. In certain embodiments, the fluid source comprises a fluidic system, which may be coupled to the fluidic tubing 2620 via connection 2622, such as a Luer lock or other male-female coupling. In certain other embodiments, the handle 2602 may comprise an actuatable internal chamber (not shown in FIG. 26A) fluidically coupled to the injection cannula 2610 and containing the injection fluids. In such embodiments, subretinal delivery device 2600 may not be coupled to any external fluidic tubing.

In further embodiments, to simplify fluidic preparation for subretinal injection and/or the injection itself, a therapeutic agent may be provided to the delivery device 2600 from a prefilled cartridge that can be coupled directly to the delivery device 2600, or to a fluidic system connected to the delivery device 2600 via the fluidic tubing 2620. Cartridges for therapeutic agents are described in more detail elsewhere herein.

The delivery device 2600 further includes a stiffener sleeve 2670. The stiffener sleeve 2670 is slidably coupled to the injection cannula 2610 and, in the embodiments of FIGS. 26A and 26B, substantially surrounds at least a portion of the injection cannula 2610. However, in certain other embodiments, the stiffener sleeve 2700 may be disposed within the injection cannula 2610 and function substantially similarly.

The stiffener sleeve 2670 is adjustable relative to the injection cannula 2610, enabling a user to manually position the stiffener sleeve 2670 (e.g., a distal end of the stiffener sleeve 2670) at different points along a length L (shown in FIG. 26B) of the injection cannula 2610 exterior to the handle 2602. Accordingly, a user may selectively adjust (e.g., increase or decrease) a level of stiffness of a portion of the injection cannula 2610 by adjusting a position of the stiffener sleeve 2670 relative to the distal end 2614 of the injection cannula 2610, thus manipulating the amount of support provided to the injection cannula 2610 and stabilizing the injection cannula 2610 during use thereof. The stiffener sleeve 2670 thus enables the option of increasing the stiffness of injection cannula 2610 for easier entry and access to the suprachoroidal space, while also enabling the option to decrease the stiffness of the injection cannula 2610 once in the suprachoroidal space to facilitate conformation of the injection cannula 2610 with a curvature of the patient's eye, thereby reducing stress on the choroid and sclera. For example, the stiffener sleeve 2670 may be fully extended to increase the stiffness of the injection cannula 2610 before/during entry of the injection cannula 2610 into the suprachoroidal space, thereby facilitating better control and/or maneuverability thereof and improving overall safety and ease-of-use. Once inserted into the suprachoroidal space, the stiffener sleeve 2670 may be retracted to decrease the stiffness of the injection cannula 2610. For example, the stiffener sleeve 2670 may be retracted as the injection cannula 2610 is pushed further into the suprachoroidal space to facilitate a flexible injection cannula 2610 within the suprachoroidal space, thereby reducing stress and damage to the choroid, including the risk of choroidal hemorrhage.

The stiffener sleeve 2670 is generally a cylindrical and hollow tube substantially surrounding a portion of the injection cannula 2610 at or near the proximal end 2616 thereof. In certain embodiments, the stiffener sleeve 2670 has uniform lateral dimensions along a longitudinal or axial length thereof, thereby resembling a simple cylinder. In certain embodiments, the stiffener sleeve 2670 has non-uniform lateral dimensions along a longitudinal or axial length thereof, and may resemble a tapered cylinder. The stiffener sleeve 2670 is generally formed of a surgical-grade material suitable having suitable stiffness for providing increased stiffness or support to the injection cannula 2610. In certain embodiments, the stiffener sleeve 2670 is formed of a metallic material, such as stainless steel, aluminum, or titanium. In certain embodiments, the stiffener sleeve 2670 is formed of a composite material, such as a thermoplastic polymer composite material or a ceramic composite material. For example, the stiffener sleeve 2670 may comprise polyether ether ketone (PEEK), polyetherketone (PEK), and/or polytetrafluoroethylene (PTFE). In certain embodiments, the stiffener sleeve 2670 comprises polycarbonate (PC).

Along with the injection cannula 2610, the stiffener sleeve 2670 is movably disposed through an opening 2672 in the distal end 2604 of the handle 2602. A proximal end of the stiffener sleeve 2670 is disposed in an interior chamber or lumen of the handle 2602. The stiffener sleeve 2670 is sized to possess a longitudinal (i.e., axial) length sufficient to provide a desired rigidity and stability to the injection cannula 2610 while having a portion thereof still remaining in the interior of handle 2602 when the stiffener sleeve 2670 is in a fully extended or protracted position

As described above, the stiffener sleeve 2670 is configured to slidably extend from and retract into the opening 2672 of the handle 2602. Such actuation of the stiffener sleeve 2670 may be controlled by any suitable control mechanism. In FIG. 26A, actuation of the stiffener sleeve 2670 is shown as being controlled by a second toggle 2674 on the handle 2602. In certain embodiments, the toggle 2674 comprises a sliding button or switch, similar to toggle 2640, wherein sliding of the toggle 2674 by the user in the distal direction 2642 causes the stiffener sleeve 2670 to extend from the opening 2672, and sliding of the toggle 2674 in the proximal direction 2644 causes the stiffener sleeve 2670 to retract into the opening 2672. Alternatively, wherein the toggle 2674 comprises a push button, extension and/or retraction of the stiffener sleeve 2670 may be controlled via depression or release of the toggle 2674.

In certain embodiments, the toggle 2674 may also be lockable, such that the stiffener sleeve 2670 may be fixed in a place along the length L upon adjustment by the user. In certain examples, the toggle 2674 may be lockable in one or more preset positions corresponding to incremental preset positions of the stiffener sleeve 2670 along the length L of the injection cannula 2610, wherein such preset positions of the stiffener sleeve 2670 further correspond to predetermined levels of rigidity for the injection cannula 2610. Locking of the stiffener sleeve 2670 prevents unintended movement of the stiffener sleeve 2670 during a surgical procedure, e.g., a subretinal injection, thereby reducing the risk of accidentally over-stiffening or under-stiffening the injection cannula 2610 while positioning the injection cannula 2610 within the patient's eye. In certain examples wherein the toggle 2674 is a sliding button, to unlock/release the toggle 2674 for adjustment, the toggle 2674 may be continuously depressed by a user, allowing the user to freely slide the toggle 2674 and thus, freely extend or retract the stiffener sleeve 2670. In this example, the toggle 2674 may only be movable while depressed (e.g., activated) by the user. Correspondingly, releasing the toggle 2674 may cause the toggle 2674 to raise and lock in place, thereby locking the stiffener sleeve 2670 in place. Such a locking mechanism may be facilitated, in part, by utilization of a spring lever disposed with the handle 2602, as well as one or more tracks comprising grooves or notches along which the toggle 2674 may slide.

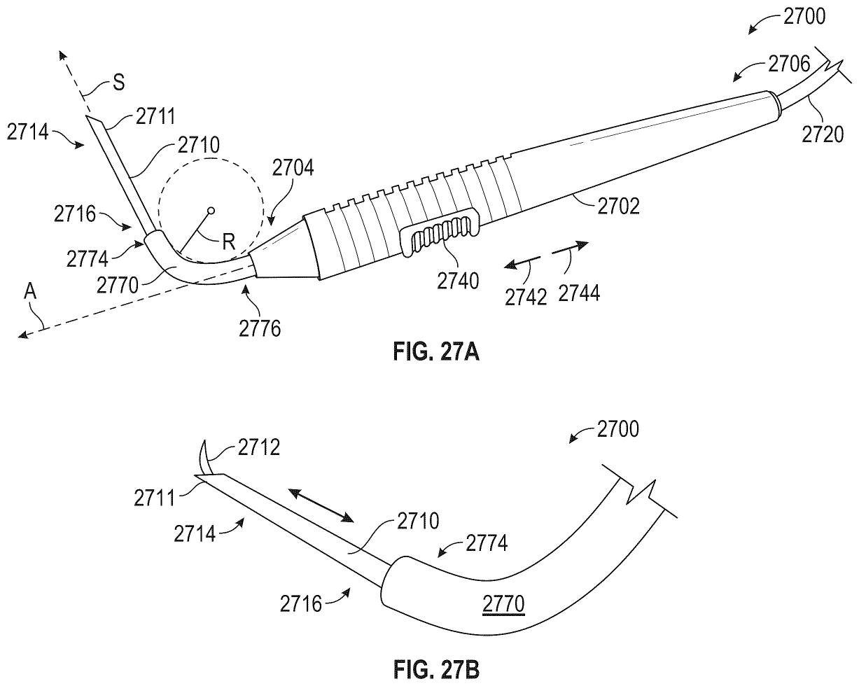

FIGS. 27A and 27B illustrate various perspective views of another exemplary subretinal delivery device 2700, according to certain embodiments of the present disclosure. The delivery device 2600 is substantially similar to delivery devices 1800 and 2600, and may be used as, e.g., the delivery device 414 of the surgical system 400 of FIG. 4. Aspects of the delivery device 2700 may be combined with other delivery devices and/or components described herein without limitation. Similar to the previous delivery device 2600, certain aspects of the delivery device 2700 are particularly beneficial for performing subretinal injections (and other related procedures) via a suprachoroidal approach, as described above with reference to FIG. 3. More particularly, aspects of the delivery device 2700 facilitate improved ergonomics for a user during suprachoroidal subretinal injections, as the delivery device 2700 may be held horizontally instead of vertically during performance of such injections.

As shown in FIG. 27A, the delivery device 2700 includes a handle 2702 for holding by a user. In certain embodiments, a flexible fluidic tubing 2720 for supplying an injection fluid to the delivery device 2700 may be disposed through a proximal end 2706 of the handle 2702 and fluidically coupled to a tubular injection cannula 2710 within the handle 2702. In certain embodiments, the fluidic tubing 2720 may couple to the proximal end 2706 of the handle 2702, or another fluidic tubing within the handle 2602 (described elsewhere herein). In certain other embodiments, the handle 2702 may comprise an actuatable internal chamber (not shown in FIG. 27A) fluidically coupled to the injection cannula 2710 and containing the injection fluid. In such embodiments, subretinal delivery device 2700 may not be coupled to any external fluidic tubing. In further embodiments, to simplify fluidic preparation for subretinal injection and/or the injection itself, a therapeutic agent or other injection fluid may be provided to the delivery device 2700 from a prefilled cartridge that can be coupled to a fluidic drive system within the delivery device 2700, or to an external fluidic system connected to the delivery device 2700 via the fluidic tubing 2720. Cartridges for therapeutic agents are described in more detail elsewhere herein.

A proximal end 2776 of a curved shaft adapter 2770 couples to and extends from a distal end 2704 of the handle 2702. The curved shaft adapter 2770 enables a user to hold the delivery device 2700 horizontally, instead of vertically, during performance of a subretinal injection or other procedure, thereby facilitating improved stability, control, and overall safety when using the delivery device 2700. Even further, the delivery device 2700, when held horizontally by the user, does not interfere with the optics of any visualization systems utilized, such as a microscope. Accordingly, the curved shaft adapter 2770 facilitates improved ergonomics for the user when using the delivery device 2700. In certain embodiments, the curved shaft adapter 2770 comprises a curved, curled, or bent hollow tube.

The shaft adapter 2770 may be defined by any suitable curvature for performing a subretinal injection via a suprachoroidal approach. For example, in certain embodiments, the shaft adapter 2770 has a radius R of curvature between 1 mm (millimeters) and about 20 mm, such as between about 5 mm and about 15 mm, such as about 10 mm. In certain embodiments, due to the curvature of the shaft adapter 2770, a distal end 2774 of the shaft adapter 2770 has a major axis S disposed at an angle between 0 degrees and 90 degrees relative to a major longitudinal axis A of the handle 2702. For example, in certain embodiments, the major axis S (and the major longitudinal axis of the cannula 2710) is disposed at an angle between 30 degrees and 60 degrees relative to the major axis A, such as an angle of 45 degrees relative to the major axis A. In certain embodiments, the major axis S (and the major longitudinal axis of the cannula 2710) is disposed at an angle between 45 degrees and 90 degrees relative to the major axis A, such as at an angle between about 60 degrees and 75 degrees relative to the major axis A. Generally, the curvature of the shaft adapter 2770 is such that any tubing and/or fluidics within the shaft adapter 2770 is not negatively affected by the curvature (e.g., the curvature doesn't cause kinking or sliding thereof), and such that the distance from a proximal end 2706 of the handle 2702 to the distal end 2774 of the shaft adapter 2770 is not too long for ergonomic use.

In certain embodiments, the curved shaft adapter 2770 may be formed of a rigid material, such as rigid metallic or polymeric material. Examples of rigid metallic materials include stainless steel, aluminum, and titanium.

A proximal end 2716 of an extendable injection cannula 2710 is coupled to the distal end 2774 of the shaft adapter 2770. The extendable injection cannula 2710 is configured to slidably extend from and retract into the distal end 2774 of the shaft adapter 2770, which allows the injection cannula 2710 to be extended through the suprachoroidal space, to a target injection site, after the injection cannula 2710 is inserted into the patient's eye. Such actuation of the injection cannula 2710 may be controlled by any suitable control mechanism. In FIG. 27A, actuation of the injection cannula 2710 is controlled by a toggle 2740 on the handle 2702. In certain embodiments, the toggle 2740 comprises a sliding button or switch, wherein sliding of the toggle 2740 by a user in a distal direction 2742 causes the injection cannula 2710 to extend from shaft adapter 2770, and sliding of the toggle 2740 in a proximal direction 2744 causes the injection cannula 2710 to retract into the shaft adapter 2770.

The injection cannula 2710 further comprises a distal tip 2711 disposed at a distal end 2714 thereof. In certain embodiments, the distal tip 2711 may have a tapered or sloped (e.g., ramp-like) profile in order to facilitate movement through the suprachoroidal space. In certain embodiments, the distal tip 2711 may have oval-like, widened, or flattened cross-section, to facilitate easier translation through the suprachoroidal space. Exemplary distal tips are discussed in more detail elsewhere herein. The injection cannula 2710 and/or distal tip 2711 are generally formed of any suitable flexible surgical-grade materials, such a flexible metallic or thermoplastic polymeric materials. Examples of flexible metallic materials include nitinol and other metallic alloys. Examples of suitable thermoplastic polymeric materials include polyimide. In certain embodiments, the distal tip 2711 is formed of a rigid material, and the remainder of the injection cannula 2710 is formed of a flexible material.

Turning now to FIG. 27B, a curved or straight injection needle 2712 is coupled to the distal tip 2711. In certain embodiments, the injection needle 2712 is configured to slidably extend from and retract into the distal tip 2711, which facilitates the prevention of damage to the injection needle 2712 and/or patient's eye during movement of the injection cannula 2710 through the suprachoroidal space. Such extension/retraction of the injection needle 2712 may be controlled by any suitable control mechanism, such as a toggle on the handle 2702 separate from toggle 2740. In exemplary embodiments, the injection cannula 2710 is a 23-, 25-, or 27-gauge needle, while the injection needle 2712 is a finer gauge needle, such as a 38-gauge needle. However, other sizes/gauges of injection cannulas and injection needles may be used in other embodiments.

In certain embodiments, as described elsewhere herein, the injection needle 2712 is formed of a flexible material, such as nitinol or polyimide. In certain embodiments, the injection needle 2712 is formed of a stiff material, including metallic materials such as stainless steel or thermoplastic polymers such as polyether ether ketone (PEEK), polyetherketone (PEK), and polytetrafluoroethylene (PTFE).

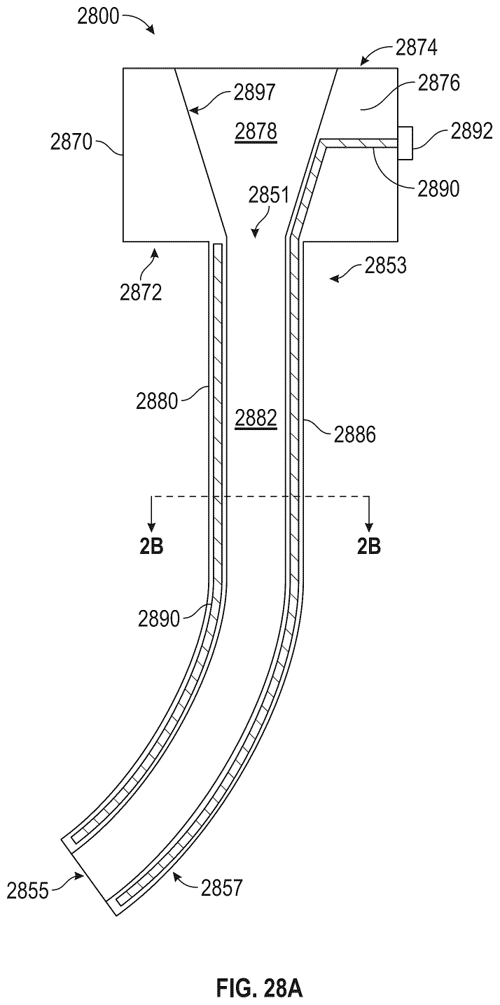

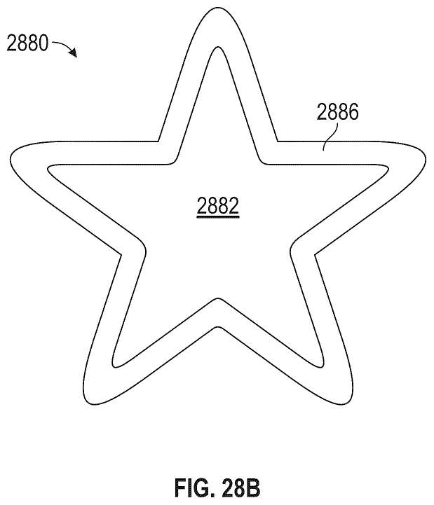

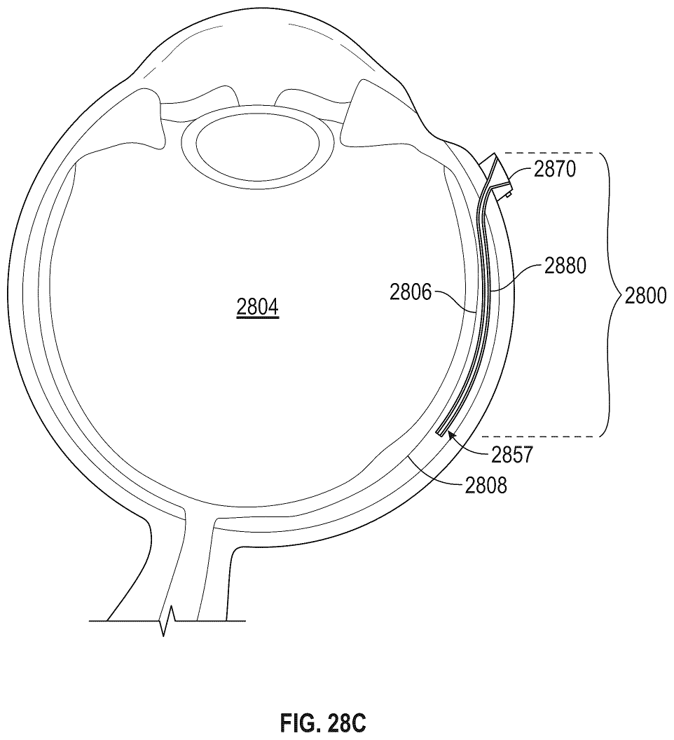

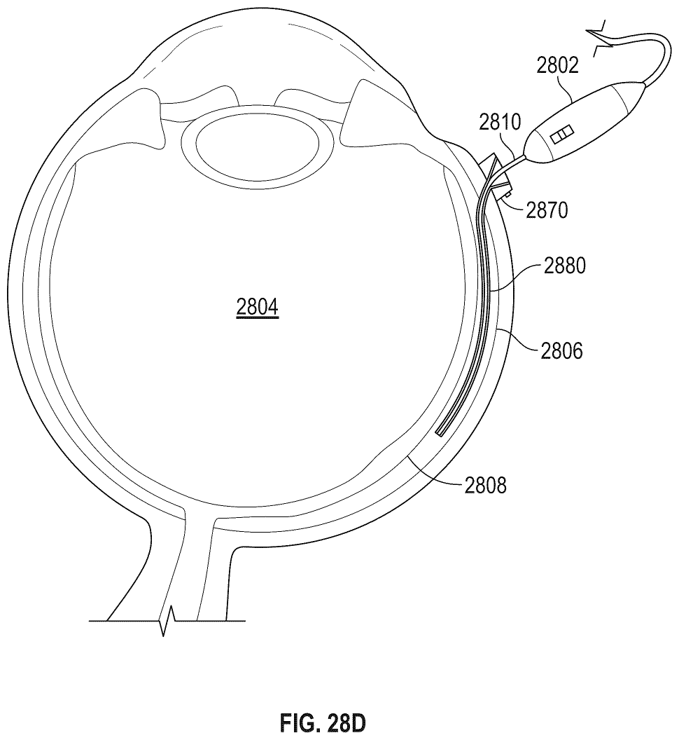

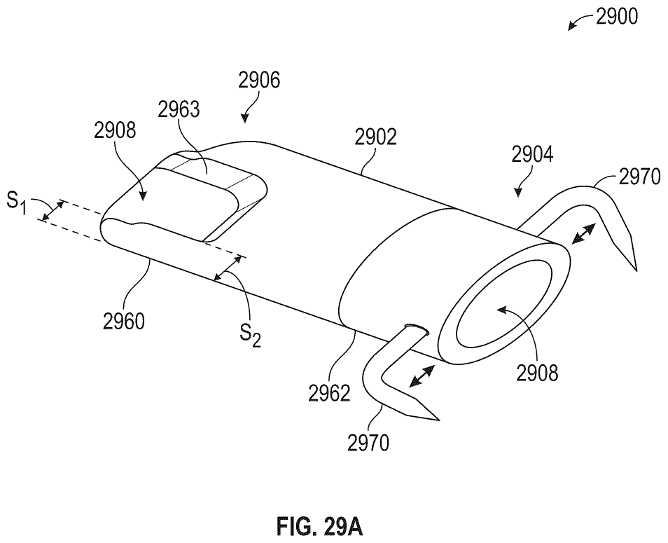

FIGS. 28A-28D illustrate various views of an exemplary guidance cannula 2800, according to certain embodiments of the present disclosure. The guidance cannula 2800 may be used in combination with other delivery devices described herein, e.g., delivery devices 1800 and 1801 of FIGS. 18A-18B. Thus, aspects of the guidance cannula 2800 may be combined with other delivery devices and/or components described herein without limitation.

In general, the guidance cannula 2800 comprises an expandable and flexible cannula that is configured to provide a predefined channel through which an injection cannula of a delivery device may be inserted for guided translation through the suprachoroidal space to a target injection site. For example, the guidance cannula may be first inserted into the patient's eye and advanced through the suprachoroidal space until a distal end thereof is positioned adjacent a target injection site. Thereafter, the guidance cannula may be expanded, and the injection cannula may be inserted into and advanced through the expanded guidance cannula until a distal end of the injection cannula reaches the target injection site (e.g., until the distal end of the injection cannula passes the distal end of the guidance cannula). The guidance cannula thus facilitates easier handling and positioning of the injection cannula during entry and positioning thereof within the suprachoroidal space. Furthermore, because the guidance cannula does not need to include an injection needle or any fluidics, the guidance cannula may have smaller lateral dimensions as compared to the injection cannula of the delivery device. Accordingly, the guidance cannula may impart less strain on the choroid during insertion thereof, which may reduce the overall damage thereto during a suprachoroidal subretinal injection.

Turning now to FIG. 28A, a cross-sectional side view of the guidance cannula 2800 is depicted. As shown, the guidance cannula 2800 includes a hub 2870 and a tube 2880. The tube 2880 couples to a bottom (e.g., distal) surface 2872 of the hub 2870 and extends distally therefrom. The tube 2880 is configured to be inserted into and passed through the suprachoroidal space of a patient's eye, and is further configured to facilitate the insertion and advancement of an injection cannula within. Accordingly, the tube 2880 may be generally tubular, with a circular, elliptical, or pill-shaped cross-sectional top profile (e.g., as viewed along the longitudinal length of the tube 2880) in certain embodiments. However, other top cross-sectional morphologies are also contemplated for the tube 2880, as described below. Further, the tube 2880 comprises a centrally disposed guidance channel 2882 that extends from an opening 2851 at a proximal end 2853 thereof to an opening 2855 at a distal end 2857. The guidance channel 2882 and openings 2851 and 2855 enable an injection cannula to pass through both ends of the tube 2880.

Meanwhile, the hub 2870 may, in certain embodiments, be substantially cylindrical or ring-like, though other morphologies are contemplated as well. The hub 2870 comprises a central channel 2878, which is fluidly coupled to the opening 2851 of the guidance channel 2882 and is further surrounded by, and partially defined by, an internal wall 2897 of the hub 2870. In certain embodiments, the hub 2870 may act as a stop or retention device, preventing the tube 2880 from entering too far into the suprachoroidal space during insertion. Accordingly, the bottom surface 2872 of the hub 2870 may be configured to be flush with a surface of a patient's eye, and the hub 2870 may have a larger outer diameter (or other lateral dimension) larger than that of the tube 2880. Additionally, the hub 2870 may act as an adapter to facilitate easier insertion of an injection cannula into the tube 2880. Thus, a top (e.g., proximal) surface 2874 and/or the inner wall 2897 may have a sloped, ramp-like, and/or conical shape to mechanically guide the injection cannula into the guidance channel 2882 of the tube 2880.

In certain embodiments, the hub 2870 and the tube 2880 are monolithically formed such they comprise the same material. For example, both of the hub 2870 and the tube 2880 may be formed of a flexible and expandable material, such as silicone, polyurethane (PUR), polyether block amide (PEBA), polyolefin, combinations thereof, and the like. In certain other embodiments, the hub 2870 and the tube 2880 may comprise different materials. For example, the hub 2870 may be formed of a stiff or non-expandable material, such as metallic material like stainless steel, aluminum, titanium, or other metallic alloy, or the hub 2870 may be formed of a thermoplastic polymer such as polyether ether ketone (PEEK), polyetherketone (PEK), and polytetrafluoroethylene (PTFE); meanwhile, the tube 2880 may be formed of a flexible/elastic and expandable material such as plastic, metal, polymer, nitinol, or combinations thereof.

As further shown in FIG. 28A, in certain embodiments, the hub 2870 and/or the tube 2880 comprise an expansion channel 2890. The expansion channel 2890 may be disposed within a wall 2876 of the hub 2870 and/or a wall 2886 of the tube 2880. In certain embodiments, the expansion channel 2890 circumscribes at least a portion of the guidance channel 2882 formed centrally within the tube 2880. In certain embodiments, the expansion channel 2890 circumscribes an entire, or nearly entire, longitudinal length of the guidance channel 2882 formed centrally within the tube 2880. In certain embodiments, the channel 2890 is fluidically coupled to closable port 2892 formed in the hub 2870 and/or the tube 2880. The closable port 2892 enables ingress and egress into the channel 2890 from an exterior of the guidance cannula 2800. Accordingly, the channel 2890 can be filled with fluids to cause the tube 2880 (an in certain embodiments, the hub 28170) to expand, thereby increasing the lateral dimensions (e.g., diameter) of at least the tube 2880 and, as a result, the lateral dimensions (e.g., diameter) of the guidance channel 2882 to facilitate the entry and passage of an injection cannula of a delivery device through the tube 2880. In certain embodiments, the channel 2890 may be filled with a gas, such as air, oxygen, nitrogen (N2), or other gases. In certain embodiments, the channel 2890 may be filled with a liquid, such as perfluorocarbon liquid (PFCL), BSS, saline or other liquids. In certain embodiments, the channel 2890 may be filled with a combination of liquid and gas.

In certain embodiments, however, the tube 2880 may be expanded by other means, including mechanical means. For example, in certain embodiments, the tube 2880 may expand utilizing the intrinsic spring-like action of a braided wire when the braided wire is twisted by a user. The braided wire may be disposed in the expansion channel 2890 within the wall 2886 of the tube 2880, or the braided wire may be disposed within and circumferentially lining the guidance channel 2882. Yet, other mechanical means for expansion of the tube 2880 are further contemplated.

In certain embodiments, the guidance cannula 2800 may not comprise any separate expansion mechanisms or devices, other than being formed of a flexible material. For example, in such embodiments, the tube 2880 may be latently expanded by the insertion of an injection cannula therethrough, which may have an outer diameter greater than the lateral dimensions (e.g., diameter) of the guidance channel 2882. Thus, the tube 2880 may be expanded as the injection cannula advances through the tube 2880.

In certain embodiments, an expandability of the tube 2880 may be optimized based on a cross-sectional profile thereof. For example, in certain embodiments, the tube 2880 may have a star-shaped cross-sectional profile, or other suitably shaped profile, such that expansion of the tube 2880 is caused by “unfolding” of the wall 2886 rather than, or in addition to, stretching of the wall 2886. A cross-sectional top view of an exemplary star-shaped cross-sectional profile of the tube 2880 is depicted in FIG. 28B for reference. The unfolding of the wall 2886 may decrease the stress on the tube 2880 during expansion and/or decrease the amount of fluid pressure or force needed to expand the tube 2880, thereby facilitating easier and more reliable expansion of the tube 2880 during use.

FIGS. 28C and 28D illustrate schematic cross-sectional side views of an exemplary guidance cannula 2800 during use. In FIG. 28C, the guidance cannula 2800 is first inserted into the suprachoroidal space 2806 of a patient's eye 2804 and advanced through the suprachoroidal space 2806 until a distal end 2857 of the tube 2880 is positioned adjacent a target injection site 2808. Thereafter, in FIG. 28D, the guidance cannula 2800 may be expanded, such as by flowing fluids into the expansion channel 2890 (and sealing the closable port 2892). An injection cannula 2810 of a delivery device 2802 may then be inserted into and advanced through the expanded guidance cannula 2800, and more particularly, through the tube 2880, until a distal end of the injection cannula 2810 passes the distal end 2857 adjacent to the target injection site 2808. At this point, an injection needle of the injection cannula 2810 may be extended for piercing the choroid to inject fluids into the subretinal space.

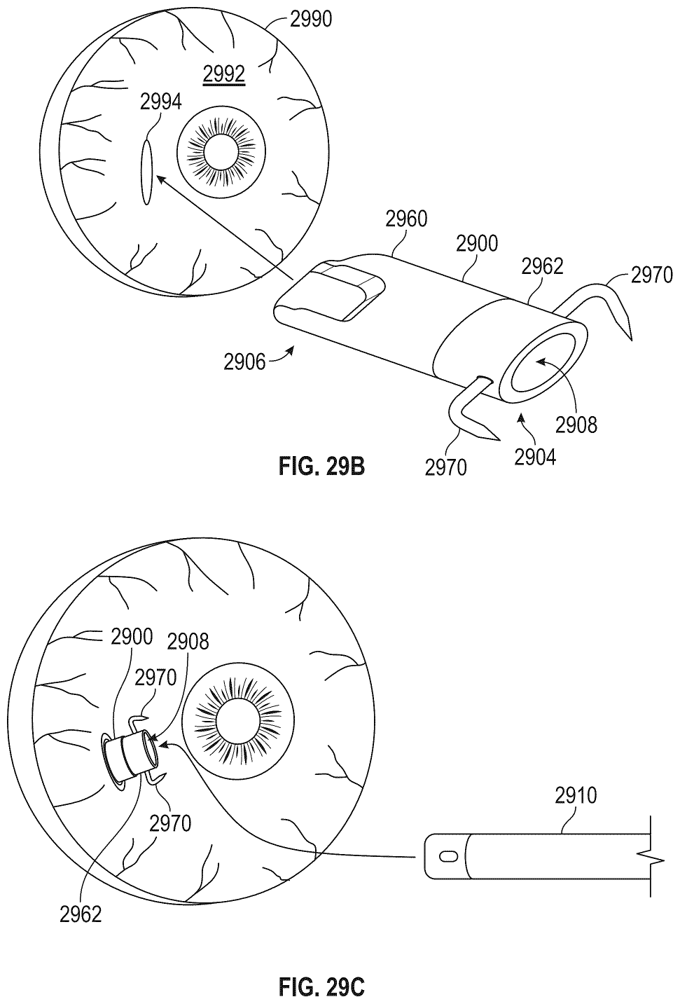

FIGS. 29A-29C illustrate various views of an exemplary entry cannula 2900, according to certain embodiments of the present disclosure. The entry cannula 2900 is an exemplary entry cannula that may be utilized to facilitate entry of the injection cannula of a delivery device through the sclera of the eye and into the suprachoroidal space, as described above with reference to FIG. 3. Accordingly, the entry cannula 2900 may be utilized in combination with, e.g., the delivery devices 1800 and 1801 of FIGS. 18A-18B, delivery device 2600 of FIG. 26, and other delivery devices for subretinal injection as described herein. However, aspects of the entry cannula 2900 may be combined with other delivery devices and/or components described herein without limitation.

Turning now to FIG. 29A, the entry cannula 2900 comprises a tubular body 2902 having a central channel 2908 extending from a proximal end 2904 of the body 2902 to a distal end 2906 of the body 2902. After insertion of the entry cannula 2900 through the sclera of a patient's eye, the central channel 2908 functions as an entry point or port for the subsequent insertion of an injection cannula of a delivery device. Accordingly, the central channel 2908 may have lateral dimensions (e.g., a diameter) along a longitudinal length thereof that are substantially the same or greater than those of the injection cannula to be inserted therethrough. In certain embodiments, the central channel 2908 has uniform dimensions from the proximal end 2904 to the distal end 2906. In certain embodiments, the central channel 2908 has non-uniform lateral dimensions from the proximal end 2904 to the distal end 2906; for example, the central channel 2908 may have greater lateral dimensions at or near the proximal end 2904 as compared to the distal end 2906.

In certain embodiments, the body 2902 may have a generally circular cross-sectional profile. In certain other embodiments, as shown in FIG. 29A, the body 2902 may have a “flattened” cross-sectional profile, which may resemble an elliptical or oval shape, or a pill or rounded-rectangular shape.

In certain embodiments, the body 2902 comprises a distal spatula portion 2960 including the distal end 2906 and a proximal entry portion 2962 including the proximal end 2904. Generally, the spatula portion 2960 may have ramp or wedge-like morphology, such that a vertical dimension S₁ of the spatula portion 2960 at the distal end 2906 gradually transitions proximally to a vertical dimension S₂ of the spatula portion 2960. The ramp or wedge-like morphology facilitates delamination (i.e., separation) of the choroid as the distal end 2906 of the entry cannula 2900 is advanced into the suprachoroidal space after having passed through the sclera of the patient's eye. In certain embodiments, the spatula portion 2960 comprises a cutout 2963 formed in a side wall of the body 2902 that fluidly couples with the central channel 2908 and improves efficiency of delamination.

In certain embodiments, the spatula portion 2960 is formed of a stiff material, including metallic materials such as aluminum, stainless steel, and other metallic alloys, or thermoplastic polymeric materials such as polyether ether ketone (PEEK), polyetherketone (PEK), and polytetrafluoroethylene (PTFE). In certain other embodiments, the spatula portion 2960 is formed of a flexible material, including metallic materials such as nitinol and thermoplastic polymeric materials such as polyimide. Utilization of a flexible material for the spatula portion 2960 may reduce strain on the choroid, retina, and/or sclera during insertion of the entry cannula 2900, thereby reducing choroidal, retinal, and/or scleral damage caused by the entry cannula 2900 during use.

The entry portion 2962, meanwhile, may be tube-like and generally configured to facilitate the entry and advancement of an injection cannula into the entry cannula 2900. In certain embodiments, the entry portion 2962 is formed of a stiff material, including metallic materials such as aluminum, stainless steel, and other metallic alloys, or thermoplastic polymeric materials such as polyether ether ketone (PEEK), polyetherketone (PEK), and polytetrafluoroethylene (PTFE). In certain other embodiments, the entry portion 2962 is formed of a flexible material, including metallic materials such as nitinol and thermoplastic polymeric materials such as polyimide. In certain embodiments, the entry portion 2962 and the spatula portion 2960 may be formed of the same material and thus, the body 2902 may comprise a single, monolithic component. In certain other embodiments, however, the entry portion 2962 and the spatula portion 2960 may be formed of different materials.

In certain embodiments, the entry portion 2962 optionally comprises one or more fixation arms 2970 coupled thereto (two fixation arms 2970 are shown in FIG. 29A, extending laterally from opposing sides of the entry portion 2962). The one or more fixation arms 2970 may be configured to function as a “stop” to immobilize the entry cannula 2900 in place after the entry cannula 2900 has been inserted through the sclera and advanced to a desired depth in the suprachoroidal space. Accordingly, once the entry cannula 2900 has been advanced to the desired depth, the fixation arms 2970 may be configured to contact an outer surface of the sclera disposed outward of the incision through which the spatula portion 2960 and a portion of the entry portion 2962 are inserted, thereby preventing the entry cannula 2900 from progressing any further into the patient's eye. The one or more fixation arms 2970 may thus prevent any unintended movement of the entry cannula 2900 after final positioning thereof, thereby reducing the risk of damage to the choroid, retina, and/or sclera and facilitating improved control of injection cannula positioning during a subretinal injection. In certain embodiments, the one or more fixation arms 2970 may be fixedly coupled to the entry portion 2962. In certain other embodiments, the one or more fixation arms 2970 may be extendably coupled to the entry portion 2962, thereby enabling the one or more fixation arms 2970 to be actively extended by the user laterally outward from the entry portion 2962, or perpendicular to a major longitudinal axis of the entry cannula 2900, during use. Generally, the fixation arms 2970 may have any suitable dimensions and morphologies. In the example of FIG. 29A, the fixation arms 2970 are depicted as curved or bent wires resembling “horns” that may be extended laterally from the entry portion 2962.

FIGS. 29B and 29C illustrate perspective views of the entry cannula 2900 during use. In FIG. 29B, the entry cannula 2900 is first inserted through an incision 2994 in the sclera 2992 of a patient's eye 2990. In embodiments comprising fixation arm(s) 2970, the entry cannula 2900 is inserted through the incision until the one or more fixation arms 2970 contact the sclera 2992. In certain embodiments, the sclera 2992 may be incised utilizing a trocar in combination with the entry cannula 2900. For example, a trocar may be disposed through and extending from a distal end of the central channel 2908 of the entry cannula 2900. The portion of the trocar extending from the central channel 2908 is then inserted into the eye 2990, thereby forming the incision, until bottom surface(s) of the one or more fixation arms 2970 contact the sclera 2992. Then, the trocar may be removed from the eye 2990, leaving the entry cannula 2900 in place.

Thereafter, in FIG. 29C, an injection cannula 2910 of a delivery device may be inserted into and advanced through the entry cannula 2900 until a distal end of the injection cannula 2910 is disposed adjacent a target subretinal injection site for injection. The utilization of the entry cannula 2900 enables the user to view the advancement of the injection cannula 2910 through the suprachoroidal space via a microscope, without the user needing to focus on the sliding of the injection cannula 2910 through the sclerotomy. This ultimately facilitates better control and more efficient placement of the injection cannula 2910, while also reducing the risk of damage to ophthalmic tissues.

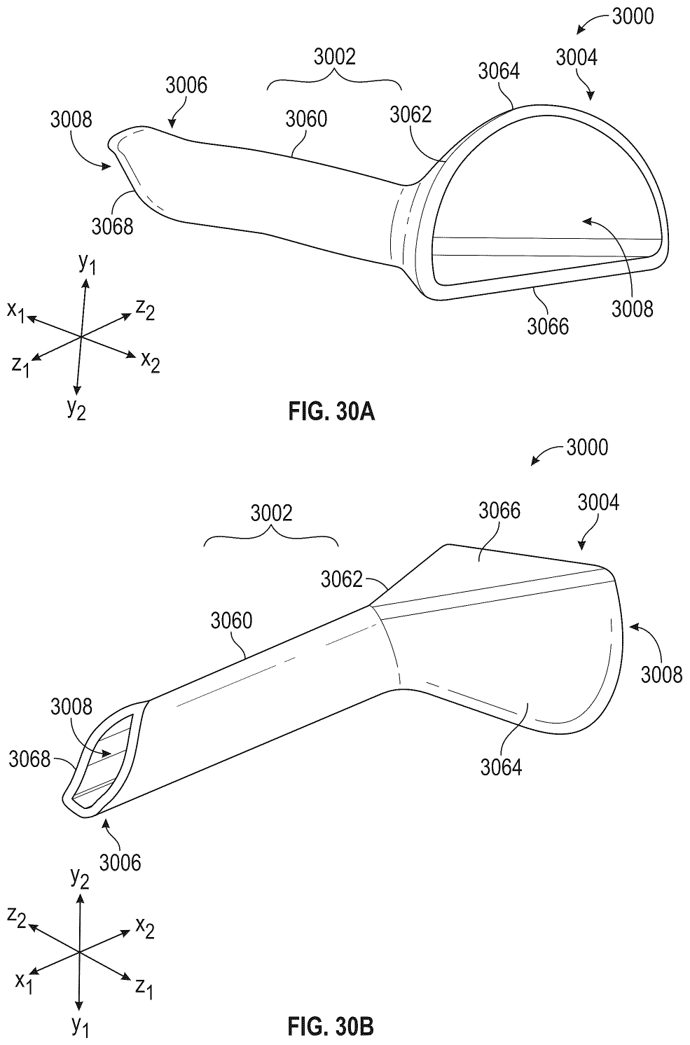

FIGS. 30A and 30B illustrate perspective views of another exemplary entry cannula 3000, according to certain embodiments of the present disclosure. Similar to the entry cannula 2900, the entry cannula 3000 is an exemplary entry cannula that may be utilized to facilitate entry of the injection cannula of a delivery device through the sclera of the eye and into the suprachoroidal space, as described above with reference to FIG. 3. Accordingly, the entry cannula 3000 may be utilized in combination with, e.g., the delivery devices 1800 and 1801 of FIGS. 18A-18B, delivery device 2600 of FIG. 26, and other delivery devices for subretinal injection as described herein. However, aspects of the entry cannula 3000 may be combined with other delivery devices and/or components described herein without limitation.

As shown, the entry cannula 3000 comprises a tubular body 3002 having a central channel 3008 extending from a proximal end 3004 of the body 3002 to a distal end 3006 of the body 3002. After insertion of the entry cannula 3000 through the sclera of a patient's eye, the central channel 3008 functions as an entry point or port for the subsequent insertion of an injection cannula of a delivery device. Accordingly, the central channel 3008 may have lateral dimensions along a longitudinal length thereof that are substantially the same or greater than those of the injection cannula to be inserted therethrough.

In certain embodiments, the body 3002 comprises a distal tube portion 3060 including the distal end 3006 and a proximal funnel portion 3062 including the proximal end 3004. The distal tube portion 3060 is generally tubular, and may have a top cross-section resembling a circle or oval-like shape. As shown in FIGS. 30A and 30B, an endface 3068 of the tube portion 3060 may be disposed at a non-normal angle relative to a major longitudinal axis of the tube portion 3060, thereby forming a ramp or wedge-like morphology at the distal end 3006. This ramp or wedge-like morphology facilitates delamination (i.e., separation) of the choroid as the distal end 3006 of the entry cannula 3000 is advanced into the suprachoroidal space after having passed through the sclera of the patient's eye. In certain embodiments, the tube portion 3060 is formed of a stiff material, including metallic materials such as aluminum, stainless steel, and other metallic alloys, or thermoplastic polymeric materials such as polyether ether ketone (PEEK), polyetherketone (PEK), and polytetrafluoroethylene (PTFE). In certain other embodiments, the tube portion 3060 is formed of a flexible material, including metallic materials such as nitinol and thermoplastic polymeric materials such as polyimide. Utilization of a flexible material for the tube portion 3060 may reduce strain on the choroid, retina, and/or sclera during insertion of the entry cannula 3000, thereby reducing choroidal, retinal, and/or scleral damage caused by the entry cannula 3000 during use.

The funnel portion 3062, meanwhile, may be generally configured and shaped to facilitate the entry and advancement of an injection cannula into the entry cannula 3000. In certain embodiments, the funnel portion 3062 comprises a funnel-like or substantially funnel-like morphology. The funnel-like, or substantially funnel-like, morphology of the funnel portion 3062 facilitates the mechanical guidance of an injection cannula into the central channel 3008 during use. For example, in the embodiments of FIGS. 30A and 30B, the funnel portion 3062 comprises a semi-funnel morphology including a hyperbolic wall 3064 coupled to a planar wall 3066. In such embodiments, the hyperbolic wall 3064 may mechanical direct an injection cannula into the central channel 3008, as the hyperbolic wall 3064 conically tapers proximally toward the central channel 3008. Simultaneously, the planar wall 3066 may facilitate the positioning of the funnel portion 3062 against the outer surface of the sclera of the patient's eye. In other words, the planar wall 3066 is configured to lay flat against the outer surface of the eye, which improves the stability of entry cannula 3000 during use and reduces unwanted movement thereof. In certain embodiments, the planar wall 3066 may also indicate an orientation of the endface 3068 of the tube portion 3060, thereby facilitating easier positioning and orienting of the entry cannula 3000 after it has already been inserted through the sclera. For example, the planar wall 3066 may be disposed on the same or opposing side of the entry cannula 3000 as the endface 3068 is facing, and thus, a user may know the orientation of the endface 3068 by simply looking at the orientation of the planar wall 3066.

In certain embodiments, the funnel portion 3062 is formed of a stiff material, including metallic materials such as aluminum, stainless steel, and other metallic alloys, or thermoplastic polymeric materials such as polyether ether ketone (PEEK), polyetherketone (PEK), and polytetrafluoroethylene (PTFE). In certain other embodiments, the funnel portion 3062 is formed of a flexible material, including metallic materials such as nitinol and thermoplastic polymeric materials such as polyimide. In certain embodiments, the funnel portion 3062 and the tube portion 3060 may be formed of the same material and thus, the body 3002 may comprise a single, monolithic component. In certain other embodiments, however, the funnel portion 3062 and the tube portion 3060 may be formed of different materials.

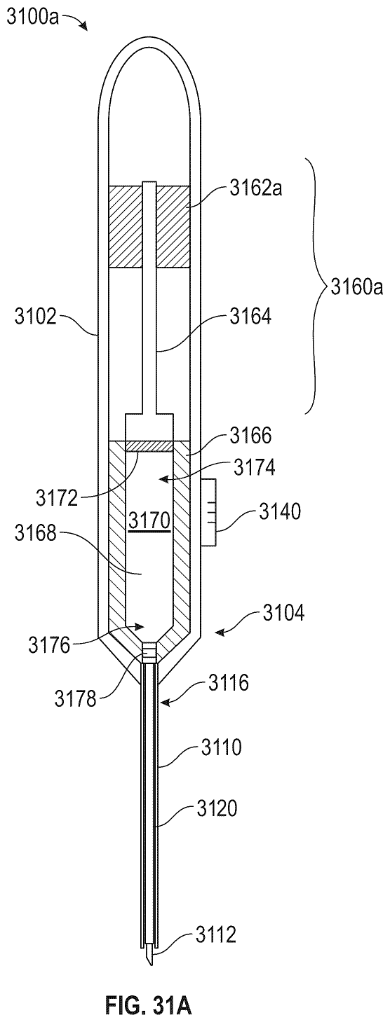

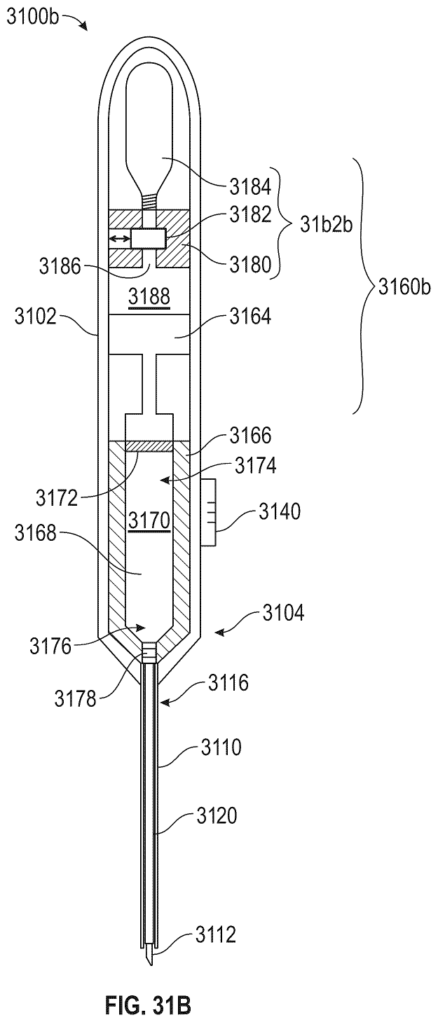

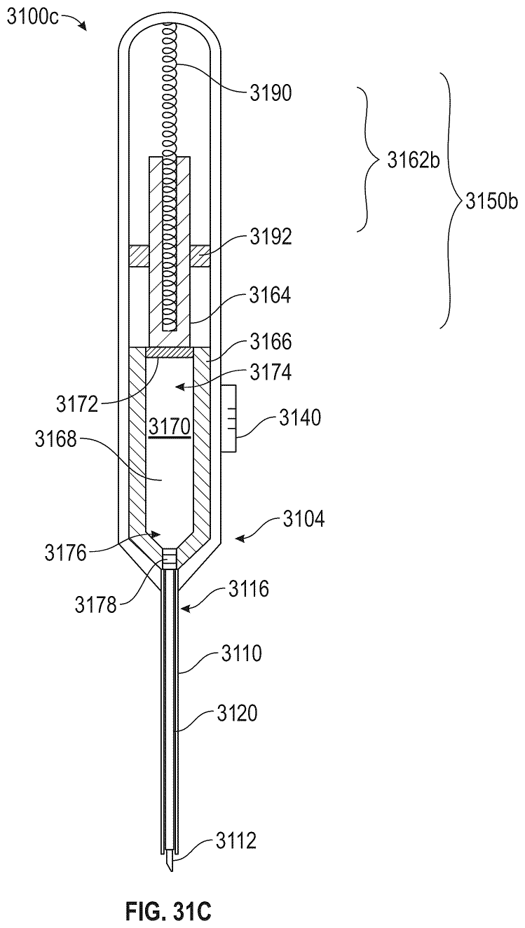

FIGS. 31A-31C illustrate schematic cross-sectional views of exemplary subretinal delivery devices 3100a, 3100b, and 3100c, respectively, according to certain embodiments of the present disclosure. The delivery devices 3100a-c comprise handles with fluidic drive systems integrated therein, which facilitates easier handling of the delivery device 3100a-c during subretinal injection procedures and reduces the number of components necessary for such procedures. The delivery devices 3100a-c may be utilized as, e.g., the delivery device 414 of the surgical system 400 of FIG. 4, and aspects thereof may be combined with other delivery devices and/or components described herein without limitation.

Turning now to FIG. 31A, the delivery device 3100a includes a handle 3102 and an injection cannula 3110 having a proximal end 3116 coupled to and extending distally from a distal end 3104 of the handle 3102. The injection cannula 3110 may comprise any suitable type of injection cannula, including those described elsewhere herein. A curved or substantial straight injection needle 3112 is disposed within the injection cannula 3110 for piercing a desired ocular tissue (e.g., the retina or choroid) to deliver fluid to the subretinal space. Similar to the injecting cannula 3110, the injection needle 3112 may comprise any suitable type of injection needle, including those described elsewhere herein. In certain embodiments, the injection needle 3112 is configured to slidably extend from and retract into a distal end 3114 of the injection cannula 3110, via manual control of a toggle 3140 of the handle 3102. In certain embodiments, the injection needle 3112 is coupled to an inner fluidic shaft 3120 at least partially disposed within the cannula 3110. In such embodiments, the inner fluidic shaft 3120 may be slidably disposed within the cannula 3110 to facilitate extension and retraction of the injection needle 3112 upon actuation of the toggle 3140.

To simplify fluidic preparation and delivery for subretinal injection, a fluidic drive system 3160a is integrated within the handle 3102. The fluidic drive system 3160a simplifies subretinal injection procedures since an external fluidic drive system (e.g., an external fluid source and fluid pump) is no longer required to be connected to the delivery device 3100a. Accordingly, a surgeon can focus more of their attention to the procedure and the delivery device 3100a being used during the procedure, without concerning themselves with the setup and function of an external fluidic drive system. This, in turn, improves the efficiency and safety of the subretinal injection procedure. Even further, without an external fluidic drive system, the risk of fluidic leaks or other malfunctions during the subretinal injection procedure is greatly reduced.

In FIG. 31A, the fluidic drive system 3160a comprises an electromechanical and/or electromagnetic drive unit 3162a, one or more pistons 3164 operably coupled to the drive unit 3162a, and a cartridge 3166 configured to operably couple to the piston(s) 3164. The drive unit 3162a is configured to generate and impart a force or power onto the piston(s) 3164, which in turn causes the piston(s) 3164 to translate and act against the cartridge 3166 to dispense/deliver injection fluids 3168 contained therein.

The drive unit 3162a may generally comprise any suitable type of electromechanical and/or electromagnetic actuators for axially translating the one or more pistons 3164 within the handle 3102. For example, in certain embodiments, the drive unit 3162a comprises one or more electromechanical linear or rotary stepper motors. In certain embodiments, the drive unit 3162a comprises a rotary lead screw motor, and the piston(s) 3164 comprise a threaded slider configured to mate with the rotary lead screw. Upon the drive unit 3162a receiving a control signal, the drive unit 3162a generates mechanical rotary or linear force on the one or more pistons 3164 operably coupled therewith to axially translate the piston(s) 3164 within the handle 3102. In certain embodiments, the control signal is provided from a foot controller (e.g., foot pedal 410), surgical console (e.g., surgical console 402), or other component of a surgical system within an operating environment in wired or wireless communication with the delivery device 3100a. To facilitate wireless control of the drive unit 3162a, the delivery device 3100a may further comprise a wireless communication module 3150, which may include a wireless transmitter and receiver circuitry to relay signals (e.g., instructions) to and from the delivery device 3100a and particularly, the drive unit 3162a. In certain embodiments, the wireless communication module 3150 may be in wireless communication with a foot controller or surgical console to enable remote control of the drive unit 3162a.

The cartridge 3166 comprises any suitable fluid cartridge having one or more lumens 3170 at least partially defining a volume (e.g., reservoir) for storing an injection fluid 3168. In certain embodiments, the cartridge 3166 comprises an interchangeable and disposable cartridge that has been prefilled with injection fluids 3168 prior to insertion into the handle 1802. In such embodiments, the cartridge 3166 may comprise a single lumen 3170 prefilled with a premixed solution of both treatment solution and non-treatment solution, as described above with reference to FIG. 16D. For example, the single lumen 3170 may comprise the premixed solution having the desired concentrations/ratios of treatment solution (e.g., therapeutic agent) and non-treatment solution. In other examples, however, the cartridge 3166 may comprise two or more lumens 3170 prefilled with unmixed solutions of treatment solution and/or non-treatment solution. In such examples, the treatment solution and non-treatment solution may be mixed to desired concentrations/ratios within the cartridge 3166 after insertion of the cartridge 3166 into the handle and/or during injection.

The utilization of prefilled and interchangeable/disposable cartridges 3166 provides many advantages for surgeons when performing subretinal injections procedures with the delivery device 3100a. For example, because the cartridges 3166 are prefilled with all necessary injection fluids, no additional fluid preparation is necessary prior to performing the subretinal injection procedure, and accurate concentrations/ratios of the components of the delivered injection fluids is ensured. Further, such prefilled cartridges 3166 enable the surgeon to decide on short notice which therapeutic substances to use for the subretinal injection based on the current circumstances of the patient. Additionally, because therapeutic substances typically have a shorter shelf life than delivery devices, the separation of the cartridge 3166 from the delivery device 3100a enables the surgeon to store the delivery device 3100a for longer periods of time, without worrying about having to dispose the delivery device 3100a due to the expiration of a therapeutic substance.

In still other embodiments, however, the cartridge 3166 comprises a container fixedly integrated with the handle 3102. In such embodiments, the cartridge 3166 and handle 3102 may comprise one or more ports for filling the cartridge 3166 with the treatment solution and non-treatment solution prior to an injection.

As further shown in FIG. 31A, in certain embodiments, a movable seal or stopper 3172 is disposed at a proximal end 3174 of each lumen 3170 of the cartridge 3166 and operably coupled to one of the one or more pistons 3164. Meanwhile, the cartridge 3166 comprises a valved port 3178 at a distal end 3176 of each lumen 3170 that is configured to be in fluid communication with the injection cannula 3110, injection needle 3112, and/or inner fluidic shaft 3120. During use, distal axial translation of the piston(s) 3164, as driven by the drive unit 3162a, will cause the piston(s) 3164 to mechanically engage and push the seal(s) 3172 distally through the lumen(s) 3170, thereby forcing the injection fluids 3168 to dispense through the valved port 3178 into the cannula 3110 (and/or inner fluidic shaft 3120) and injection needle 3112. In certain embodiments, a force of the injection fluids 3168 against the valved port 3178 causes the valved port 3178 to open to facilitate flow of the injection fluids 3168 therethrough. In certain embodiments, engagement of the cartridge 3166 with the handle 3102 upon insertion creates a puncture or opening in the valved port 3178 to facilitate flow of the injection fluids 3168 therethrough.

During operation of the delivery device 3100a, the user may activate and control the drive unit 3162a by operation of a foot controller, thus controlling movement of the piston(s) 3164. For example, the user may depress the foot pedal 410 described in FIG. 4 to activate the drive unit 3162a and axially translate the piston(s) 3164 in a forward (e.g., distal) “injection” movement, thereby forcing the injection fluids 3168 out of the cartridge 3166. In certain embodiments, the injection rate (e.g., output flow rate) of injection fluids 3168 is predetermined and controlled by drive unit 3162a. In certain embodiments, the user may increase the injection rate by further depressing the foot pedal 410 to increase the movement of the piston(s) 3164. Alternatively, reducing depression of the foot pedal 410 may slow the movement of the piston(s) 3164 in the injection direction, thereby reducing the injection rate. Applying no pressure to the foot pedal 410 may cause the foot pedal 410 to transition into a fully undepressed state and, thereby, completely stop the movement of the piston(s) 3164 altogether, and in turn, stop injection. In certain embodiments, the movement speed of the piston(s) 3164, and thus, the injection rate, may linearly correspond to the position of the foot pedal 410.

In certain embodiments, the user may also control the piston(s) 3164 to move in a reverse (e.g., proximal) direction, thus enabling the delivery device 3100a to draw up fluid into the injection needle 3112 and cannula 3110 (and/or inner fluidic shaft 3120). For example, the user may depress a switch on the foot pedal 410 to activate a reverse mode of the delivery device 3100a, wherein subsequent depression of the foot pedal 410 causes actuation of the piston(s) 3164 in a proximal direction opposite the injection direction. The reverse mode may include the same mechanics as described above, wherein the reverse movement speed of the piston(s) 3164 linearly corresponds to the position of the foot pedal 410.

Turning now to FIG. 31B, the delivery device 3100b is substantially similar to delivery device 3100a, but for certain aspects of a fluidic drive system 3160b thereof. For purposes of clarity, only differentiating aspects will be described below.

Instead of the drive unit 3162a, the fluidic drive system 3160b comprises a drive unit 3162b. Similar to drive unit 3162a, the drive unit 3162b is configured to generate and impart a force or power onto piston(s) 3164, which in turn causes the piston(s) 3164 to translate and act against the cartridge 3166 to dispense/deliver injection fluids 3168 contained therein; however, unlike drive unit 3162a, the drive unit 3162b comprises an electro-pneumatic driver for axially translating the one or more pistons 3164 within the handle 3102. Thus, the drive unit 3162b may be described as an electro-pneumatic drive.

In the exemplary embodiment depicted in FIG. 31B, the drive unit 3162b comprises an electromotive actuator 3180, one or more fluid canisters 3184 storing a pressurized fluid, and a valve 3182 disposed over and sealing an opening 3186 of each fluid canister 3184 and operably connected to the electromotive actuator 3180. Examples of suitable pressurized fluids include but are not limited to carbon dioxide, nitrogen, and argon.

Upon the drive unit 3162b receiving a control signal during use of the delivery device 3100b, the electromotive actuator 3180 may open and/or close the valve(s) 3182 to control the flow rate of the pressured fluid through the opening(s) 3186 and into a pressurization pocket 3188 disposed between each fluid canister 3184 and a corresponding piston 3164. In a closed state, each valve 3182 prevents any flow of fluid into the corresponding pressurization pocket 3188. When the valve 3182 is opened, the pressurized fluid is allowed to flow into the pressurization pocket 3188 at a controlled flow rate depending on the position of the valve 3182. The accumulation of pressurized gas in the pressurization pocket 3188 applies a force to the proximal side of the corresponding piston 3164, thereby causing forward (e.g., distal) movement of the piston 3164 to dispense the injection fluids 3168 from the cartridge 3166. The valve(s) 3182 may comprise any suitable type of flow control valves operated by an electromechanical, electromagnetic or electro-pneumatic actuator 3180. Suitable valves include, but are not limited to, solenoid-type valves, proportional valves, plug valves, piston valves, knife valves, or the like.

Turning now to FIG. 31C, the delivery device 3100c is substantially similar to delivery devices 3100a and 3100b, but for certain aspects of a fluidic drive system 3160c thereof. For purposes of clarity, only differentiating aspects will be described below.

The fluidic drive system 3160c comprises a drive unit 3162c. Similar to the drive units above, the drive unit 3162c is configured to generate and impart a force or power onto piston(s) 3164, which in turn causes the piston(s) 3164 to translate and act against the cartridge 3166 to dispense/deliver injection fluids 3168 contained therein; however, in FIG. 31C, the drive unit 3162c comprises a spring-actuated mechanism for axially translating the one or more pistons 3164 within the handle 3102. Thus, the drive unit 3162c may be described as a spring-actuated drive.

In the exemplary embodiment depicted in FIG. 31C, the drive unit 3162c comprises a spring or similar device 3190 and a stopping mechanism or brake 3192 operably coupled to each of the one or more pistons 3164. Each spring 3190 is proximally disposed against the corresponding piston 3164 and provides a constant biasing force against the piston 3164 in the distal direction. Simultaneously, however, the stopping mechanism 3192 provides a stopping force against the piston 3164 to prevent translation of the piston 3164 as biased by the spring 3190. The stopping force may be provided against the piston 3164 as a friction force in a laterally inward direction normal to a major longitudinal axis of the handle 3102 (as in FIG. 31C), or the stopping force may be provided in a proximal direction against the piston 3164.

Upon the drive unit 3162c receiving a control signal during use of the delivery device 3100c, the stopping mechanism 3192 may controllably release the corresponding piston 3164, thereby allowing the spring 3190 to actuate the piston 3164 in the distal direction and act upon the cartridge 3166 to dispense the injection fluids 3168. In certain embodiments, the rate of injection may be controlled by inversely adjusting the amount of stopping force provided against the piston(s) 3164 by the stopping mechanism(s) 3192. For example, decreasing the amount of stopping force may increase the flow rate of the injection fluids 3168 from the cartridge 3166, while increasing the amount of stopping force may decrease the flow rate of the injection fluids 3168 from the cartridge 3166.

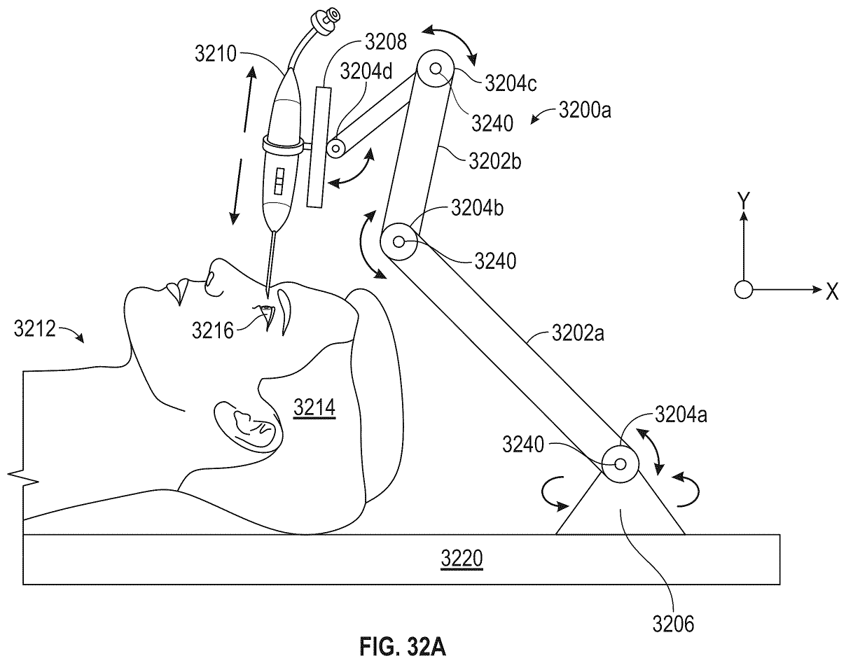

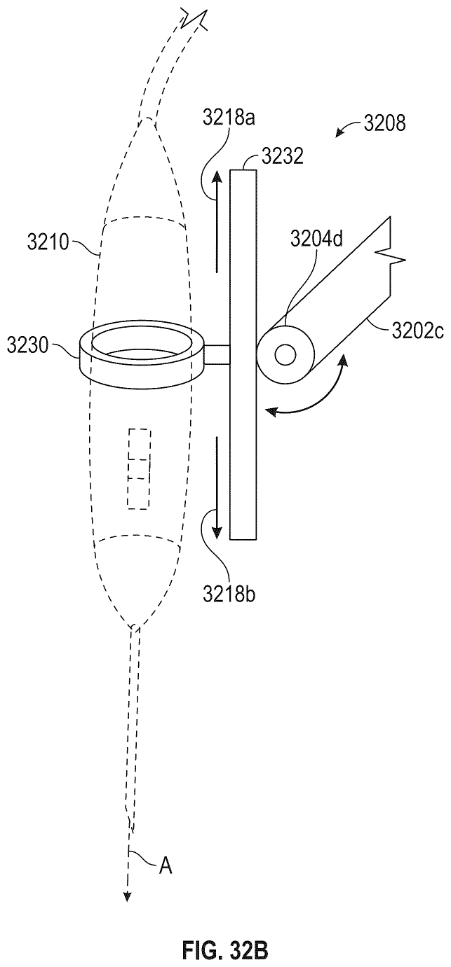

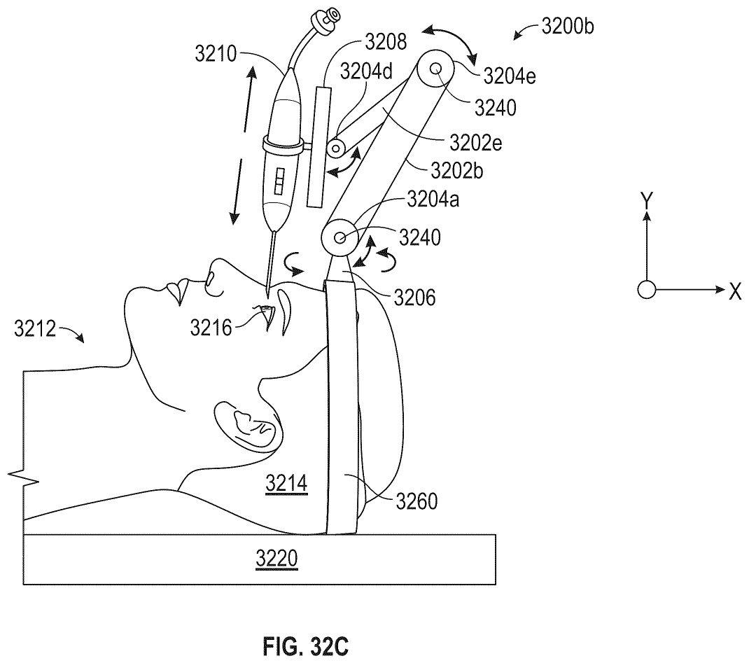

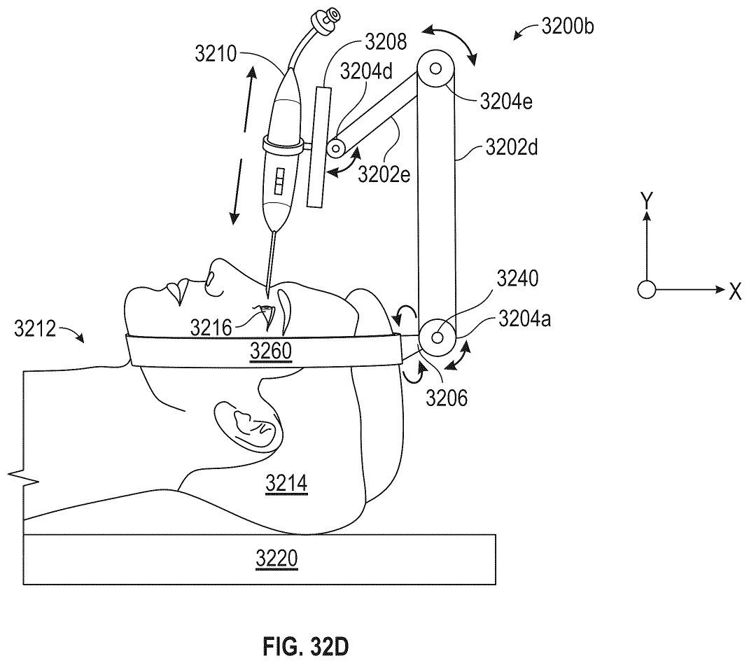

FIGS. 32A-32D illustrate side schematic views of exemplary support arms 3200a and 3200b for supporting a delivery device during a subretinal injection procedure, according to certain embodiments described herein. The support arms 3200a and 3200b may be utilized with any of the delivery devices and/or delivery systems as described herein without limitation.

In general, the support arms 3200a and 3200b are configured to support, or hold, a delivery device during a subretinal injection procedure such that the delivery device does not need to be held throughout the entire procedure by the surgeon or other surgical staff. This facilitates improved positioning of an injection needle within the patient's eye and reduces undesirable movements that would otherwise occur if the delivery device were being held by a user.

Looking to FIG. 32A, a first support arm 3200a is illustrated supporting a delivery device 3210 inserted into the eye 3216 of a patient 3212. The first support arm 3200a comprises a serial arm having a plurality of articulable links 3202 coupled by revolute joints 3204, which are lockable in rotational position. The links 3202 may generally comprise any suitable elongated and rigid members, and the revolute joints 3204 may generally comprise any suitable type of lockable revolute joints, including lockable pin or knuckle joints.

In the example shown, the support arm 3200a includes three links 3202a-3202c, wherein link 3202a comprises a most proximal link, the link 3202b comprises an intermediate link, and the link 3202c comprises a most distal link. However, the utilization of more or less links 3202 is further contemplated. For example, the utilization of more links 3202 (and thus, more revolute joints 3204) may facilitate more articulation points for the support arm 3200a.

The links 3202a-3202c are sequentially coupled to each other by two revolute joints 3204b and 3204c: revolute joint 3204b movably couples the most proximal link 3202a with the intermediate link 3202b, and revolute joint 3204c couples the intermediate link 3202b with the most distal link 3202c. The most proximal link 3202a is further movably coupled to a base 3206 via a revolute joint 3204a, while the most distal link 3202c is movably coupled to a delivery device adapter 3208 via a revolute joint 3204d. Each of the revolute joints 3204a-3204d is oriented to facilitate rotation of the links 3202 and/or delivery device adapter 3208 about horizontal axes parallel to a horizontal axis X in FIG. 32A. Meanwhile, the base 3206 may comprise a lockable rotary bearing, thereby facilitating rotation of the base 3206 about a vertical axis parallel to a vertical axis Y in FIG. 32A. Accordingly, the links 3202, revolute joints 3204, and base 3206 altogether facilitate at least three degrees-of-freedom for the delivery device 3210 when coupled to the delivery device adapter 3208.

FIG. 32B illustrates a magnified side schematic view of the delivery device adapter 3208 coupled to the revolute joint 3204d. As shown, the delivery device adapter 3208 includes a holder 3230 movably coupled to a rail 3232. The holder 3230 comprises any suitable type of grasping device configured to securely and removably grasp the delivery device 3210 (shown in phantom in FIG. 32B). In certain examples, the holder 3230 comprises a tubular or ring-like body through which the delivery device 3210 may be inserted and secured via friction or other locking mechanism. In other examples, the holder 3230 comprises a clamp. In still other examples, the holder 3230 comprises a clip.

The holder 3230 is configured to linearly translate (e.g., slide) along the rail 3232 in two opposing directions (represented by arrows 3218a and 3218b), which facilitates a longitudinal movement of the delivery device 3210 parallel to a major longitudinal axis A of the delivery device when the delivery device 3210 is coupled thereto. This one-dimensional translational movement of the holder 3230 may be controlled separately from the rest of the support arm 3200a, thus enabling fine-tuning of the longitudinal position of the delivery device 3210 when inserted into the eye 3216 of the patient 3212. For example, such translation movement of the holder 3230 may be utilized to precisely and cautiously position an injection needle of the delivery device 3210 between the RPE and the sensory retina. Translation of the holder 3230 may be controlled by any suitable mechanism, such as a turning knob or similar device. In certain embodiments, the holder 3230 may be locked in place after adjustment to a desired position along the rail 3232 by any suitable releasable locking means.

Turning back now to FIG. 32A, during use, the base 3206 of the support arm 3200a may be rotatably coupled, around a vertical rotational axis parallel to axis Y, to an operating table 3220 (or other support structure) upon which the patient 3212 is laid to perform a subretinal injection procedure. The fixation of the support arm 3200a to the operating table 3220 facilitates improved stability of the support arm 3200a during performance of the subretinal injection procedure, thereby minimizing any unintended movements thereof which would transfer to (i.e., cause movement of) the delivery device 3210. Accordingly, the risk of damage to the patient's eye 3216 as caused by unintended movements of the delivery device 3210 is curtailed, and the requisite skill level for performing the procedure relaxed.

In certain embodiments, the support arm 3200a is further configured to movably rest on the head 3214 of the patient 3212 during performance of the subretinal injection procedure. This may provide at least a partial intrinsic compensation of head movement by the patient 3212 during the procedure, since the support arm 3200a will move with the patient's head 3214 and transfer such motion to the delivery device 3210 removably coupled therewith. For example, any unconscious movements of the patient's head 3214 caused by the patient's respiration may be inherently transferred to the support arm 3200a, which then transfers the movement to the delivery device 3210. In FIG. 32A, the most proximal link 3202a is shown resting on the head 3214 of the patient 3212. In such an example, the revolute joint 3204a may not be locked in position during the subretinal injection procedure, thereby enabling free movement of the link 3202a relative to the base 3206, which may be locked in position about its own rotational axis. In still further embodiments, the support arm 3200a may be utilized while not resting on the head 3214 of the patient 3212. In such embodiments, the revolute joint 3204a may be locked in a position such that the link 3202a does not contact the patient's head 3214.

In certain embodiments, to manipulate the support arm 3200a into a desired position/orientation, each of the links 3202 may be manually adjusted by the surgeon prior to performing the subretinal injection with the delivery device 3210. In certain embodiments, the support arm 3200a is initially in a “locked” state, wherein rotation of the revolute joints 3204 is prevented and the links 3202 are therefore fixed in position. In such embodiments, in order to adjust the links 3202, the revolute joints 3204 must be first unlocked, thereby allowing free movement of each of the links 3202. In certain examples, each of the revolute joints 3204 may be individually released to enable movement of adjacent links 3202 only. In certain other examples, however, all of the revolute joints 3204 are simultaneously released, via a single mechanism or action, to enable movement of all the links 3202. The revolute joints 3204 may be released via any suitable mechanical or electronic mechanisms. For example, in certain embodiments, the revolute joints 3204 may be mechanically released via a push-push mechanism, push button, locking screw, or other mechanical means disposed on the support arm 3200a. In certain other embodiments, the revolute joints 3204 may be released via an electromechanical locking mechanism upon user input from a button or switch on the support arm 3200a, a foot pedal, or other user input device. In certain other embodiments, the revolute joints 3204 may be locked in position via the same or similar mechanisms as described above with reference to release of the revolute joints 3204.

Generally, the manipulation of the links 3202 may be accomplished prior to or after inserting the delivery device 3210 into the delivery device adapter 3208, and/or prior to or after inserting the delivery device 3210 into the eye 3216 of the patient 3212. For example, in certain embodiments, the delivery device 3210 is first inserted into the delivery device adapter 3208 of the support arm 3200a, after which the revolute joints 3204 are released and the links 3202 manipulated to steer the attached delivery device 3210 toward and into the eye 3216 of the patient 3212. At this point, the revolute joints 3204 are locked in place, and the holder 3230 of the delivery device adapter 3208 is finely adjusted to facilitate insertion of an injection needle of the delivery device 3210 into the subretinal space. The holder 3230 may then be locked in place, and injection fluids thereafter delivered to the subretinal space via the delivery device 3210.

In certain other embodiments, the delivery device 3210 is first inserted into the eye 3216 of the patient. Thereafter, the revolute joints 3204 of the support arm 3200a are released and the links 3202 manipulated to steer the delivery device adapter 3208 to the delivery device 3210 already inserted into the eye 3216. At this point, the delivery device adapter 3208 is attached to the delivery device 3210. The links 3202 may then be optionally finally adjusted prior to the revolute joints 3204 being locked. Afterwards, the holder 3230 of the delivery device adapter 3208 is finely adjusted and locked in place, and injection fluids are delivered to the subretinal space via the delivery device 3210.

In certain embodiments, the links 3202 are configured to be manually and freely adjusted by the surgeon. In certain embodiments, the links 3202 are configured to be adjusted via one or more knobs 3240 on each link 3202 or revolute joint 3204. In such embodiments, the knobs 3240 may comprise any suitable mechanical control mechanism for manipulating the corresponding link 3202, such as push buttons, switches, rotating knobs, or the like. In certain embodiments, the knobs 3240 are operably connected to, e.g., one or more gearwheels, for controlling the angle and/or movement of each link 3202 in one or more directions. In further embodiments, rather than physical knobs 3240, the links 3202 may be controlled by digital knobs as driven by an electronic controller, such as a controller in communication with a surgical console or other device comprising a computer. For example, the digital knobs may comprise digital controls as provided by a software interface of a computer, which when adjusted by the surgeon, send a signal to the electronic controller for driving manipulation of the links 3202 (e.g., via rotation of the revolute joints 3204). In such embodiments, the support arm 3200a may be a fully, or partially, robotic arm. In such embodiments, rather than controlling the support arm 3200a through a software interface, the support arm 3200 may be controlled via a joystick.

As noted above, FIGS. 32C and 32D illustrate side schematic views of another exemplary support arm 3200b. The support arm 3200b is substantially similar to the support arm 3200a in structure and function, but for a few aspects which are discussed below.

As shown in FIGS. 32C and 32D, support arm 3200b is movably coupled to the head 3214 of the patient 3212 via a band 3260 configured to be worn by the patient 3212. The fixation of the support arm 3200b to the head 3214 of the patient 3212 provides complete compensation for any head movement by the patient 3212 during the procedure, since the support arm 3200b will move/rotate with the patient's head 3214 and transfer such motion to the delivery device 3210 removably coupled therewith. Thus, any unconscious movements of the patient's head 3214 caused by the patient's respiration may be directly transferred to the support arm 3200b, which then transfers the movement to the delivery device 3210. In FIG. 32C, the band 3260 comprises a headband that is configured to be secured around the forehead and crown of the head 3214 of the patient 3212. In FIG. 32D, the band 3260 is configured to be secured around the chin and the top of the head 3214 of the patient 3212.

In each of FIGS. 32C and 32D, the support arm 3200ba is coupled to the band 3260 via the base 3206, which is rotatably attached to the band 3260. Further, the support arm 3200b only comprises two links 3202: a most proximal link 3202d and a most distal link 3202e, which are movably coupled together via a revolute joint 3204e. In these two examples, the support arm 3200b may not necessitate as many links 3202 as a standalone support arm configured to be supported off the head 3214 of the patient 3212, such as support arm 3200a. However, even with fewer links 3202, the support arm 3200b may function substantially the same as support arm 3200a. Even still, the utilization of more or less links 3202 for the support arm 3200b is further contemplated.

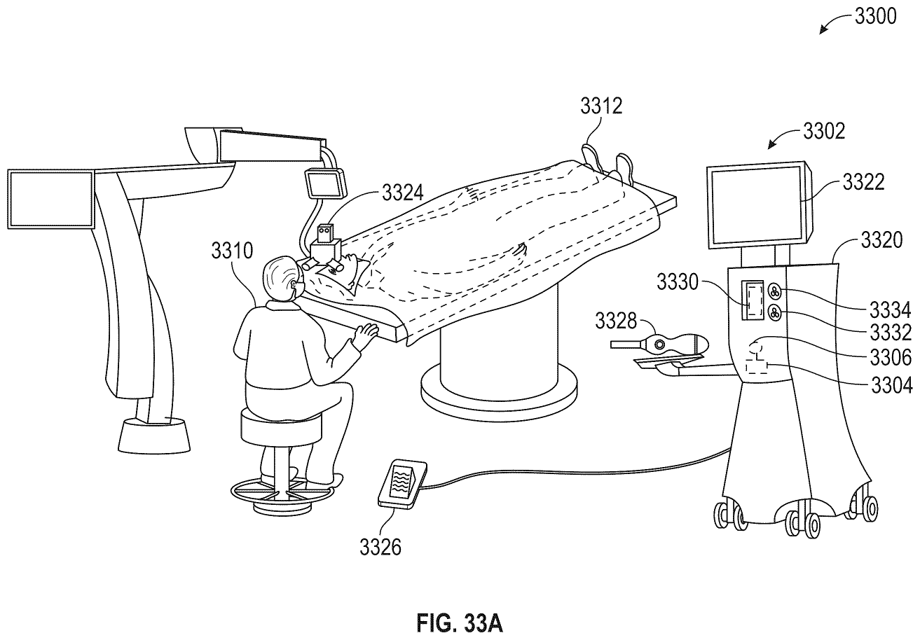

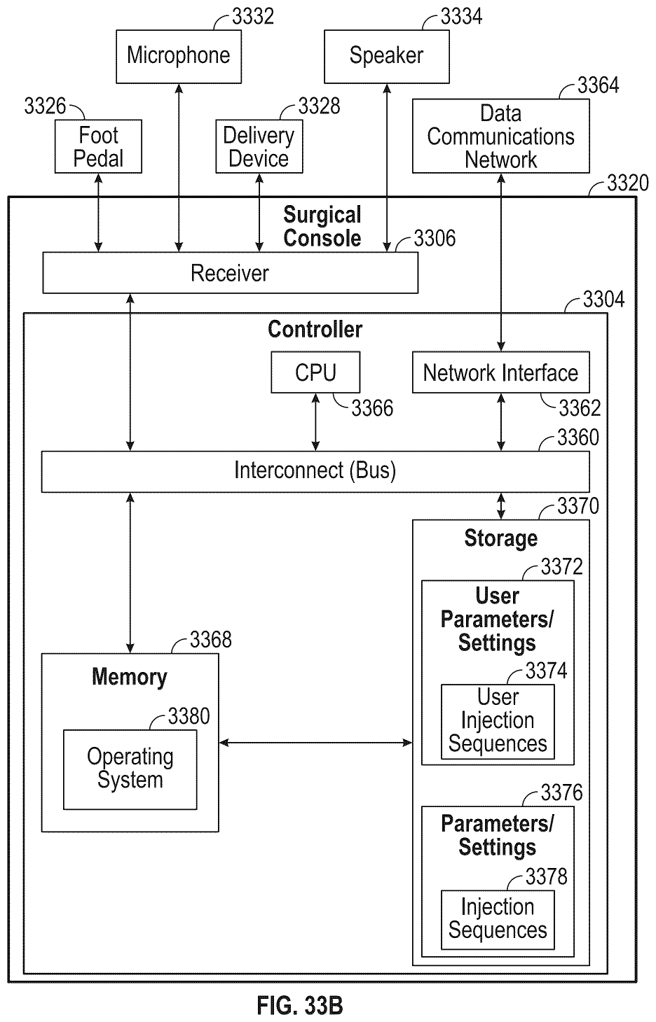

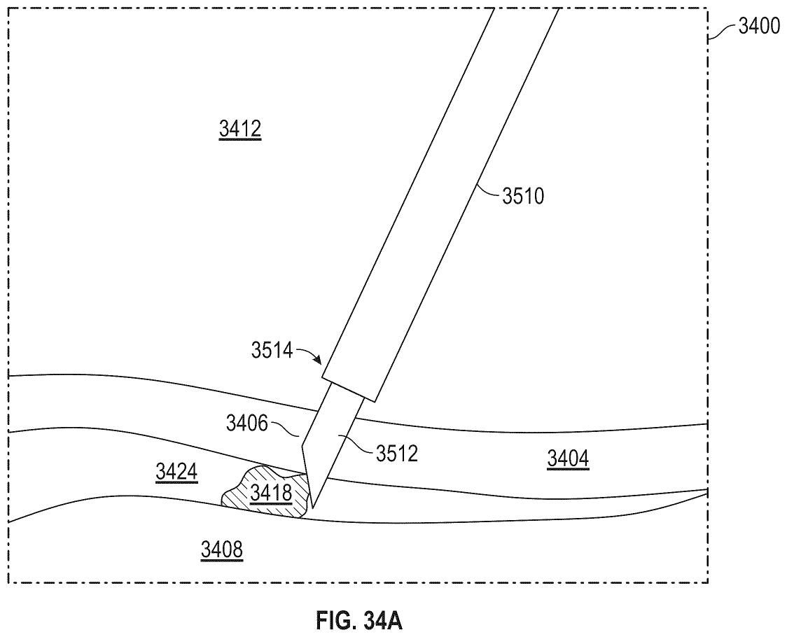

FIG. 33A illustrates an example operating environment 3300, such as an ophthalmic operating environment, during the performance of a subretinal injection procedure, according to certain embodiments of the present disclosure. As described above, subretinal injection procedures are typically very delicate procedures since they require the puncturing and/or manipulation of one or more tissues/membranes of the eye to access the subretinal space. Accordingly, such procedures require a great amount of skill by the surgeon to minimize the risk of unnecessary injury to a patient's eye. In addition to precisely positioning the delivery device and/or other surgical tools within the patient's eye, the surgeon must carefully control the flow and volume of fluids delivered to the subretinal space. Delivering too much fluid and/or delivering fluids too quickly may cause unwanted trauma to the tissues on either side of the subretinal space (e.g., the retina and RPE). Meanwhile, delivering too little fluid (e.g., too little therapeutic substance) may reduce the efficacy of the procedure. Thus, control over the volume and flow of delivered fluids is critical to a successful subretinal injection procedure. The below description provides systems and methods of improve volume and flow control of fluids delivered during a subretinal injection. Such systems and methods may be utilized, without limitation, in combination with the delivery systems and delivery devices described elsewhere herein.

As shown in FIG. 33A, the operating environment 3300 includes a surgeon 3310, patient 3312, and a surgical system 3302, which may be representative of the surgical system 400 described above with reference to FIG. 4. Accordingly, the surgical system 3302 includes a variety of systems and tools, such as a surgical console 3320, a display device 3322, a microscope system 3324, a foot pedal 3326, and a delivery device 3328, which may comprise any of the delivery devices and/or delivery systems described herein. In certain embodiments, the surgical system 3302 further includes a fluidic drive system 3330, which is configured to drive the flow of injection fluids during a subretinal injection fluids and may be disposed, for example, within the surgical console 3320. An example of a console configured for performing subretinal injection procedures is the Constellation® System available from Alcon Laboratories, Inc., Fort Worth, Texas.

Surgical console 3320 also includes controller 3304 (shown in phantom), and in certain embodiments, a receiver 3306 in communication with the controller 3304. The controller 3304 is configured to cause (e.g., control) surgical console 3320 to perform one or more tasks for driving a subretinal injection procedure, such as of driving the flow of injection fluids via the fluidic drive system 3330, according to inputs from the surgeon 3310, and/or stored settings and parameters associated with the procedure type, the surgeon 3310, and/or the patient 3312. In certain embodiments, the controller 3304 interfaces with a digital interface of the fluidic drive system 3330, which can be controlled by digital commands from the controller 3304.

The receiver 3306 may include any suitable interface for communication (e.g., one-way or two-way signals) between controller 3304 and, e.g., foot pedal 3326 and/or delivery device 3328. For example, receiver 3306 may include a wireless or wired connection between controller 3304 and foot pedal 3326 and/or delivery device 3328. In certain embodiments, the receiver 3306 is also in communication with a microphone 3332, which is configured to receive speech commands from the surgeon 3310 and/or other surgical staff and convert them into signals that are processed and utilized by the controller 3304 for performing the one or more tasks for driving the subretinal injection procedure. Although depicted on the surgical console 3320, the microphone 3332 may be disposed in any suitable position within the operating environment 3300.

In the embodiments of FIG. 33A, the controller 3304 and receiver 3306 are integrated within surgical console 3320, wherein controller 3304 includes or refers to one or more processors and/or memory devices integrated within the surgical console. In certain other embodiments, controller 3304 and/or receiver 3306 are stand-alone devices or modules that are in wireless or wired communication with, e.g., surgical console 3320 and other devices within operating environment 3300. In certain embodiments, the controller 3304 refers to a set of software instructions that a processor associated with surgical console 3320 is configured to execute. In certain aspects, operations of controller 3304 may be executed partly by the processor associated with controller 3304 and/or surgical console 3320 and partly in a public or private cloud.

The controller 3304 interfaces (e.g., wirelessly or wired) with, e.g., the foot pedal 3326, the delivery device 3328, and/or the fluidic drive system 3330 during a subretinal injection procedure to control various parameters associated with fluidic flow of injection fluids. Such parameters, hereinafter referred to as “fluid flow parameters,” include fluid flow rate, fluid pressure, fluid delivery volume, fluid delivery time, as well as other parameters associated with the flow of injection fluids into or out of the eye of the patient 3312 during the subretinal injection procedure. The fluid flow parameters may be measured directly by the fluidic drive system 3330, or by a fluid flow sensor or other type of sensor separate from the fluidic drive system, and thereafter provided to the controller 3304. For example, in embodiments where the fluidic drive system 3330 comprises a motor-controlled syringe, the fluidic drive system 3330 may indicate to the controller 3304 a distance that a plunger of the syringe has been translated in relation to time, or a position of the plunger in relation of time, and such information can be processed by the controller 3304 to determine the various fluid flow parameters.

In certain embodiments, the controller 3304 controls the fluid flow parameters according to stored settings associated with the procedure type, the surgeon 3310, and/or the patient 3312. For example, in certain embodiments, prior to a subretinal injection procedure, the surgeon 3310 may program the controller 3304 with one or more injection sequences for the particular subretinal injection procedure to be performed, and/or the particular patient 3312. Such injection sequences may then be initiated during the subretinal injection procedure, and may include temporal sequences of desired fluid flow parameters. Generally, the injection sequences may include static settings of fluid flow parameters, such as a constant flow rate and/or a constant fluid pressure, or dynamic settings of fluid flow parameters, such as a varying flow rate and/or a varying fluid pressure, in relation to time. Utilization of programmed injection sequences with predetermined temporal sequences of desired fluid flow parameters facilitates accurate and precise (i.e., repeatable) performance of subretinal injection procedures by the surgeon 3310. And, each subretinal injection procedure may be performed according to specific needs of the surgeon 3310 and/or the patient 3312, thereby improving the efficiency and efficacy of each procedure. Further, such injection sequences improved volume and pressure control when injecting fluids into the subretinal space, thereby reducing the risk of damage to the retina and RPE.

In certain embodiments, the programmed injection sequences define various settings related to fluid flow parameters. Such settings include: a maximum and/or minimum injection fluid flow rate; a maximum and/or minimum injection fluid volume; a maximum and/or minimum injection time; a maximum and/or minimum injection fluid pressure; a maximum and/or minimum rate of change between fluid flow rates, injection fluid volumes, injection fluid pressures; a number, time, and/or sequence of injection phases, and the like.

In certain embodiments, one or more programmed injection sequences may be simultaneously or sequentially selected, activated, and/or inactivated by the surgeon 3310 (and/or other surgical staff) via inputs received from the surgeon 3310 (and/or other surgical staff) by the foot pedal 3326, delivery device 3328, and/or microphone 3332. For example, in such embodiments, the inputs may include the manipulation, by the surgeon 3310, of hand-actuated controls, such as a button or other toggle, on the delivery device 3328, and/or the manipulation of foot-actuated controls, such as a treadle, on the foot pedal 3326. In certain examples, the inputs may include speech commands received by the microphone 3332 from the surgeon 3310. The utilization of speech commands for fluid flow control may facilitate easier handling of the delivery device 3328 and/or other surgical tools during a subretinal procedure, as no additional motor coordination is needed by the surgeon 3310 to adjust or control fluid flow parameters. Accordingly, the surgeon 3310 may focus their full attention to maintaining the delivery device 3328 motionless during injection.

Signals corresponding to the inputs on the delivery device 3328, foot pedal 3326, and/or microphone 3332 are received by the receiver 3306 and communicated to the controller 3304, which then takes one or more actions for controlling the driving of fluids by the fluidic drive system 3330 according the inputs from the surgeon 3310 (and/or other surgical staff) and the programmed injection sequences. In certain embodiments, the inputs from the surgeon 3310 are mapped, by the controller 3304, to corresponding injection sequences, fluid flow parameters, and/or actions to be perform by the fluidic drive system 3330 and/or surgical console 3320, after which the controller 3304 configures and drives the fluidic drive system 3330 and/or surgical console 3320 to perform such injection sequences, fluid flow parameters, and/or actions. In certain embodiments, the injection sequences, fluid flow parameters, and/or actions being taken or to be taken are displayed on display device 3322 for the surgeon 3310.

In certain embodiments, the programmed injection sequences comprise user-programmed (e.g., surgeon-programmed) injection sequences that are programmed prior to the performance of a subretinal injection procedure. In certain embodiments, in addition to or as an alternative to the injection sequences programmed by the surgeon 3310, the controller may comprise one or more pre-programmed and universal injection sequences and/or other settings associated with subretinal injection procedures. Such pre-programmed and universal sequences may include injection sequences generally applicable to a majority of subretinal injection procedures, and may be provided (e.g., programmed) by a manufacturer of one or more components of the surgical system 3302 during fabrication or assembly. In such embodiments, the inputs from the surgeon 3310 during a procedure are mapped, by the controller 3304, to the corresponding pre-programmed and universal injection sequences and/or other settings to be perform by the fluidic drive system 3330 and/or surgical console 3320, after which the controller 3304 configures and drives the fluidic drive system 3330 and/or surgical console 3320 to perform according to such pre-programmed and universal injection sequences and/or other settings.

In certain embodiments utilizing programmed or pre-programmed injection sequences, the controller 3304 comprises a safeguard to halt the flow of injection fluids as driven by the fluidic drive system 3330 during an activated injection sequence. For example, in certain embodiments, the controller 3304 may be programmed to require a continuous manual input from the surgeon 3310 in order for an activated injection sequence to be continued or performed. In other words, the controller 3304 may comprise a “dead man's switch,” which stops performance of the injection procedure in the absence of input from the surgeon 3310. The continuous manual input may comprise the continuous manipulation of one or more foot-actuated controls on the foot pedal 3326, and/or the continuous manipulation of one or more hand-actuated controls on the delivery device 3328. For example, in order for an injection sequence to be initiated and carried out to completion, the surgeon 3310 may be required to depress a treadle on the foot pedal 3326 during the entire injection sequence. Accordingly, if any issues arise during the injection sequence, the surgeon 3310 may release the treadle, causing the injection sequence to stop immediately. In such examples, the utilization of the foot pedal 3326 as the dead man's switch, rather than another device in the operating environment 3300, may reduce any unnecessary visual distractions for the surgeon 3310 during the injection procedure, and the surgeon 3310 may better focus their visual attention to the eye of the patient 3312 and their maneuvering of the delivery device 3328 therein.

In further embodiments, the controller 3304 may control the fluid flow parameters entirely or partially based on real-time inputs from the surgeon 3310 (and/or other surgical staff), thus enabling entire manual control of the subretinal injection procedure. Similarly, as above, the inputs may include the manipulation, by the surgeon 3310, of hand-actuated controls on the delivery device 3328, and/or the manipulation of foot-actuated controls on the foot pedal 3326. In such examples, a degree or level of manipulation of the hand- or foot-actuated controls (e.g., a degree or amount of depression) may correspond with a magnitude of a fluid flow parameter. For example, further depressing a hand- or foot-actuated control may cause an increase in fluid flow rate, fluid pressure, and/or fluid delivery volume, while reducing the depression of (or releasing) the hand- or foot-actuated control may cause a decrease in fluid flow rate, fluid pressure, and/or fluid delivery volume. In certain examples, the inputs may include speech commands received by the microphone 3332 from the surgeon 3310.

Signals corresponding to the inputs on the delivery device 3328, foot pedal 3326, and/or microphone 3332 are received by the receiver 3306 and communicated to the controller 3304, which then takes one or more actions for driving the fluidic drive system 3330 according the inputs from the surgeon 3310 (and/or other surgical staff).

In certain embodiments, during the performance of the subretinal injection procedure, the surgical system 3302 may provide visual and/or auditory feedback to the surgeon 3310 and/or other surgical staff relating to the fluid flow parameters and the progress of the procedure. For example, in certain embodiments, measurements of the fluid flow parameters (as a volume unit (e.g., p L (microliter)) or percentage of volume (e.g., %)), and/or a status or progress of an injection sequence, may be continuously or periodically displayed on a screen of the display device 3322, and/or on a screen or ocular of the microscope system 3324. In certain embodiments, the surgical system 3302 may comprise a speaker 3334 for providing a periodic audible indicator to the surgeon 3310 regarding the fluid flow parameters and/or the progress of the procedure. In such embodiments, the audible indicators may facilitate easier handling of the delivery device 3328 and/or other surgical tools during a subretinal procedure, as the number of visual distractions during the procedure are reduced or limited. Accordingly, the surgeon 3310 may focus their full visual attention to maintaining the delivery device 3328 motionless during injection. Examples of suitable audible indicators include speech as well as non-speech sounds. Where non-speech sounds are utilized, a type, frequency, amount, or tone of the non-speech sounds may indicate different parameters and/or statuses of the subretinal injection procedure. In certain embodiments, audible indicators may be periodically provided during predefined intervals based on time or volume of fluids injected. Such audible indicators may indicate measurements of the fluid flow parameters as a volume unit (e.g., μL) or percentage of volume (e.g., %).

FIG. 33B illustrates an exemplary diagram showing how various components of operating environment 3300, shown in FIG. 33A, communicate and operate together. As shown, the surgical console 3320 of surgical system 3302 includes, without limitation, the controller 3304 and receiver 3306, which enables connection of the controller 3304 to the foot pedal 3326, the delivery device 3328, and/or the fluidic drive system 3330. The controller 3304 includes an interconnect 3360 and a network interface 3362 for connection with a data communications network 3364. The controller 3304 further includes a central processing unit (CPU) 3366, memory 3368, and storage 3370. The CPU 3366 may retrieve and store application data in the memory 3368, as well as retrieve and execute instructions stored in the memory 3368. The interconnect 3360 transmits instructions and application data, such as instruction related to the control of fluid flow parameters, among the CPU 3366, network interface 3362, memory 3368, storage 3370, delivery device 3328, fluidic drive system 3330, etc. The CPU 3366 can represent a single CPU, multiple CPUs, a single CPU having multiple processing cores, and the like. The memory 3368 represents random access memory.

The storage 3370 may be a disk drive. Although shown as a single unit, the storage 3370 may be a combination of fixed or removable storage devices, such as fixed disc drives, removable memory cards or optical storage, network attached storage (NAS), or a storage area-network (SAN). The storage 3370 may comprise user-programmed subretinal injection procedure parameters/settings 3372, such as user-programmed injection sequences 3374. The storage 3370 may further comprise pre-programmed subretinal injection procedure parameters/settings 3376, such as pre-programmed universal injection sequences 3378. Each of the user-programmed injection sequences 3374 and the pre-programmed universal injection sequences 3378 may comprise pre-set instructions for controlling fluid flow parameters as driven by the fluidic drive system 3330.

Meanwhile, the memory 3368 includes an operating system 3380 and/or one or more applications, which when executed by the CPU 3366, allow the controller 3304 to configure and operate the surgical console 3320 (e.g., including fluidic drive system 3330 based on retrieved subretinal injection procedure parameters/settings).

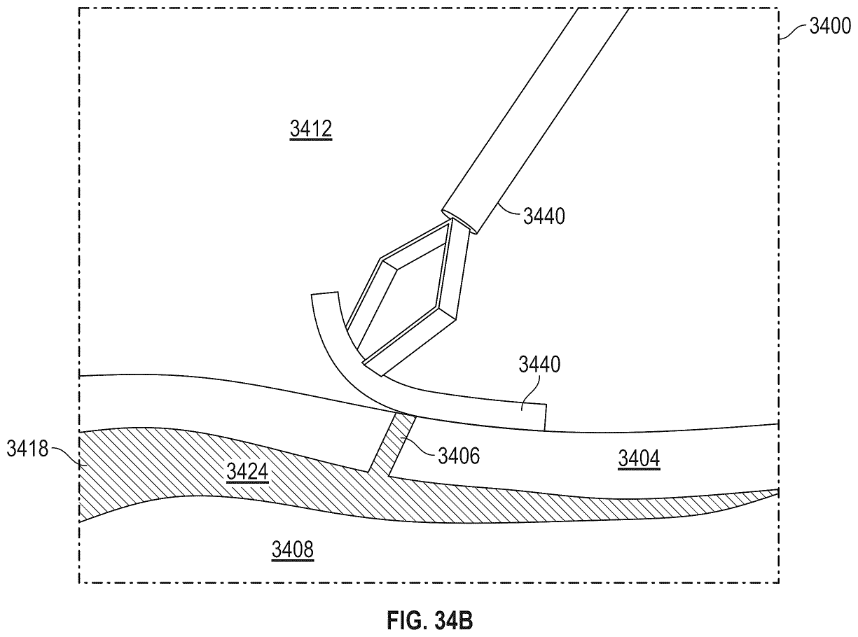

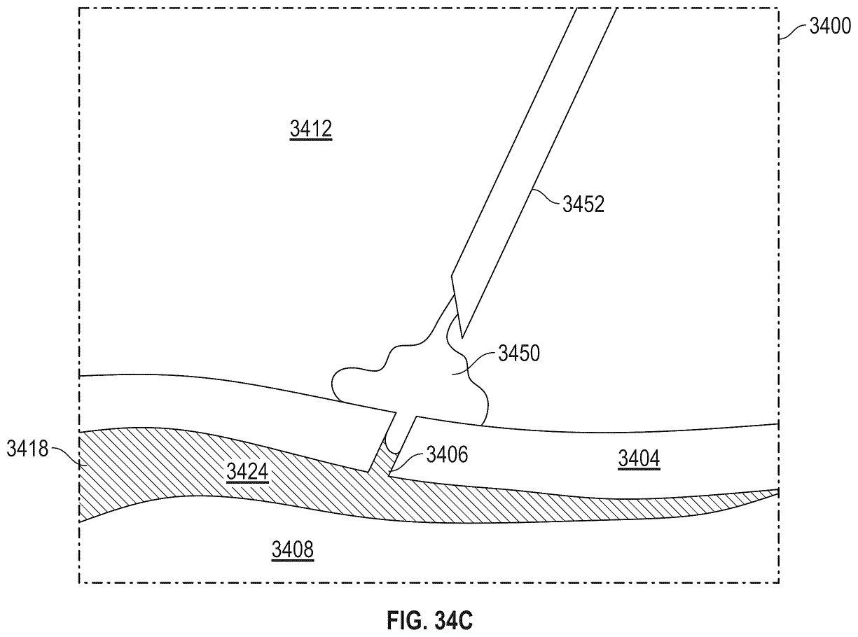

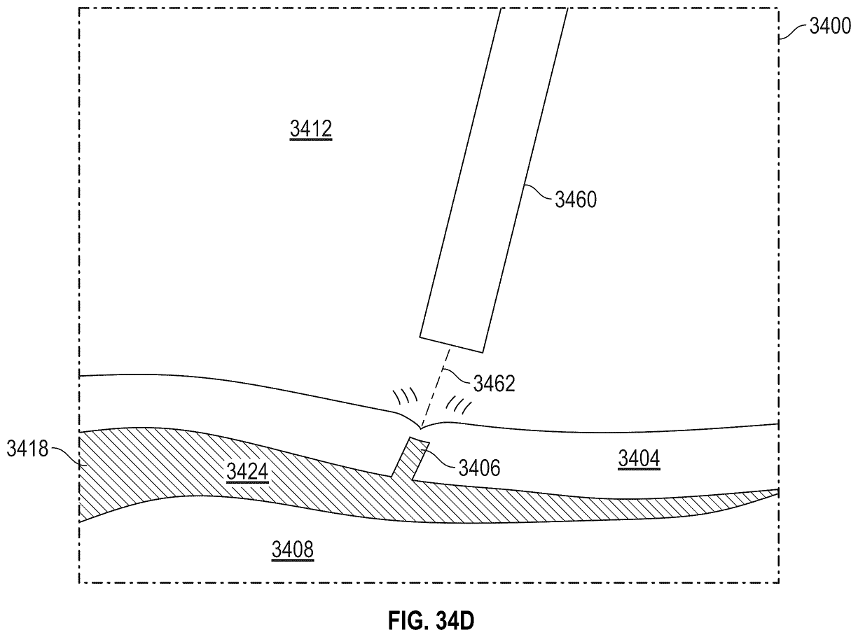

FIGS. 34A-34D illustrate transverse sectional views of a portion of an eye 3400 at different steps of performing an exemplary subretinal injection procedure with post-injection sealing, according to certain embodiments of the present disclosure. During and after injection of fluids to the subretinal space at a target injection site, there may be some leakage, or spilling, of the non-treatment and/or treatment solutions through the target injection site. This is typically undesirable, since the escape of any therapeutic substances reduces the efficacy of the procedure and may also increase the risk of undesired complications resulting from the therapeutic substance contacting non-target ocular tissues. Thus, as discussed elsewhere herein, upon delivering fluids to the subretinal space at the target injection site, the target injection site may be filled with a sealing agent to prevent the injected fluids from escaping the subretinal space. The below description comprises one example of performing such a sealing procedure after subretinal delivery of fluids using a transvitreal approach. Although a transvitreal approach is described, aspects of the below method may be applied utilizing a suprachoroidal approach.

Turning now to FIG. 34A, a transvitreal subretinal injection is performed utilizing any suitable delivery devices and/or systems described herein. For example, an injection cannula 3510 of a delivery device may be inserted through a valved insertion cannula (or other entry cannula) disposed through a sclerotomy of the eye 3400 and guided through the vitreous chamber 3412 toward the retina 3404. The injection cannula 3510 of the delivery device is guided through the vitreous chamber 3412 until a distal end 3514 thereof is positioned adjacent to a target injection site 3406 on a surface of the retina 3404. Once in position, an injection needle 3512 of the delivery device may be extended and/or inserted through target injection site 3406 and into the subretinal space 3424, e.g., between the outermost neural layer of the retina 3404 and the retinal pigment epithelium (RPE) 3408 to inject fluids 3418 into the subretinal space 3424. Thereafter, the injection needle 3512 may be retracted into the cannula 3510, and the cannula 3510 removed from the eye 3400 through the valved insertion cannula.

At this point, sealing of the target injection site 3406 using any one of a plurality of sealing modalities may be performed. FIGS. 34B-34D illustrate various sealing modalities that may be utilized in combination with a subretinal injection procedure.

As shown in FIG. 34B, in certain embodiments, a graft 3440 is applied over the target injection site 3406 using a suitable applicator device, such as forceps 3442. For example, the forceps 3442 may be utilized to grasp and insert the graft 3440 into the eye 3400 (e.g., through the valved cannula or another entry cannula in the sclera), and then position and flatten the graft 3440 over the target injection site 3406 such that it seals the target injection site 3406.

In certain embodiments, the graft 3440 comprises a biological graft or scaffold, such as a cellular graft. In certain embodiments, the graft 3440 comprises a human amniotic membrane (hAM) graft. Amniotic membrane, or amnion, is the innermost layer of the placenta and consists of a non-sticky basement membrane, a thick intermediate collagen layer, and a sticky avascular stromal matrix. For sealing purposes, the sticky stromal matrix of the amniotic membrane may be placed “face-down” on the surface of the retina 3404 to adhere to the retina 3404 and seal the target injection site 3406. Other examples of biological scaffolds or cellular grafts that may be utilized include scaffolds or grafts comprising retinal cells, such as iPSC-derived retinal cells.

In certain embodiments, the graft 3440 comprises a polymer-based scaffold, such as a polymeric nanofiber scaffold.

Turning now to FIG. 34C, as an alternative to a graft, a sealing solution 3450 may be applied over the target injection site 3406 using a suitable applicator device, such as injector 3452. In certain embodiments, the sealing solution 3450 may comprise one or more human proteins and/or cellular attachment factors in solution that can be injected at the target injection site 3406 to seal the target injection site 3406. Examples of proteins and attachment factors that may be utilized include fibrin, collagen, thrombin, fibronectin, laminin, as well as other proteins and/or attachment factors facilitating coagulation and/or adhesion. After injection, the proteins and/or attachment factors may be naturally broken down in vivo by the patient's own catabolism pathways/processes.

In certain embodiments, the sealing solution 3450 comprises a polymer that can be naturally degraded in vivo by the patient's own catabolism pathways/processes. For example, in certain embodiments, the sealing solution 3450 may comprise a polymeric hydrogel, such as a biopolymer. Examples of biopolymers that may be utilized include chitosan, hyaluronic acid, gelatin, alginate, methylcellulose, and collagen.

FIG. 34D illustrates yet another alternative sealing modality. In FIG. 34D, the target injection site 3406 in the retina 3404 is sealed via photocoagulation by a laser probe 3460. The laser probe 3460 may thus include any suitable type of retinal treatment laser probe operably coupled to a laser source for generating and propagating a laser beam with a wavelength between about 400 and about 850 nm. For example, the laser probe 3460 may be operably coupled to an Nd-YAG laser source. Accordingly, the laser probe 3460 may be used to transmit a laser beam 3462 at the target injection site 3406, which may cauterize and seal the retina 3404 at the target injection site 3406, thereby preventing any previously delivered therapeutic substances from escaping.

In summary, embodiments of the present disclosure improve the efficacy, efficiency, and safety of subretinal injection for treatment of ophthalmic conditions.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

### Example Embodiments

Embodiment 1: A surgical instrument for fluid injection, the surgical instrument comprising: a handpiece configured for grasping by a user, the handpiece comprising: a first lumen disposed therein; and an actuatable toggle movably coupled with the handle; a cannula coupled to the handpiece and configured to be introduced into an eye, the cannula comprising a second lumen extending therethrough; and a needle movably disposed within the second lumen and coupled with the actuatable toggle, the needle configured to extend from and retract into the second lumen at a distal end of the cannula upon actuation of the toggle.

Embodiment 2: The surgical instrument of Embodiment 1, wherein the needle comprised a curved needle, and wherein a curvature of the needle increases as it is extended from the second lumen, and wherein a curvature of the needle decreases as it is retracted into the second lumen.

Embodiment 3: The surgical instrument of Embodiment 2, wherein the needle is formed of an elastic material.

Embodiment 4: The surgical instrument of Embodiment 1, further comprising an annular insert disposed in the second lumen at the distal end of the cannula, the annular insert circumscribing at least a portion of the needle within the second lumen, wherein extension of the needle from the second lumen and through the annular insert increases a flexibility of the needle, and wherein retraction of the needle into the second lumen and through the annular insert increases a stiffness of the needle.

Embodiment 5: The surgical instrument of Embodiment 1, wherein the needle comprises: a first proximal portion having a first outer diameter; and a second distal portion comprising a second outer diameter.

Embodiment 6: The surgical instrument of Embodiment 5, wherein the first proximal portion has a gauge of 38 or smaller, and wherein the second distal portion has a gauge of 37 or larger.

Embodiment 7: The surgical instrument of Embodiment 5, wherein the first proximal portion has a gauge of 41 or smaller, and wherein the second distal portion has a gauge of 40 or larger.

Embodiment 8: The surgical instrument of Embodiment 1, wherein the needle comprises a beveled distal tip, the beveled distal tip comprising a distal endface disposed at a non-normal and non-zero angle relative to a major longitudinal axis of the needle.

Embodiment 9: The surgical instrument of Embodiment 8, wherein the needle further comprises a port disposed in a side wall thereof and adjacent the beveled distal tip.

Embodiment 10: The surgical instrument of Embodiment 1, wherein the needle comprises an annular sealing element circumscribing a portion of the needle at a distal end thereof.

Embodiment 11: The surgical instrument of Embodiment 1, wherein the needle comprises a polymeric coating formed on an inner wall thereof for reducing fluidic resistance through the cannula.

Embodiment 12: The surgical instrument of Embodiment 11, wherein the polymeric coating is further disposed on an inner wall of the cannula.

Embodiment 13: The surgical instrument of Embodiment 11, wherein the polymeric coating comprises at least one of poly(ethylene oxide) (PEO), poly(methyl methacrylate) (PMMA), poly(hydroxyethyl methacrylate) (PHEMA), polytetrafluoroethylene (PTFE), ethylene tetrafluoroethylene (ETFE), perfluoroalkoxy (PFA), chlorotrifluoroethylene (E-CTFE), or polyether ether ketone (PEEK).

Embodiment 14: The surgical instrument of Embodiment 1, wherein the actuatable toggle is lockable such that the needle may be fixed in either an extended or retracted position.

Embodiment 15: The surgical instrument of Embodiment 1, further comprising: a first flexible tubing fluidically coupled at a distal end of the first flexible tubing to the actuatable toggle or needle in the first lumen, the first flexible tubing further fluidically coupled at a proximal end of the first flexible tubing to a connector disposed at a proximal end of the handpiece, wherein the connector is further configured to fluidically couple to a second flexible tubing external to the first lumen.

Embodiment 16: The surgical instrument of Embodiment 15, further comprising: the second flexible tubing fluidically coupled to the connector external to the first lumen.

Embodiment 17: The surgical instrument of Embodiment 1, wherein the actuatable toggle is disposed around a circumference of the handpiece.

Embodiment 18: The surgical instrument of Embodiment 1, wherein the actuatable toggle comprises a plurality of toggles circumscribing a portion of the handpiece.

Embodiment 19: The surgical instrument of Embodiment 1, wherein the handpiece is configured to removably receive a cartridge prefilled with injection fluid, wherein the cartridge fluidically couples with the cannula or the needle for injection of the injection fluid.

Embodiment 20: A surgical instrument for fluid injection, the surgical instrument comprising: a handpiece configured for grasping by a user, the handpiece comprising: a first lumen disposed therein; and an actuatable toggle movably coupled with the handle; a cannula coupled to the handpiece and configured to be introduced into an eye, the cannula comprising a second lumen extending therethrough; and a needle movably disposed within the second lumen and coupled with the actuatable toggle, the needle configured to extend from and retract into the second lumen at a distal end of the cannula upon actuation of the toggle.

Embodiment 21: The surgical instrument of Embodiment 20, wherein at least a portion of the cannula is formed from a flexible metallic or thermoplastic material.

Embodiment 22: The surgical instrument of Embodiment 21, wherein another portion of the cannula is formed from a rigid material.

Embodiment 23: The surgical instrument of Embodiment 20, wherein the cannula comprises an elliptical, pill-shaped, or crescent-shaped cross-sectional profile.

Embodiment 24: The surgical instrument of Embodiment 20, wherein the cannula comprises a pre-formed curvature along a length of the cannula.

Embodiment 25: The surgical instrument of Embodiment 20, wherein the cannula further comprises a distal tip, the distal tip comprising a distal portion having a semi-circular and disc-like morphology for delaminating tissues upon insertion into the eye.

Embodiment 26: The surgical instrument of Embodiment 25, wherein the distal tip further comprises a proximal portion having a port through which the needle may be extended from and retracted into the second lumen.

Embodiment 27: The surgical instrument of Embodiment 26, wherein the proximal portion of the distal tip further comprises a sloped surface disposed adjacent to the port and along which the needle is configured to slide when being extended and retracted through the port.

Embodiment 28: The surgical instrument of Embodiment 20, wherein the cannula further comprises a distal tip formed of a photoluminescent material.

Embodiment 29: The surgical instrument of Embodiment 28, wherein the cannula further comprises a distal tip formed of a phosphorescent material.

Embodiment 30: The surgical instrument of Embodiment 20, wherein the cannula further comprises a distal tip comprising a spatula portion and a body portion, wherein a thickness of the distal tip increases between the spatula portion and the body portion to form a ramp for delaminating tissues upon insertion into the eye.

Embodiment 31: The surgical instrument of Embodiment 30, wherein body portion comprises a port through which the needle may be extended from and retracted into the second lumen.

Embodiment 32: The surgical instrument of Embodiment 31, wherein the body portion of the distal tip further comprises a sloped surface disposed adjacent to the port and along which the needle is configured to slide when being extended and retracted through the port.

Embodiment 33: The surgical instrument of Embodiment 20, further comprising a light-propagating fiber extending along the cannula and having a terminal end at or near a distal tip of the cannula, the light-propagating fiber configured to emit light from the terminal end.

Embodiment 34: The surgical instrument of Embodiment 33, wherein the light-propagating fiber comprises a single- or multi-core optical fiber configured to propagate white light.

Embodiment 35: The surgical instrument of Embodiment 20, wherein the cannula further comprises a distal tip, the distal tip comprising a port through which the needle may be extended from and retracted into the second lumen, the port disposed adjacent to a sloped surface for guiding the needle through the port when the needle is extended from and retracted into the second lumen.

Embodiment 36: The surgical instrument of Embodiment 35, wherein a proximal end of the needle is coupled to a sliding block, the sliding block configured to slide along the sloped surface when the needle is extended and retracted through the port.

Embodiment 37: The surgical instrument of Embodiment 36, wherein at least one of the sliding block and the sloped surface are formed of a material comprising at least one of steel, titanium, PEEK (polyetheretherketone), polyoxymethylene (POM), and polytetrafluoroethylene (PTFE).

Embodiment 38: The surgical instrument of Embodiment 20, wherein the cannula further comprises a port through which the needle may be extended from and retracted into the second lumen, the port disposed in a sidewall of the cannula, and wherein a distal portion of the needle comprises a corkscrew shape to enable extension and retraction of the needle through the port upon rotation of the needle.

Embodiment 39: The surgical instrument of Embodiment 38, wherein the needle is curled along a plane perpendicular to a major longitudinal axis of the needle to form the corkscrew shape.

Embodiment 40: The surgical instrument of Embodiment 20, wherein the cannula further comprises a distal tip comprising: a first port disposed in a sidewall of the distal tip through which the needle may be extended from and retracted into the second lumen, the port disposed adjacent to a sloped surface for guiding the needle through the port when the needle is extended from and retracted into the second lumen; and a second port disposed in a distal surface for injecting fluids along a flow path parallel or substantially parallel to a major longitudinal axis of the distal tip.

Embodiment 41: The surgical instrument of Embodiment 40, wherein the second port is fluidically coupled to fluidic tubing disposed within the cannula.

Embodiment 42: The surgical instrument of Embodiment 20, wherein the cannula further comprises a third lumen extending through at least a portion of a length of the cannula, the third lumen configured to removable receive a wire through a port disposed in the distal end of the cannula or in a sidewall of the cannula.

Embodiment 43: The surgical instrument of Embodiment 42, wherein the wire is configured to increase a stiffness of the cannula for insertion into the eye.

Embodiment 44: The surgical instrument of Embodiment 42, wherein the wire is configured to facilitate guidance of the cannula to a target injection site upon insertion into the eye.

Embodiment 45: A support system for a fluid injection device, the support system comprising: a support arm, comprising: a base configured to rotate about an axis thereof; a plurality of articulable links movably coupled to the base; a device adapter movably coupled to at least one link of the plurality of articulable links, the device adapter configured to secure the fluid injection device, and a plurality of revolute joints movably coupling the at least one link of the plurality of articulable links to the device adapter, adjacent links of the plurality of articulable links and at least another one link of the plurality of articulable links to the base.

Embodiment 46: The surgical instrument of Embodiment 45, wherein the base is rotatably coupled to a band configured to be placed around the head of a patient.

Embodiment 47: The surgical instrument of Embodiment 45, wherein the base is rotatably coupled to an operating table or other support structure configured to support the head of a patient.

Embodiment 48: The surgical instrument of Embodiment 47, wherein the base is configured to rotate about a vertical axis, and wherein at least one of the plurality of revolute joints is configured to rotate about a horizontal axis perpendicular to the vertical axis.

Embodiment 49: The surgical instrument of Embodiment 47, wherein at least one link of the plurality of articulable links is configured to be disposed against the head of the patient during use.

Embodiment 50: The surgical instrument of Embodiment 45, wherein the plurality of articulable links comprise a series of articulable links.

Embodiment 51: The surgical instrument of Embodiment 45, wherein the support arm provides at least three degrees-of-freedom for the fluid injection device.

Embodiment 52: The surgical instrument of Embodiment 45, wherein at least one of the plurality of revolute joints is lockable in rotational orientation.

Embodiment 53: The surgical instrument of Embodiment 45, wherein the base is lockable in rotational orientation.

Embodiment 54: The surgical instrument of Embodiment 45, wherein the device adapter comprises a fluid injection device holder movably coupled to a rail.

Embodiment 55: The surgical instrument of Embodiment 54, wherein the fluid injection device holder comprises a clamp or clip for securing the fluid injection device.

Embodiment 56: The surgical instrument of Embodiment 54, wherein the fluid injection device holder comprises a tubular or ring-like body for securing the fluid injection device.

Embodiment 57: The surgical instrument of Embodiment 54, wherein the fluid injection device holder comprises a tubular or ring-like body for securing the fluid injection device.

Embodiment 58: The surgical instrument of Embodiment 54, wherein the fluid injection device holder is configured to linearly translate along the rail, thereby facilitating both rotational and translational movement for a fluid injection device when coupled to the device adapter.

Embodiment 59: A surgical instrument for fluid injection, the surgical instrument comprising: a handpiece configured for grasping by a user, the handpiece comprising a first lumen disposed therein; at least one actuatable toggle movably coupled with the handle; an articulable cannula coupled to the handpiece and configured to be introduced into an eye, the articulable cannula comprising a second lumen extending therethrough, the articulable cannula configured to articulate upon actuation of the at least one actuable toggle; and a needle movably disposed within the second lumen and coupled with the at least one actuatable toggle, the needle configured to extend from and retract into the second lumen at a distal end of the cannula upon actuation of the at least one actuable toggle.

Embodiment 60: The surgical instrument of Embodiment 59, wherein the articulable cannula comprises one or more features etched or cut into an outer surface of the articulable cannula to facilitate articulation thereof.

Embodiment 61: The surgical instrument of Embodiment 60, wherein the articulable cannula is formed of at least one of aluminum, stainless steel, polyether ether ketone (PEEK), polyetherketone (PEK), or polytetrafluoroethylene (PTFE).

Embodiment 62: The surgical instrument of Embodiment 59, further comprising: one or more wires coupled at one end to the at least one actuatable toggle and coupled at another end to one or more points along a length of the articulable cannula in the second lumen, wherein actuation of the at least one actuatable toggle causes the wires to act on the articulable cannula and manipulate a curvature of the articulable cannula.

Embodiment 63: The surgical instrument of Embodiment 59, the at least one actuatable toggle comprises a first toggle for extending and retracting the needle from the second lumen, and a second toggle for manipulating the articulable cannula.

Embodiment 64: A surgical instrument for fluid injection, the surgical instrument comprising: a handpiece configured for grasping by a user, the handpiece comprising a first lumen disposed therein; at least one actuatable toggle movably coupled with the handle; a cannula coupled to the handpiece and configured to be introduced into an eye, the cannula comprising a second lumen extending therethrough; a stiffening sleeve disposed around the cannula and configured to translate along a length of the cannula upon actuation of the at least one actuatable toggle, wherein distal translation of the stiffening sleeve increase a stiffness of the cannula and proximal translation of the stiffening sleeve decreases a stiffness of the cannula; and a needle movably disposed within the second lumen and coupled with the at least one actuatable toggle, the needle configured to extend from and retract into the second lumen at a distal end of the cannula upon actuation of the at least one actuable toggle.

Embodiment 65: The surgical instrument of Embodiment 64, wherein the stiffening sleeve comprises a hollow tubular body.

Embodiment 66: The surgical instrument of Embodiment 65, wherein the stiffening sleeve is formed of a metallic material comprising at least one of stainless steel, aluminum, or titanium.

Embodiment 67: The surgical instrument of Embodiment 65, wherein the stiffening sleeve is formed of a composite material comprising at least one of polyether ether ketone (PEEK), polyetherketone (PEK), polytetrafluoroethylene (PTFE), or polycarbonate (PC).

Embodiment 68: The surgical instrument of Embodiment 64, wherein a position of the stiffening sleeve along the length of the cannula is releasably lockable.

Embodiment 69: A surgical instrument for fluid injection, the surgical instrument comprising: a handpiece configured for grasping by a user, the handpiece comprising a first lumen disposed therein; at least one actuatable toggle movably coupled with the handle; a cannula coupled to the handpiece and configured to be introduced into an eye, the cannula comprising a second lumen extending therethrough; a stiffening sleeve disposed inside the cannula and configured to translate along a length of the cannula upon actuation of the at least one actuatable toggle, wherein distal translation of the stiffening sleeve increase a stiffness of the cannula and proximal translation of the stiffening sleeve decreases a stiffness of the cannula; and a needle movably disposed within the second lumen and coupled with the at least one actuatable toggle, the needle configured to extend from and retract into the second lumen at a distal end of the cannula upon actuation of the at least one actuable toggle.

Embodiment 70: The surgical instrument of Embodiment 69, wherein the stiffening sleeve comprises a hollow tubular body.

Embodiment 71: The surgical instrument of Embodiment 70, wherein the stiffening sleeve is formed of a metallic material comprising at least one of stainless steel, aluminum, or titanium.

Embodiment 72: The surgical instrument of Embodiment 70, wherein the stiffening sleeve is formed of a composite material comprising at least one of polyether ether ketone (PEEK), polyetherketone (PEK), polytetrafluoroethylene (PTFE), or polycarbonate (PC).

Embodiment 73: The surgical instrument of Embodiment 69, wherein a position of the stiffening sleeve along the length of the cannula is releasably lockable.

Embodiment 74: A surgical instrument for fluid injection, the surgical instrument comprising: a handpiece configured for grasping by a user, the handpiece comprising a first lumen disposed therein; at least one actuatable toggle movably coupled with the handle; a cannula indirectly coupled to the handpiece and configured to be introduced into an eye, the cannula comprising a second lumen extending therethrough; a needle movably disposed within the second lumen and coupled with the at least one actuatable toggle, the needle configured to extend from and retract into the second lumen at a distal end of the cannula upon actuation of the actuable toggle; and a shaft adapter coupling the cannula to the handpiece, the shaft adapter comprising a curvature such that a major longitudinal axis of at least a portion of the cannula is nonparallel with a manor longitudinal axis of the handpiece.

Embodiment 75: The surgical instrument of Embodiment 74, wherein the shaft adapter comprises a curved hollow tubular body.

Embodiment 76: The surgical instrument of Embodiment 74, wherein the cannula configured to extend from and retract into a distal end of the shaft adapter upon actuation of the at least one actuable toggle.

Embodiment 77: The surgical instrument of Embodiment 74, wherein the shaft adapter is formed of a metallic material comprising at least one of stainless steel, aluminum, or titanium.

Embodiment 78: The surgical instrument of Embodiment 74, wherein the shaft adapter has a radius of curvature between about 1 mm and about 20 mm.

Embodiment 79: An entry cannula for inserting a surgical instrument into an eye, the entry cannula comprising: a hollow body comprising a distal end, a proximal end, and a central channel extending from the distal end to the proximal end, wherein the hollow body comprises a non-circular cross-sectional profile; a distal portion disposed at the distal end of the hollow body, the distal portion comprising a wedge-like morphology; and a proximal portion disposed at the proximal end of the hollow tubular body, the proximal portion comprising a tube-like morphology.

Embodiment 80: The entry cannula of Embodiment 79, wherein the hollow body comprises a flattened cross-sectional profile having an elliptical, oval, or pill-like shape.

Embodiment 81: The entry cannula of Embodiment 79, wherein the distal portion comprises a cutout formed in a sidewall of the hollow body.

Embodiment 82: The entry cannula of Embodiment 79, wherein the distal portion and the proximal portion are formed of the same material.

Embodiment 83: The entry cannula of Embodiment 79, wherein the distal portion and the proximal portion are formed of different materials.

Embodiment 84: The entry cannula of Embodiment 79, further comprising one or more fixation arms coupled to and laterally extending from the proximal portion, the one of more fixation arms for immobilize the entry cannula upon insertion into the eye.

Embodiment 85: The entry cannula of Embodiment 84, wherein the one or more fixation arms are rigidly coupled to the proximal portion.

Embodiment 86: The entry cannula of Embodiment 84, wherein the one or more fixation arms are extendably coupled to the proximal portion such that the one or more fixation arms may be extended laterally outward from the proximal portion and retracted laterally inward toward the proximal portion.

Embodiment 87: An entry cannula for inserting a surgical instrument into an eye, the entry cannula comprising: a tube portion comprising a distal end and a proximal end, and a central channel extending from the distal end to the proximal end, the tube portion further comprising an endface disposed at the distal end and oriented at an non-normal angle relative to a major longitudinal axis of the tube portion to form a wedge-like morphology; and a funnel portion coupled to the proximal end of the tube portion, the funnel portion having a funnel-like morphology for facilitating insertion of the surgical instrument into the tube portion.

Embodiment 88: The entry cannula of Embodiment 87, wherein the funnel portion comprises a semi-funnel shape formed by a hyperbolic wall coupled to a planar wall.

Embodiment 89: The entry cannula of Embodiment 88, wherein a position of the planar wall of the funnel portion corresponds with an orientation of the endface of the tube portion.

Embodiment 90: A surgical instrument for fluid injection, the surgical instrument comprising: a handpiece configured for grasping by a user, the handpiece formed of a lightweight thermoplastic material, the handpiece comprising a first lumen disposed therein; an actuatable toggle movably coupled with the handle; a cannula coupled to the handpiece and configured to be introduced into an eye, the cannula comprising a second lumen extending therethrough; and a needle movably disposed within the second lumen and coupled with the actuatable toggle, the needle configured to extend from and retract into the second lumen at a distal end of the cannula upon actuation of the actuable toggle, wherein the surgical instrument is configured to hang freely upon insertion into the eye without damaging the eye.

Embodiment 91: The surgical instrument of Embodiment 90, wherein the lightweight thermoplastic material comprises at least one of polyether ether ketone (PEEK), polyetherketone (PEK), or polytetrafluoroethylene (PTFE).

Embodiment 92: The surgical instrument of Embodiment 90, wherein the actuable toggle comprises a sliding button.

Embodiment 93: The surgical instrument of Embodiment 90, wherein the handpiece comprises a fastening device disposed on an outer surface thereof, the fastening device for securing the surgical instrument to a patient.

Embodiment 94: The surgical instrument of Embodiment 93, wherein the fastening device comprises a velcro strip.

Embodiment 95: A system for performing an injection into a subretinal space of an eye, the system comprising: an expandable guidance cannula for traversing a suprachoroidal space of the eye, the expandable guidance cannula comprising: a flexible tubular member configured to expand laterally; and a first channel extending from a proximal end to a distal end of the flexible tubular member, wherein lateral expansion of the flexible tubular member increases a lateral dimension of the first channel for facilitating ingress of an injection cannula through the expandable guidance cannula; the injection cannula configured to be disposed through the first channel, the injection cannula comprising a lumen extending at least partially therethrough; and a needle movably disposed within the lumen, the needle configured to be extended from and retracted into the lumen at a distal end of the injection cannula.

Embodiment 96: The system of Embodiment 95, wherein the flexible tubular member further comprises a second channel disposed within a sidewall thereof, and wherein the flexible tubular member is expanded by filling the second channel with a working fluid.

Embodiment 97: The system of Embodiment 95, wherein the expandable guidance cannula further comprises a hub configured to contact a surface of the eye and anchor the expandable guidance cannula.

Embodiment 98: The system of Embodiment 97, wherein the hub comprises a port for filling a portion of the flexible tubular member with a working fluid to expand the flexible tubular member.

Embodiment 99: The system of Embodiment 97, wherein the hub a sloped inner surface for mechanically guiding the injection cannula during insertion of the injection cannula into the expandable guidance cannula.

Embodiment 100: The system of Embodiment 95, wherein the flexible tubular body is formed of at least one of silicone, polyurethane (PUR), polyether block amide (PEBA), or polyolefin.

Embodiment 101: The system of Embodiment 100, wherein the hub is formed of the same material as the flexible tubular body.

Embodiment 102: The system of Embodiment 100, wherein the hub is formed of a stiff or non-expandable material.

Embodiment 103: The system of Embodiment 95, wherein the flexible tubular member is coupled to a braided wire, and wherein the flexible tubular member is expanded by twisting the braided wire.

Embodiment 104: A surgical system for fluid injection, the surgical system comprising: an injection device, comprising: a handpiece configured for grasping by a user, the handpiece comprising a first lumen disposed therein; an actuatable toggle movably coupled with the handle; a cannula coupled to the handpiece and configured to be introduced into an eye, the cannula comprising a second lumen extending therethrough and a distal tip at a distal end of the cannula; and a needle movably disposed within the second lumen and coupled with the actuatable toggle, the needle configured to extend from and retract into the second lumen at the distal tip of the cannula upon actuation of the actuable toggle, wherein at least one of the distal tip or the needle is formed of a magnetic material; and one or more electromagnetic coils, the one or more electromagnetic coils configured to create a one-dimensional, two-dimensional, or three-dimensional magnetic field for guiding the injection device during traversal through the eye.

Embodiment 105: The system of Embodiment 104, wherein both the distal tip and the needle are formed of a magnetic material.

Embodiment 106: The system of Embodiment 104, wherein at least one of the one or more electromagnetic coils is integrated into a surgical head support, operating table, or surgical bed of an operating environment.

Embodiment 107: The system of Embodiment 104, wherein the one or more electromagnetic coils comprise three electromagnetic coils, and wherein one of the three electromagnetic coils is configured for placement above a patient's head, one of the three electromagnetic coils is configured for placement behind the patient's head, and another one of the three electromagnetic coils is configured for placement on either lateral side of the patient's head.

Embodiment 108: A system for performing an injection into a subretinal space of an eye, comprising: a memory comprising executable instructions; and a processor in data communication with the memory and configured to execute the instructions to cause the system to: receive a user input associated with an injection procedure; map the user input to one or more parameters for operating a fluidic drive system; and configure a surgical console to drive the fluidic drive system based on the one or more parameters for performing the injection procedure.

Embodiment 109: The system of Embodiment 108, wherein the one or more parameters comprise user-programmed parameters for operating the fluidic drive system.

Embodiment 110: The system of Embodiment 108, wherein the one or more parameters comprise universal parameters for operating the fluidic drive system.

Embodiment 111: The system of Embodiment 108, wherein the one or more parameters comprise parameters for controlling a fluidic flow of injection fluids.

Embodiment 112: The system of Embodiment 111, wherein the one or more parameters comprise at least one of fluid flow rate, fluid pressure, fluid delivery volume, or fluid delivery time.

Embodiment 113: The system of Embodiment 108, wherein the one or more parameters comprise parameters associated with a type of subretinal injection procedure.

Embodiment 114: The system of Embodiment 108, wherein the one or more parameters comprise subretinal injection sequence of operations.

Embodiment 115: The system of Embodiment 108, wherein the driving of the fluidic drive system based on the one or more parameters may be modified by additional user input.

Embodiment 116: The system of Embodiment 108, wherein the system is further configured to provide visual feedback to a user relating to the one or more parameters or a progress of the injection procedure.

Embodiment 117: The system of Embodiment 116, wherein the one or more parameters or the progress of the injection procedure is displayed on a display screen during performance of the injection procedure.

Embodiment 118: The system of Embodiment 108, wherein the system is further configured to provide auditory feedback to a user relating to the one or more parameters or a progress of the injection procedure.

Embodiment 119: The system of Embodiment 108, wherein driving the fluidic drive system based on the one or more parameters for performing the injection procedure is based on a continuous user input.

Embodiment 120: The system of Embodiment 119, wherein in the absence of the continuous user input, the controller is configured to cause the surgical console to cease performance of the injection procedure.

Embodiment 121: A surgical instrument for fluid injection, the surgical instrument comprising: a handpiece configured for grasping by a user, the handpiece comprising a first lumen disposed therein, the first lumen configured to receive a fluid cartridge comprising one or more injection fluids; a fluidic drive system disposed within the first lumen, the fluidic drive system for driving a flow of the one or more injection fluids from the fluid cartridge into a cannula; the cannula coupled to the handpiece and configured to be introduced into an eye, the cannula comprising a second lumen extending therethrough for receiving the one or more injection fluids flowed from the fluid cartridge; and a needle movably disposed within the second lumen, the needle configured to extend from and retract into the second lumen at a distal end of the cannula.

Embodiment 122: The system of Embodiment 121, wherein the fluidic drive system comprises an electromechanical actuator coupled to a piston, the electromechanical actuator configured to translate the piston within the first lumen, the piston configured to engage with the fluid cartridge for driving the flow of the one or more injection fluids therefrom.

Embodiment 123: The system of Embodiment 122, wherein the electromechanical actuator comprises an electromechanical linear or rotary stepper motor.

Embodiment 124: The system of Embodiment 123, wherein the electromechanical actuator comprises a rotary screw motor and the piston comprises a rotary lead screw, wherein rotation of the rotary lead screw by the rotary screw motor causes the rotary lead screw to translate linearly within the first lumen.

Embodiment 125: The system of Embodiment 121, wherein the fluidic drive system comprises an electro-pneumatic actuator coupled to a piston, the electro-pneumatic actuator configured to translate the piston within the first lumen, the piston configured to engage with the fluid cartridge for driving the flow of the one or more injection fluids therefrom.

Embodiment 126: The system of Embodiment 125, wherein the electro-pneumatic actuator comprises a pressurized fluid canister coupled to a valve, wherein adjusting a position of the valve modifies a flow rate of pressurized fluids from the pressurized fluid canister into the first lumen, the pressurized fluids in the first lumen acting upon the piston to translate the piston linearly within the first lumen.

Embodiment 127: The system of Embodiment 126, wherein the valve comprises at least one of a solenoid-type valve, a proportional valve, a plug valve, a piston valve, or a knife valve.

Embodiment 128: The system of Embodiment 121, wherein the fluidic drive system comprises spring-actuated mechanism configured to engage with the fluid cartridge for driving the flow of the one or more injection fluids therefrom.

Embodiment 129: The system of Embodiment 128, wherein the spring-actuated mechanism comprises a spring coupled to a piston, the spring providing a biasing force against the piston to translate the piston linearly within the first lumen.

Embodiment 130: The system of Embodiment 129, wherein the translation of the piston within the first lumen is further controlled by a brake coupled to the piston.

Embodiment 131: The system of Embodiment 121, wherein the fluid cartridge comprises a premixed treatment and non-treatment solution for injection.

Embodiment 132: The system of Embodiment 121, wherein the fluid cartridge comprises an unmixed treatment solution and non-treatment solution for injection, and wherein driving the flow of the one or more injection fluids from the fluid cartridge into a cannula comprises mixing the treatment solution and non-treatment solution.

Embodiment 133: A method of performing an injection into a subretinal space of an eye, the method comprising: inserting a distal end of a cannula into an intraocular space of the eye; guiding the distal end of the cannula to a target site on a surface of a retina of the eye; extending a needle from the cannula and through the retina into the subretinal space of the eye; injecting fluids from the injection cannula and the needle into the subretinal space; retracting the needle into the cannula; removing the cannula from the intraocular space; and sealing the target site on the surface of the retina.

Embodiment 134: The method of Embodiment 133, wherein the sealing comprises applying a graft over the target site.

Embodiment 135: The method of Embodiment 134, wherein the graft comprises a biological graft.

Embodiment 136: The method of Embodiment 135, wherein the graft comprises a cellular graft.

Embodiment 137: The method of Embodiment 135, wherein the graft comprises a human amniotic membrane (hAM) graft.

Embodiment 138: The method of Embodiment 134, wherein the graft comprises a polymer-based scaffold.

Embodiment 139: The method of Embodiment 138, wherein the graft comprises a polymeric nanofiber scaffold.

Embodiment 140: The method of Embodiment 133, wherein the sealing comprises applying a sealing solution over the target site.

Embodiment 141: The method of Embodiment 140, wherein the sealing solution comprises one or more human proteins or cellular attachment factors in solution.

Embodiment 142: The method of Embodiment 141, wherein the one or more human proteins or cellular attachment factors comprises at least one of fibrin, collagen, thrombin, fibronectin, or laminin.

Embodiment 143: The method of Embodiment 140, wherein the sealing solution comprises a polymeric hydrogel.

Embodiment 144: The method of Embodiment 143, wherein the sealing solution comprises a biopolymer comprising at least one of chitosan, hyaluronic acid, gelatin, alginate, methylcellulose, or collagen.

Embodiment 145: The method of Embodiment 133, wherein the sealing comprises treating the target site with a laser beam to cause photocoagulation at the target site.

Embodiment 146: The method of Embodiment 145, wherein the laser beam has a wavelength between about 400 and about 850 nm.

Embodiment 147: A surgical instrument for fluid injection, the surgical instrument comprising: a handpiece configured for grasping by a user, the handpiece comprising a first lumen disposed therein; an actuatable toggle movably coupled with the handle; a slider disposed within the first lumen and coupled to the actuable toggle; a cannula coupled to the handpiece and configured to be introduced into an eye, the cannula comprising a second lumen extending therethrough; an inner fluidic shaft movably disposed within the second lumen and extending along a length of the cannula, the inner fluidic shaft coupled to the slider at a proximal end of the inner fluidic shaft; a needle coupled to the inner fluidic shaft at a distal end of the inner fluidic shaft, the needle and inner fluidic shaft configured to extend from and retract into the second lumen at a distal end of the cannula upon actuation of the actuable toggle; and an optical fiber coupled to the inner fluidic shaft, wherein the optical fiber distally terminates at or near the distal end of the inner fluidic shaft, the optical fiber for imaging anatomical structures when the surgical instrument is disposed in the eye.

Embodiment 148: The surgical instrument of Embodiment 147, wherein the optical fiber is optically coupled to an OCT system to collect one-dimensional, two-dimensional, and/or three-dimensional images of the anatomical structures.

Embodiment 149: The surgical instrument of Embodiment 148, wherein the OCT system is configured to determine measurements of individual or collective physical parameters of the eye from images collected and transmitted by the optical fiber.

Embodiment 150: The surgical instrument of Embodiment 148, wherein the OCT system is configured to determine a position of the injection needle or inner fluidic shaft relative to the anatomical structures from images collected and transmitted by the optical fiber.

Embodiment 151: The surgical instrument of Embodiment 147, wherein the optical fiber is disposed through a bore formed in a wall of the inner fluidic shaft.

Embodiment 152: The surgical instrument of Embodiment 147, wherein the optical fiber is fixedly disposed in a groove formed in a wall of the inner fluidic shaft.

What is claimed is:

1. An apparatus for performing a subretinal injection into a subretinal space of an eye, the apparatus comprising:
an injection needle having a proximal end and a distal end, the distal end configured to be insertable into the subretinal space at a position on a surface of a retina;
an inserter device removably coupled to the injection needle;
a tubing having a distal end coupled to the proximal end of the injection needle and a proximal end coupled to a fluid source, the tubing having a first lumen and a second lumen, wherein the tubing is disposed through the inserter device;
a stabilizer configured to immobilize the injection needle at the position on the surface of the retina; and
the fluid source having a first fluid reservoir containing a non-treatment solution and a second fluid reservoir containing a treatment solution, wherein the fluid source is configured to provide the non-treatment solution from the first fluid reservoir to the subretinal space via the first lumen, and wherein the fluid source is configured to provide the treatment solution from the second fluid reservoir into the subretinal space via the second lumen.

2. The apparatus of claim 1, wherein the injection needle further comprises a connector piece, and wherein the stabilizer is movably coupled to the connector piece.

3. The apparatus of claim 2, wherein the stabilizer is configured to translate along the connector piece between an inactive position and an active position to immobilize the injection needle at the position on the surface of the retina, and wherein the stabilizer is disposed external to the connector piece in both the inactive position and the active position.

4. The apparatus of claim 3, wherein the stabilizer comprises a plurality of bendable legs, wherein the plurality of bendable legs are configured to be extended in the inactive position and bent in the active position.

5. The apparatus of claim 4, wherein the plurality of bendable legs are coupled to an extension ring at a proximal end of each of the plurality of bendable legs, wherein the extension ring circumscribes the connector piece and is configured to translate along the connector piece, and wherein distal movement of the extension ring causes the plurality of bendable legs to bend.

6. The apparatus of claim 1, wherein the tubing further comprises a third lumen, wherein the stabilizer is movably coupled to a distal end of the third lumen, and wherein pressure or fluid applied through the third lumen is configured to extend the stabilizer beyond the distal end of the third lumen.

7. The apparatus of claim 6, wherein the fluid source further comprises a third fluid reservoir, and wherein the fluid source is configured to provide a working fluid from the third fluid reservoir via the third lumen to extend the stabilizer.

8. The apparatus of claim 1, wherein the stabilizer comprises an elastic balloon.

9. The apparatus of claim 8, wherein the balloon is extended beyond the distal end of a third lumen of the tubing by filling with a working fluid comprising at least one of perfluorocarbon liquid (PFCL), BSS, saline, air, or N2.

10. The apparatus of claim 1, wherein the inserter device is configured to be decoupled from the tubing outside the eye after being decoupled from the injection needle.

11. A method of performing a subretinal injection into a subretinal space of an eye, the method comprising:
inserting a distal end of an injection needle into the subretinal space at a target site on a surface of a retina, the injection needle having a proximal end coupled to a distal end of a tubing, the tubing having a proximal end coupled to a fluid source, the proximal end of the injection needle further removably coupled to a distal end of an inserter device;
immobilizing the injection needle at the target site on the surface of the retina by applying a pressure or fluid through a first lumen of the tubing to extend a stabilizer beyond a distal end of the first lumen to contact the surface of the retina;
decoupling the inserter device from the injection needle;
providing a non-treatment solution from the fluid source to the subretinal space via a second lumen of the tubing; and
providing a treatment solution to the subretinal space via a third lumen of the tubing using the fluid source.

12. The method of claim 11, wherein providing each of the non-treatment solution and the treatment solution is performed hands-free.

13. The method of claim 11, wherein providing each of the non-treatment solution and the treatment solution is performed simultaneously.

14. The method of claim 11, wherein providing each of the non-treatment solution and the treatment solution is performed sequentially.

15. The method of claim 11, wherein the stabilizer is coupled to the distal end of the first lumen, and wherein applying the pressure or fluid through the first lumen comprises providing a working fluid from the fluid source to the first lumen.
